(19) United States
(12) Patent Application Publication
(10) Pub. No.: US 2005/0025824 A1
(43) Pub. Date: Feb. 3, 2005

(54) Pulsatile release histamine H2 antagonist dosage form

(75) Inventors: Phillip J. Percel, Troy, OH (US); Nehal H. Vyas, Vandalia, OH (US); Krishna S. Vishnupad, Dayton, OH (US); Gopi M. Venkatesh, Dayton, OH (US)

Correspondence Address: ARENT FOX KINTNER PLOTKIN & KAHN, 1050 CONNECTICUT AVENUE, N.W., SUITE 400, WASHINGTON, DC, 20036, US

(73) Assignee: EURAND PHARMACEUTICALS LTD.

(21) Appl. No.: 10/875,627

(22) Filed: Jun. 25, 2004

Related U.S. Application Data
(63) Continuation-in-part of application No. 10/689,566, filed on Oct. 20, 2003.
Application No. 10/689,566 is a continuation of application No. 10/057,759, filed on Jan. 25, 2002, now Pat. No. 6,663,888.
(60) Provisional application No. 60/340,419, filed on Dec. 14, 2001.

(51) Int. Cl. A61K 9/52; A61K 9/20

(52) U.S. Cl. 424/464; 514/400

(57) ABSTRACT

A unit dosage form, such as a capsule or the like, for delivering drugs into the body in a circadian release fashion comprising one or more populations of drug-containing particles (beads, pellets, granules, etc.) is disclosed. Each bead population exhibits a pre-designed rapid or sustained release profile with or without a predetermined lag time of 3 to 5 hours. Such a circadian rhythm release drug delivery system is designed to provide a plasma concentration-time profile, which varies according to physiological need at different times during the dosing period, i.e., mimicking the circadian rhythm and severity/manifestation of gastric acid secretion (and/or midnight gerd), predicted based on pharmaco-kinetic and pharmaco-dynamic considerations and in vitro/in vivo correlations.

### CROSS REFERENCES

This application claims the benefit of U.S. patent application Ser. No. 10/689,566 filed Oct. 20, 2003, which is a continuation of U.S. patent application Ser. No. 10/057,759 filed Jan. 25, 2002, which is a non-provisional application of U.S. Provisional Application No. 60/340,419 filed Dec. 14, 2001. The disclosure of the prior applications are hereby incorporated by reference herein in their entirety.

### TECHNICAL FIELD

A major objective of chronotherapy for indications such as asthma, gastric acid secretion, gastro-intestinal disorders, such as acid peptic disease, and cardiovascular diseases is to deliver the drug in higher concentrations during the time of greatest need and in lesser concentrations when the need is less. Types of acid peptic disease include “GERD” (Gastroesophageal Reflux Disease), heartburn, erosions and ulcerations (ulcers), Nocturnal Acid Breakthrough, nighttime heartburn, regurgitation, or retrosternal pain. Symptoms associated with GERD vary in severity throughout a 24-hour period. Delayed gastric emptying (abnormal gastric motility), involves backwashing of acid and bile into the esophagus and may also be associated with and/or contribute to GERD.

Accordingly, higher plasma concentrations of a histamine H₂ antagonist, such as nizatidine, are required to provide relief from acid secretion in response to fatty meals, as well as to attenuate the “midnight gerd” seen to occur in patients in response to the circadian rhythm to gastric acid secretion, while lower plasma concentrations are adequate in early morning hours and between meals. This is accomplished by administering a pulsatile release dosage form of the present invention, which provides a controlled release of an histamine H₂ antagonist from properly designed dosage forms. In particular, the present invention relates to a unit dosage form of an assembly of two or more bead populations, each of which is designed to release the therapeutic agent as a rapid or sustained release pulse after a predetermined delay with resulting plasma concentration varying in a circadian rhythm fashion, thereby enhancing patient compliance and therapeutic efficacy, reducing both cost of treatment and side effects.

### BACKGROUND OF THE INVENTION

Many therapeutic agents are most effective when made available at a constant rate at or near the absorption site. The absorption of therapeutic agents thus made available generally result in desired plasma concentrations leading to maximum efficacy, minimum toxic side effects. Much effort has been devoted to developing sophisticated drug delivery systems, such as osmotic devices, for oral application. However, there are instances where maintaining a constant blood level of a drug is not desirable. For example, a “position-controlled” drug delivery system (e.g., treatment of colon disease or use of colon as an absorption site for peptide and protein based products) may prove to be more efficacious. A pulsatile delivery system is capable of providing one or more immediate release pulses at predetermined time points after a controlled lag time or at specific sites. However, there are only a few such orally applicable pulsatile release systems due to the potential limitation of the size or materials used for dosage forms. Ishino et al. disclose a dry-coated tablet form in Chemical Pharm. Bull. Vol. 40 (11), 3036-041 (1992). U.S. Pat. No. 4,851,229 to Magruder et al., U.S. Pat. No. 5,011,692 to Fujioka et al., U.S. Pat. No. 5,017,381 to Maruyama et al., U.S. Pat. No. 5,229,135 to Philippon et al., and U.S. Pat. No. 5,840,329 to Bai disclose preparation of pulsatile release systems. Some other devices are disclosed in U.S. Pat. No. 4,871,549 to Ueda et al. and U. S. Pat. Nos. 5,260,068; 5,260,069; and 5,508,040 to Chen. U. S. Pat. Nos. 5,229,135 and 5,567,441 both to Chen disclose a pulsatile release system consisting of pellets coated with delayed release or water insoluble polymeric membranes incorporating hydrophobic water insoluble agents or enteric polymers to alter membrane permeability. U.S. Pat. No. 5,837,284 to Mehta et al. discloses a dosage form which provides an immediate release dose of methylphenidate upon oral administration, followed by one or more additional doses spread over several hours.

The relationship between plasma nizatidine concentrations and inhibition of basal and protein-stimulated gastric acid secretions previously was investigated in 5 healthy subjects. Schneck et al. Clin. Pharmacol. Ther. 47: 499-503 (1990). The results of this study showed basal acid secretion and protein-stimulated acid secretion were inhibited by 90% at mean plasma nizatidine concentrations of 430 and 490 ng/mL, respectively.

Studies have shown that gastric acid secretion, especially the midnight gerd, follows a circadian rhythm. In such cases, administration of a different kind of unit dosage form which delivers the drug in higher concentrations during the time of greatest need, for example, around dinner and close to midnight, and in lesser concentrations at other times, is needed. Commonly assigned and co-pending U.S. application Ser. No. 09/778,645, which is incorporated in its entirety, discloses a pulsatile release system comprising a combination of two or three pellet populations, each with a well-defined release profile. In accordance with the present invention, a plasma profile is obtained which varies in a circadian rhythm fashion following administration of the novel dosage form.

### SUMMARY OF THE INVENTION

In embodiments, this invention is directed to a histamine H₂ antagonist pharmaceutical dosage form providing a bi-modal pulsatile release profile comprising immediate release (IR) beads comprising an active-containing core particle and timed pulsatile release (TPR) beads, wherein said TPR beads comprise an active-containing core particle and a pulse coating surrounding said core, wherein said IR beads provide a therapeutically effective amount of active to treat gastric acid secretions and the TPR beads provide a delayed dose of active which provides a therapeutically effective amount of active to treat midnight GERD.

In embodiments, this invention is directed to a method for the preparation of the above dosage form, comprising the steps of preparing a nizatidine-containing core to form IR beads, coating a fraction of the IR beads with a mixture of plasticized ethylcellulose and an enteric polymer to form TPR beads, and filling capsules with IR beads and TPR beads at a ratio from about 3:1 to about 1:3.

In embodiments, this invention is directed to a pulsatile release nizatidine dosage form comprising immediate release (IR) beads comprising a nizatidine-containing core particle and timed pulsatile release (TPR) beads, wherein said TPR beads comprise a nizatidine-containing core particle and a pulse coating surrounding said core, said pulse coating comprising ethylcellulose and an enteric polymer; wherein said TPR beads when tested in a USP type II apparatus at 50 rpm using a 2-stage dissolution medium (first 2 hours and 700 ml 0.1 N HCl at 37° C. followed by a dissolution in a pH of 6.8 obtained by the addition of 200 ml of pH modifier) exhibits a dissolution profile substantially corresponding to the following pattern: after 2 hours, about 0-25% of the total nizatidine is released; after 3 hours, about 15-80% of the total nizatidine is released; and after 4 hours, not less than 60% of the total nizatidine is release.

In embodiments, this invention is directed to a method of treating a human having a gastro-intestinal disorder, comprising administering to the human once daily a bi-modal pulsatile release oral pharmaceutical dosage form comprising immediate release (IR) beads comprising a core particle containing nizatidine, timed pulsatile release (TPR) beads, wherein said TPR beads comprise a core particle containing nizatidine, and a pulse coating surrounding said core.

In embodiments, this invention is directed to a method of administering nizatidine, comprising administering orally to a human a bi-modal pulsatile release formulation comprising nizatidine that provides two peak blood plasma concentrations of nizatidine occurring from about 2.0 to about 4.0 hours apart, wherein the first peak concentration occurs within 2 hours after administration and wherein a therapeutic level of nizatidine is maintained for about 6 to about 8 hours after administration.

In embodiments, this invention is directed to a method of administering nizatidine, comprising administering orally to a human a bi-modal pulsatile release formulation producing a first peak blood plasma concentration and a second peak blood level concentration, wherein the ratio of the first peak to the second peak is between about 75:25 and about 25:75, preferably between about 67:33 and about 33:67. The formulation may comprise immediate release (IR) beads comprising a core particle containing nizatidine and timed pulsatile release (TPR) beads, wherein said TPR beads comprise a core particle containing nizatidine and a pulse coating surrounding said core.

In embodiments, this invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 7.

In embodiments, this invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 8.

In embodiments, this invention is directed to an extended release (ER) oral dosage form comprising 150 mg of nizatidine, which after oral administration of a single one of said dosage forms twice daily in an adult human produces a blood plasma concentration of nizatidine ranging from 80 to 120% of the blood plasma concentration values per time period over the first twenty four hours shown in FIG. 9.

In embodiments, this invention is directed to an extended release (ER) oral dosage form comprising 150 mg of nizatidine, which after oral administration of two of said dosage forms once daily in an adult human produces a blood plasma concentration of nizatidine ranging from 80 to 120% of the blood plasma concentration values per time period over the first eight hours shown in FIG. 9.

In embodiments, this invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in any one of FIGS. 10-55.

In embodiments, this invention is directed to an extended release (ER) oral dosage form comprising 150 mg of nizatidine, which after oral administration of a single one of said dosage forms in an adult human produces a blood plasma concentration of n-desmethylnizatidine ranging from 80 to 120% of the blood plasma concentration values per time period over the first eight hours shown in any one of FIGS. 56-101.

### BRIEF DESCRIPTION OF THE DRAWINGS

The invention will be described in further detail with reference to the accompanying Figures wherein:

FIG. 1 shows Circadian Rhythm variations in gastric acid secretion (Reference: the presentation by Gordon L. Amidon at the Formulation Optimization and Clinical Pharmacology, a Capsugel Sponsored Conference at Tokyo, Apr. 23, 1999, p. 16).

FIG. 2 shows the drug release profiles from Nizatidine Pulsatile Capsules, 150 mg (75 mg IR Beads+75 mg TPR Beads) of Example 1, wherein the TPR Beads have different pulse coating levels.

FIG. 3 shows the drug release profile for Nizatidine Pulsatile Capsules, 150 mg (75 mg IR Beads+75 mg TPR Beads) of Example 2.

FIG. 4 shows the target or simulated in vitro drug release profile used in PK simulation.

FIG. 5 compares the simulated plasma levels of Nizatidine Pulsatile Capsule versus 300 mg IR Dose following oral administration (a) in the evening and (b) during the day time.

FIG. 6 shows the plasma level of Nizatidine following oral administration in a healthy volunteer when dosed after dinner with Pulsatile Capsule, 150 mg (75 mg IR Beads+75 mg TPR Beads) (a bimodal display) versus 150 mg IR Dose.

FIGS. 7A and 7B compare the plasma levels of Nizatidine in a fasted normal healthy male subjects of Nizatidine Pulsatile Capsule, 150 mg versus Axid® 150 mg following oral administration.

FIGS. 8A and 8B compare the plasma levels of Nizatidine following oral administration of Nizatidine Pulsatile Capsule, 150 mg in fed versus fasted normal healthy male subjects.

FIGS. 9A and 9B compare the plasma levels of Nizatidine following oral administration in a healthy subject when dosed with Nizatidine ER 150 mg bid versus nizatidine ER 300 mg qd versus Axid® 150 mg bid.

FIGS. 10-32A and B summarize the plasma levels of Nizatidine in representative normal healthy male subjects, following oral administration of Nizatidine Pulsatile Capsule, 150 mg versus Axid® 150 mg.

FIGS. 33-55A and B summarize the plasma levels of Nizatidine in representative normal healthy male subjects, following oral administration of Nizatidine Pulsatile Capsule in fed versus fasted conditions.

FIGS. 56-78A and B summarize the plasma levels of n-Desmethylnizatidine in representative normal healthy male subjects, following oral administration of Nizatidine Pulsatile Capsule, 150 mg versus Axid® 150 mg.

FIGS. 79-101A and B summarize the plasma levels of n-Desmethylnizatidine in representative normal healthy male subjects, following oral administration of Nizatidine Pulsatile Capsule in fed versus fasted conditions.

FIGS. 102A and 102B compare the plasma levels of n-Desmethylnizatidine in a fasted normal healthy male subjects of Nizatidine Pulsatile Capsule, 150 mg versus Axid® 150 mg following oral administration.

FIGS. 103A and 103B compare the plasma levels of n-Desmethylnizatidine following oral administration of Nizatidine Pulsatile Capsule, 150 mg in fed versus fasted normal healthy male subjects.

FIGS. 104A and 104B compare the plasma levels of n-Desmethylnizatidine following oral administration in a healthy subject when dosed with Nizatidine ER 150 mg bid versus nizatidine ER 300 mg qd versus Axid® 150 mg bid.

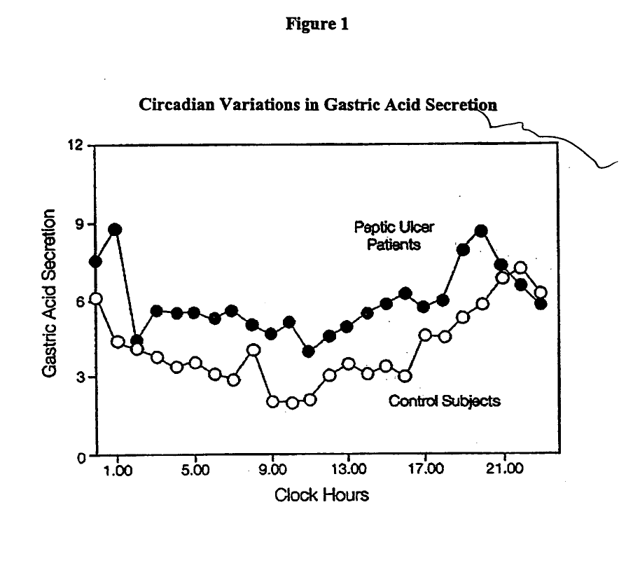

### DETAILED DESCRIPTION OF THE INVENTION

The present invention provides a pulsatile release, multi-particulate dosage form comprising a mixture of two types of beads comprising a histamine H₂ receptor antagonist: IR (Immediate Release) Beads and TPR (Timed Pulsatile Release) Beads. IR (immediate release) Beads allow immediate release of the active while TPR Beads allow a delayed “burst” release (timed pulsatile release) of the active after a lag of 3-4 hours. When administered at bedtime (capsule containing IR beads+TPR beads), the immediate release of the active is intended to provide relief from acid secretion in response to the meal, while the delayed “burst” is intended to attenuate the “midnight gerd” seen to occur in patients in response to the circadian rhythm to gastric acid secretion. Release profiles which approximate the daily fluctuations in gastric acid secretion are obtainable by blending IR Beads and TPR Beads at an appropriate ratio estimated from pharmaco-kinetic modeling.

The active core of the novel dosage form of the present invention may be comprised of an inert particle or an acidic or alkaline buffer crystal, which is coated with a drug-containing film-forming formulation and preferably a water-soluble film forming composition to form a water-soluble/dispersible particle. Alternatively, the active may be prepared by granulating and milling and/or by extrusion and spheronization of a polymer composition containing the drug substance. The amount of drug in the core will depend on the dose that is required, and typically varies from about 5 to 90 weight %.

The IR Beads typically comprise two coatings applied to non-pareil seeds (# 25-30 mesh). The first coating contains a histamine H₂ antagonist and a binder, such as hydroxypropyl cellulose. The drug layered beads are coated with a seal coating of Opadry Clear to produce IR Beads. TPR Beads can be produced by applying a second functional membrane comprising a mixture of water insoluble polymer and an enteric polymer to IR Beads, both plasticized polymeric systems being applied from aqueous or solvent based systems.

Generally, the polymeric coating on the active core will be from about 1 to 50% based on the weight of the coated particle, depending on the lag time and type of release profile required and/or the polymers and coating solvents chosen. Those skilled in the art will be able to select an appropriate amount of drug for coating onto or incorporating into the core to achieve the desired dosage. In one embodiment, the inactive core may be a sugar sphere or a buffer crystal or an encapsulated buffer crystal such as calcium carbonate, sodium bicarbonate, fumaric acid, tartaric acid, etc. which alters the microenvironment of the drug to facilitate its release.

To produce Timed Pulsatile Release (TPR) Beads, a water soluble/dispersible drug-containing particle is coated with a mixture of a water insoluble polymer and an enteric polymer, wherein the water insoluble polymer and the enteric polymer may be present at a weight ratio of from 4:1 to 1:1, and the total weight of the coatings is 10 to 60 weight % based on the total weight of the coated beads. The drug layered beads may optionally include an inner dissolution rate controlling membrane of ethylcellulose. The composition of the outer layer, as well as the individual weights of the inner and outer layers of the polymeric membrane are optimized for achieving desired circadian rhythm release profiles for a given active, which are predicted based on in vitro/in vivo correlations.

In accordance with one embodiment of the present invention, a unit dosage form is provided wherein the unit dose comprises a mixture of immediate release beads (IR Beads, which are drug-containing particles without a dissolution rate controlling polymer membrane) and TPR Beads (drug containing particles with a coating of a blend of water insoluble polymer and enteric polymer exhibiting a lag time of 2-4 hours following oral administration), thus providing a two-pulse release profile. The IR beads provide a loading dose by releasing substantially all of the active contained in said IR beads within the first three hours after administration of the dosage form, preferably the first two hours, even more preferably the first hour after administration of the dosage form. A unit dosage form, which does not comprise a rapid release bead population acting as a bolus dose, is also an embodiment of the present invention.

The present invention also provides a method of making a pulsatile release dosage form comprising a mixture of two bead populations comprising the steps of:
1. preparing a drug-containing core by coating an inert particle such as a non-pareil seed, an acidic buffer crystal or an alkaline buffer crystal with a drug and a polymeric binder or by granulation and milling or by extrusion/spheronization to form an immediate release (IR) bead;
2. coating the IR bead with a mixture of plasticized water-insoluble and enteric polymers to form a Timed Pulsatile Release (TPR) bead;
3. filling into hard gelatin capsules IR beads and TPR beads at a proper ratio to produce pulsatile capsules providing the desired release profile.

The release profile for TPR beads can be determined according to the following procedure:

Dissolution Procedure:

Dissolution Apparatus: USP Apparatus 2 (Paddles at 50 rpm) using a two-stage dissolution medium (first 2 hrs in 700 mL 0.1N HCl at 37° C. followed by dissolution at pH=6.8 obtained by the addition of 200 mL of pH modifier) and Drug Release determination by HPLC).

The TSR Beads prepared in accordance with present invention release, when tested by the above procedure, not more than 25%, more preferably not more than 15%, and most preferably not more than 5% in 2 hours, about 15-80%, more preferably about 20-65%, and most preferably about 30-50% in 3 hours, and not less than 60%, more preferably not less than 70%, and most preferably not less than 80% in 4 hrs.

Dosage forms in accordance with the present invention typically comprise a combination of IR Beads and TPR Beads at a ratio from 3:1 to 1:3, preferably a ratio from 2:1 to 1:2. In accordance with certain embodiments, the ratio of IR Beads to TPR Beads is approximately 1:1.

The histamine H₂ receptor antagonists suitable for incorporation into these circadian rhythm release (CRR) drug delivery systems include acidic, basic, zwitterion, or neutral bioactive molecules or their salts indicated for the treatment of active duodenal ulcer, such as nizatidine, cimetidine, ranitidine, famotidine and derivatives thereof.

An aqueous or a pharmaceutically acceptable solvent medium may be used for preparing drug-containing core particles. The type of film forming binder that is used to bind the drug to the inert sugar sphere is not critical but usually water soluble, alcohol soluble or acetone/water soluble binders are used. Binders such as polyvinylpyrrolidone (PVP), polyethylene oxide, hydroxypropyl methylcellulose (HPMC), hydroxypropylcellulose (HPC), polysaccharides such as dextran, corn starch may be used at concentrations of 0.5 to 5 weight %. The drug substance may be present in this coating formulation in the solution form or may be dispersed at a solid content up to 35 weight % depending on the viscosity of the coating formulation.

The drug substance, a binder such as PVP, a dissolution rate controlling polymer (if used), and optionally other pharmaceutically acceptable excipients are blended together in a planetary mixer or a high shear granulator such as Fielder and granulated by adding/spraying a granulating fluid such as water or alcohol. The wet mass can be extruded and spheronized to produce spherical particles (beads) using an extruder/marumerizer. In these embodiments, the drug load could be as high as 90% by weight based on the total weight of the extruded/spheronized core.

The active containing cores (beads, pellets or granular particles) thus obtained may be coated with one or two layers of dissolution rate controlling polymers to obtain desired release profiles with or without a lag time. The inner layer membrane largely controls the rate of drug release following imbibition of water or body fluids into the core while the outer layer membrane provides for the desired lag time (the period of no or little drug release following imbibition of water or body fluids into the core). The inner layer membrane may comprise a water insoluble polymer, or a mixture of water insoluble and water soluble polymers. Representative examples of water insoluble polymers useful in the invention include ethylcellulose, polyvinyl acetate (Kollicoat SR#0D from BASF), neutral copolymers based on ethyl acrylate and methylmethacrylate, copolymers of acrylic and methacrylic acid esters with quaternary ammonium groups such as Eudragit NE, RS and RS30D, RL or RL30D and the like, preferably ethylcellulose. Representative examples of water soluble polymers are low molecular weight HPMC, HPC, methylcellulose, polyethylene glycol (PEG of molecular weight>3000) at a thickness ranging from 1 weight % up to 10 weight % depending on the solubility of the active in water and the solvent or latex suspension based coating formulation used. The water insoluble polymer to water soluble polymer may typically vary from 95:5 to 60:40, preferably from 80:20 to 65:35.

The polymers suitable for the outer membrane, which largely controls the lag time of up to 6 hours may comprise an enteric polymer and a water insoluble polymer at a thickness of 10 to 50 weight %. The ratio of water insoluble polymer to enteric polymer may vary from 4:1 to 1:2, preferably the polymers are present at a ratio of about 2:1 to about 1:1. Even more preferably, the ratio is approximately 1:1 where the enteric polymer is hydroxypropyl methylcellulose phthalate. The water insoluble polymer typically used is ethylcellulose.

Representative examples of enteric polymers useful in the invention include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methacrylate copolymers, shellac and derivatives thereof. These polymers may be used as a dry powder or an aqueous dispersion. Some commercially available materials that may be used are methacrylic acid copolymers sold under the trademark Eudragit (L100, S100, L30D) manufactured by Rhom Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Both enteric and water insoluble polymers used in forming the membranes are usually plasticized. Representative examples of plasticizers that may be used to plasticize the membranes include triacetin, tributyl citrate, triethyl citrate, acetyl tri-n-butyl citrate diethyl phthalate, castor oil, dibutyl sebacate, acetylated monoglycerides and diglycerides or mixtures thereof. The plasticizer may comprise about 3 to 30 wt. % and more typically about 10 to 25 wt. % based on the polymer. The type of plasticizer and its content depends on the polymer or polymers, nature of the coating system (e.g., aqueous or solvent based, solution or dispersion based and the total solids).

In general, it is desirable to prime the surface of the particle before applying the pulsatile release membrane coatings or to separate the different membrane layers by applying a thin hydroxypropyl methylcellulose (HPMC) (Opadry Clear) film. While HPMC is typically used, other primers such as hydroxypropylcellulose (HPC) can also be used.

The membrane coatings can be applied to the core using any of the coating techniques commonly used in the pharmaceutical industry, but fluid bed coating is particularly useful.

The present invention is applied to multi-dose forms, i.e., drug products in the form of multi-particulate dosage forms (pellets, beads, granules or mini-tablets) or in other forms suitable for oral administration. Administration may be once or twice daily. Administration is preferably in the evening, i.e. from about 5p.m. to about 12 a.m, more preferably from about 6 p.m. to about 8 p.m., even more preferably at about 6 p.m., and/or in the morning, i.e. within six hours of waking, more preferably within 4 hours of waking.

In embodiments, the present invention is directed to a histamine H₂ antagonist pharmaceutical dosage form providing a bi-modal pulsatile release profile comprising immediate release (IR) beads comprising an active-containing core particle and timed pulsatile release (TPR) beads, wherein said TPR beads comprise an active-containing core particle and a pulse coating surrounding said core, wherein said IR beads provide a therapeutically effective amount of active to treat gastric acid secretions and the TPR beads provide a delayed dose of active which provides a therapeutically effective amount of active to treat midnight GERD. Histamine H₂ receptor antagonist suitable for the present invention include nizatidine, cimetidine, ranitidine, and famotidine and derivatives thereof. Preferably, the timed pulsatile release (TPR) beads when tested in a USP Type II apparatus at 50 rpm using a 2-stage dissolution medium (first 2 hours and 700 ml 0.1 N HCl at 37° C. followed by a dissolution in a pH of 6.8 obtained by the addition of 200 ml of pH modifier) exhibits a dissolution profile substantially corresponding to the following pattern: after 2 hours, about 0-25% of the total active is released; after 3 hours, about 15-80% of the total active is released; and after 4 hours, not less than 60% of the total active is released. Even more preferably the dissolution profile substantially corresponds to the following pattern: after 2 hours, about 0-15% of the total active is released; after 3 hours, about 20-65% of the total active is released; and after 4 hours, not less than 70% of the total active is released. Most preferably, the dissolution profile substantially corresponds to the following pattern: after 2 hours, about 0-5% of the total active is released; after 3 hours, about 30-50% of the total active is released; and after 4 hours, not less than 80% of the total active is released.

The pulse coating of the embodiments comprise a water insoluble polymer and an enteric polymer. The enteric polymer is selected from the group consisting of esters of cellulose, polyvinyl acetate phthalate, pH-sensitive methacrylic acid-methylmethacrylate copolymers, shellac and derivatives thereof. Preferably, the enteric polymer is selected from the group consisting of cellulose acetate phthalate, hydroxypropyl methylcellulose phthalate, hydroxypropyl methylcellulose succinate and combinations thereof.

Further, at least one of said polymers may further comprise a plasticizer. Plasticizers suitable for the present invention include triacetin, tributyl citrate, tri-ethyl citrate, acetyl tri-n-butyl citrate, diethyl phthalate, dibutyl sebacate, polyethylene glycol, polypropylene glycol, castor oil and acetylated mono- and di-glycerides and mixtures thereof.

Preferably, the water insoluble polymer and the enteric polymer are present in the pulse coating at a ratio from about 4:1 to about 1:2, more preferably from about 2:1 to about 1:2. Even more preferably, the water insoluble polymer is ethylcellulose and said enteric polymer is hydroxypropyl methylcellulose phthalate, such that the ratio is about 1:1.

Preferably the IR beads and TPR beads are present in a ratio from about 3:1 to about 1:3, more preferably from about 2:1 to about 1:2. Preferably, the IR beads release substantially all of the active contained therein within the first hour after administration of the dosage form. It also preferred that the total weight of the coating on the TPR beads is about 10-60 weight % based on the total weight of the TPR beads.

In embodiments, the present invention is directed to a method for the preparation of a dosage form, comprising the steps of preparing an active-containing core to form IR beads, coating a fraction of the IR beads with a water insoluble polymer and an enteric polymer to form TPR beads, and filling capsules with IR beads and TPR beads at a ratio from about 3:1 to about 1:3. The active-containing core is produced by coating a particle selected from the group consisting of non-pareil seeds, acidic buffer crystals and alkaline buffer crystals with a water soluble film-forming composition comprising nizatidine and a polymeric binder. Alternately, the active-containing core is produced by granulating and milling and/or by extruding and spheronizing a polymer composition containing nizatidine.

In embodiments, the invention is directed to a pulsatile release nizatidine dosage form comprising immediate release (IR) beads comprising a nizatidine-containing core particle; and timed pulsatile release (TPR) beads, wherein said TPR beads comprise: a nizatidine-containing core particle and a pulse coating surrounding said core, said pulse coating comprising ethylcellulose and an enteric polymer, wherein said TPR beads when tested in a USP type II apparatus at 50 rpm using a 2-stage dissolution medium (first 2 hours and 700 ml 0.1 N HCl at 37° C. followed by a dissolution in a pH of 6.8 obtained by the addition of 200 ml of pH modifier) exhibits a dissolution profile substantially corresponding to the following pattern: after 2 hours, about 0-25% of the total nizatidine is released; after 3 hours, about 15-80% of the total nizatidine is released; and after 4 hours, not less than 60% of the total nizatidine is released. Preferably, the dissolution profile substantially corresponds to the following pattern: after 2 hours, about 0-15% of the total nizatidine is released; after 3 hours, about 20-65% of the total nizatidine is released; and after 4 hours, not less than 70% of the total nizatidine is released. Even more preferable is when the dissolution profile substantially corresponds to the following pattern: after 2 hours, about 0-5% of the total nizatidine is released; after 3 hours, about 30-50% of the total nizatidine is released; and after 4 hours, not less than 80% of the total nizatidine is released.

The core particle is a non-pareil sugar seed coated with nizatidine and a polymeric binder, or the core particle is prepared by granulating and milling and/or by extruding and spheronizing a polymer composition containing nizatidine, to form a core particle containing nizatidine. The enteric polymer is selected from the group consisting of esters of cellulose, polyvinyl acetate phthalate, pH-sensitive methacrylic acid-methylmethacrylate copolymers, shellac and derivatives thereof. Preferably, the enteric polymer is selected from the group consisting of cellulose acetate phthalate, hydroxypropyl methylcellulose phthalate, hydroxypropyl methylcellulose succinate and combinations thereof.

The pulse coating may also comprise a plasticizer. Plasticizers suitable for the present invention include triacetin, tributyl citrate, tri-ethyl citrate, acetyl tri-n-butyl citrate, diethyl phthalate, dibutyl sebacate, polyethylene glycol, polypropylene glycol, castor oil and acetylated mono- and di-glycerides and mixtures thereof.

Where ethylcellulose and the enteric polymer are present in said pulse release coating at a ratio from about 4:1 to about 1:2, preferably from about 2:1 to about 1:2. Where the enteric polymer is hydroxypropyl methylcellulose phthalate, the ratio is about 1:1.

The IR beads and the TPR beads are preferably present in a ratio from about 3:1 to about 1:3, more preferably in a ratio from about 2:1 to about 1:2. The IR beads preferably contain a total of about 50-100 mg of nizatidine, more preferably 75 mg of nizatidine, and said TPR beads preferably contain a total of about 50-100 mg of nizatidine, more preferably 75 mg of nizatidine. The IR beads preferably release substantially all of the nizatidine contained therein within the first hour after administration of the dosage form. The total weight of the coating on the TPR beads is preferably about 10-60 weight % based on the total weight of the TPR beads.

In embodiments, the present invention is directed to a method for the preparation of the dosage form, comprising the steps of preparing a nizatidine-containing core to form IR beads, coating a fraction of the IR beads with a mixture of plasticized ethylcellulose and an enteric polymer to form TPR beads, and filling capsules with IR beads and TPR beads at a ratio from about 3:1 to about 1:3. The nizatidine-containing core is produced by coating a particle selected from the group consisting of non-pareil seeds, acidic buffer crystals and alkaline buffer crystals with a water soluble film-forming composition comprising nizatidine and a polymeric binder. Alternately, the nizatidine-containing core is produced by granulating and milling and/or by extruding and spheronizing a polymer composition containing nizatidine.

Oral administration is preferred. The dosage form may be administered two times a day, preferably once in the evening and once in the morning.

In embodiments, the invention is directed to a method of treating a human having a gastro-intestinal disorder, comprising administering to the human a pulsatile release pharmaceutical dosage form, preferably bi-modal, comprising a therapeutically effective amount of a histamine H₂ antagonist. Histamine H₂ antagonists suitable for the present invention include nizatidine, cimetidine, ranitidine and famotidine, preferably nizatidine. The gastro-intestinal disorder is preferably an acid peptic disease, such as Gastroesophogeal Reflux Disease (GERD), ulcers, heartburn, Nocturnal Acid Breakthrough, nighttime heartburn, regurgitation and retrosternal pain. Preferably about 100 mg to about 400 mg of a histamine H₂ antagonist is administered, more preferably about 150 mg to about 300 mg of a histamine H₂ antagonist is administered. In some embodiments, the human subject is diabetic.

In embodiments, the invention is directed to a method of treating a human having a gastro-intestinal disorder, comprising administering to the human once daily a bi-modal pulsatile release oral pharmaceutical dosage form comprising: immediate release (IR) beads comprising a core particle containing nizatidine and timed pulsatile release (TPR) beads, wherein said TPR beads comprise a core particle containing nizatidine and a pulse coating surrounding said core. Preferably, about 150 mg to about 300 mg of nizatidine is administered.

In embodiments, the invention is directed to a method of administering nizatidine, comprising administering orally to a human a bi-modal pulsatile release formulation comprising nizatidine that provides two peak blood plasma concentrations of nizatidine occurring from about 2.0 to about 4.0 hours apart, more preferably about 3.5 to about 4.0 hours apart, wherein the first peak concentration occurs within 2 hours after administration, preferably within 1 hour after administration, and wherein a therapeutic level of nizatidine is maintained for about 6 to about 8 hours after administration. The formulation preferably comprises immediate release (IR) beads comprising a core particle containing nizatidine and timed pulsatile release (TPR) beads, wherein said TPR beads comprise a core particle containing nizatidine and a pulse coating surrounding said core. The formulation is preferably administered once or twice a day, in the evening and/or in the morning. Preferably, about 150 mg to about 300 mg of nizatidine is administered.

Where 150 mg of nizatidine is administered, the first peak concentration is preferably from about 200 to about 800 ng/ml, more preferably from about 300 to about 700 ng/mL, most preferably from about 350 to about 600 ng/mL, and/or the second peak concentration is preferably from about 200 to about 800 ng/ml, more preferably from about 400 to about 800 ng/mL, most preferably from about 500 to about 700 ng/mL. Where 300 mg of nizatidine is administered, the first peak concentration is preferably from about 400 to about 1000 ng/ml, more preferably from about 500 to about 900 ng/ml, most preferably from about 600 to about 800 ng/mL, and/or the second peak concentration is preferably from about 600 to about 1200 ng/ml, more preferably from about 700 to about 1100 ng/mL, most preferably from about 800 to about 1000 ng/mL.

In embodiments, the invention is directed to a method of administering nizatidine, comprising administering orally to a human a bi-modal pulsatile release formulation producing a first peak blood plasma concentration and a second peak blood level concentration, wherein the ratio of the first peak to the second peak is between about 75:25 and about 25:75, more preferably between about 67:33 and about 33:67. Even more preferably, the ratio is about 50:50.

The formulation of the embodiment preferably comprises immediate release (IR) beads comprising a core particle containing nizatidine, timed pulsatile release (TPR) beads, wherein said TPR beads comprise a core particle containing nizatidine, and a pulse coating surrounding said core. The formulation is administered once or twice a day, in the evening and/or in the morning. Preferably, about 150 mg to about 300 mg of nizatidine is administered.

When about 150 mg of nizatidine is administered, the first peak concentration is preferably from about 200 to about 800 ng/ml, more preferably from about 300 to about 700 ng/mL, most preferably from about 350 to about 600 ng/mL and/or the second peak concentration is preferably from about 200 to about 800 ng/ml, more preferably from about 300 to about 700 ng/mL, most preferably from about 350 to about 600 ng/mL. When about 300 mg of nizatidine is administered, the first peak concentration is preferably from about 400 to about 1000 ng/ml, more preferably from about 500 to about 900 ng/mL, most preferably from about 600 to about 800 ng/ml, and/or the second peak concentration is preferably from about 600 to about 1200 ng/ml, more preferably from about 700 to about 1100 ng/mL, most preferably from about 800 to 1000 ng/mL.

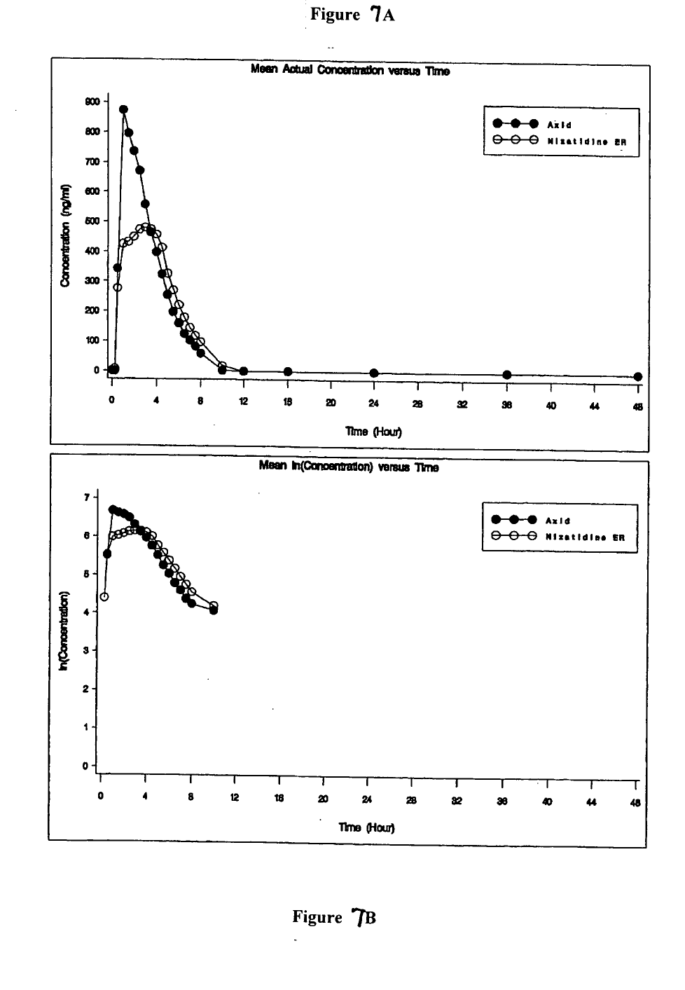

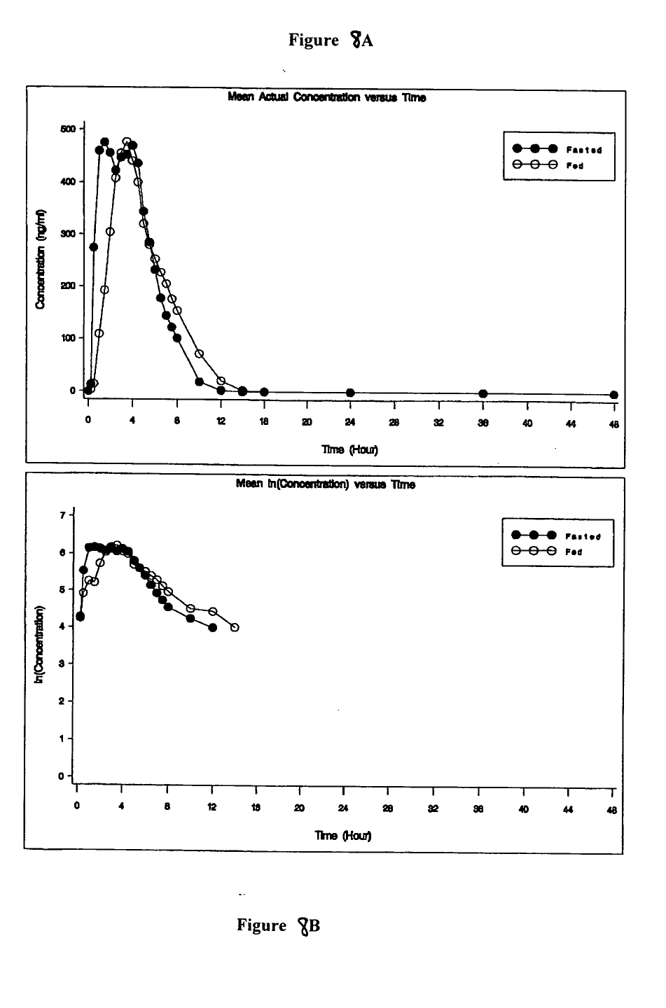

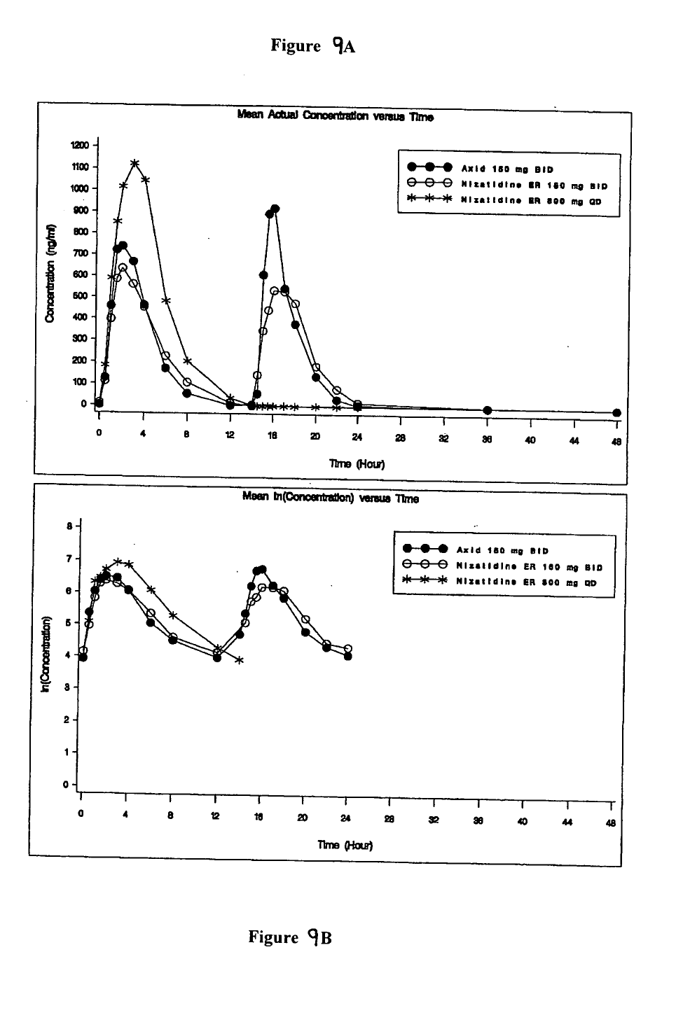

In embodiments, the invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 7.

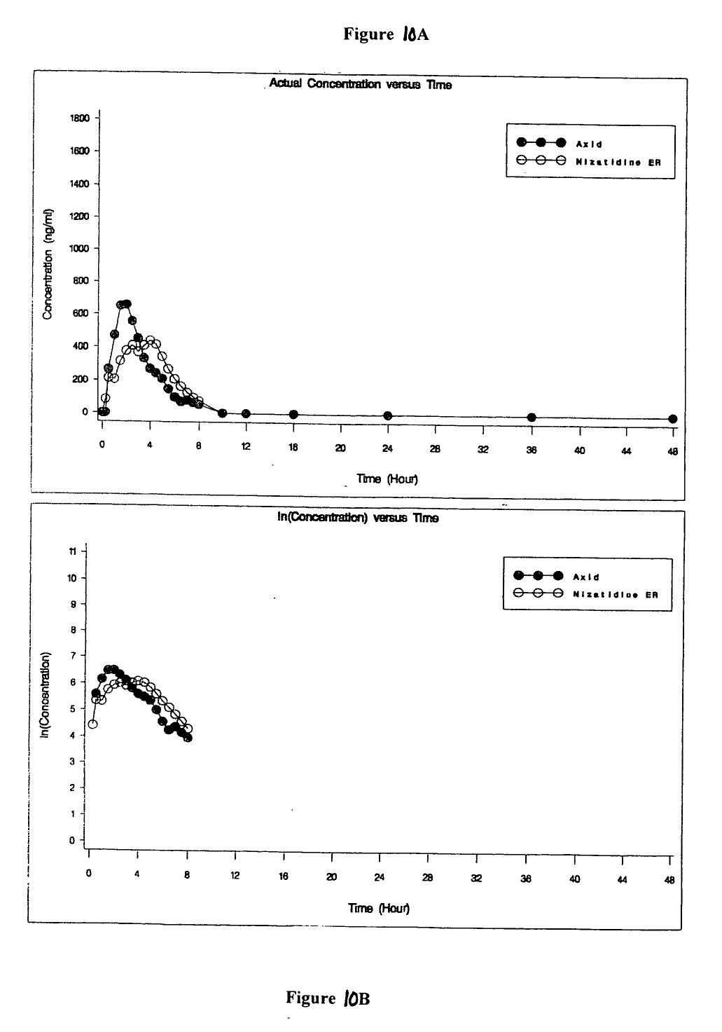

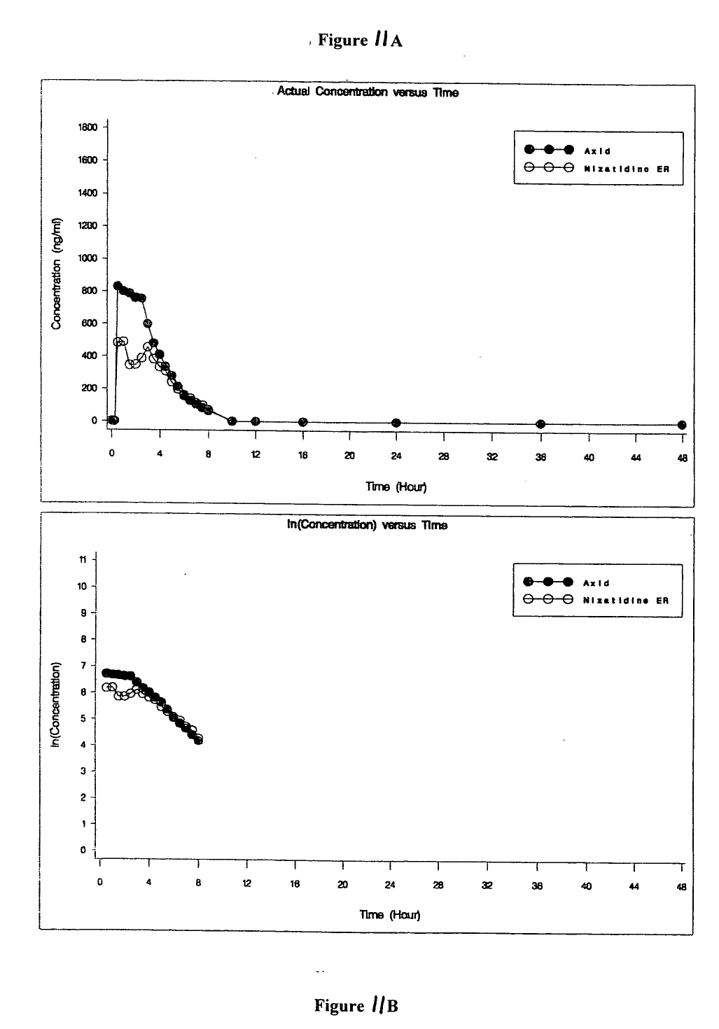

In embodiments, the invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 8.

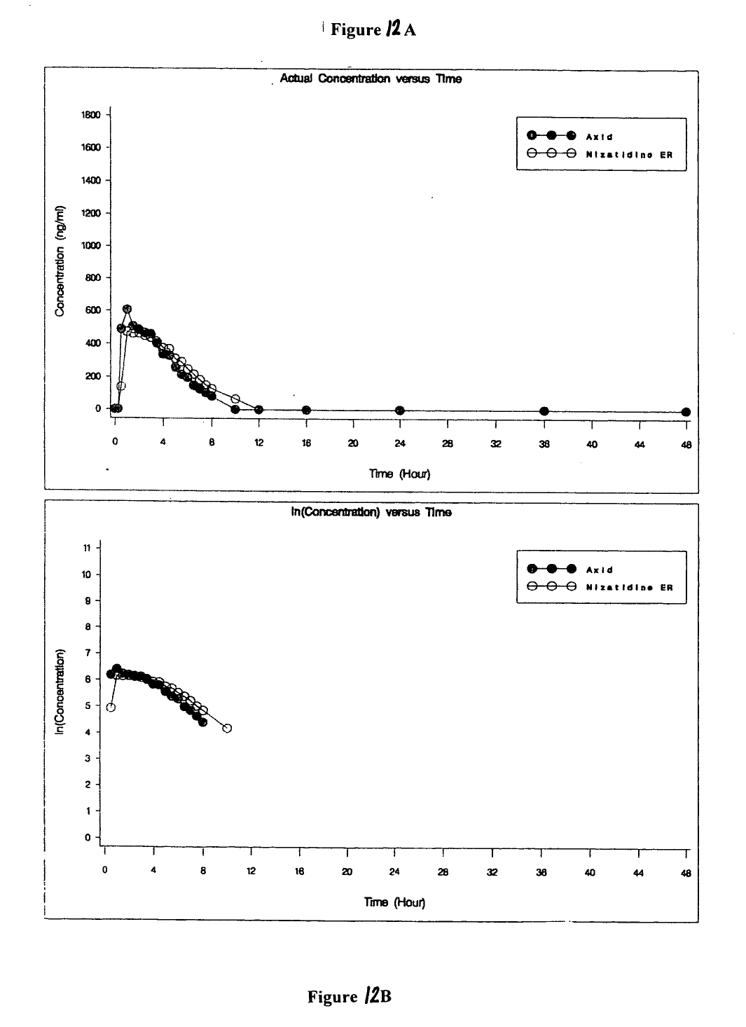

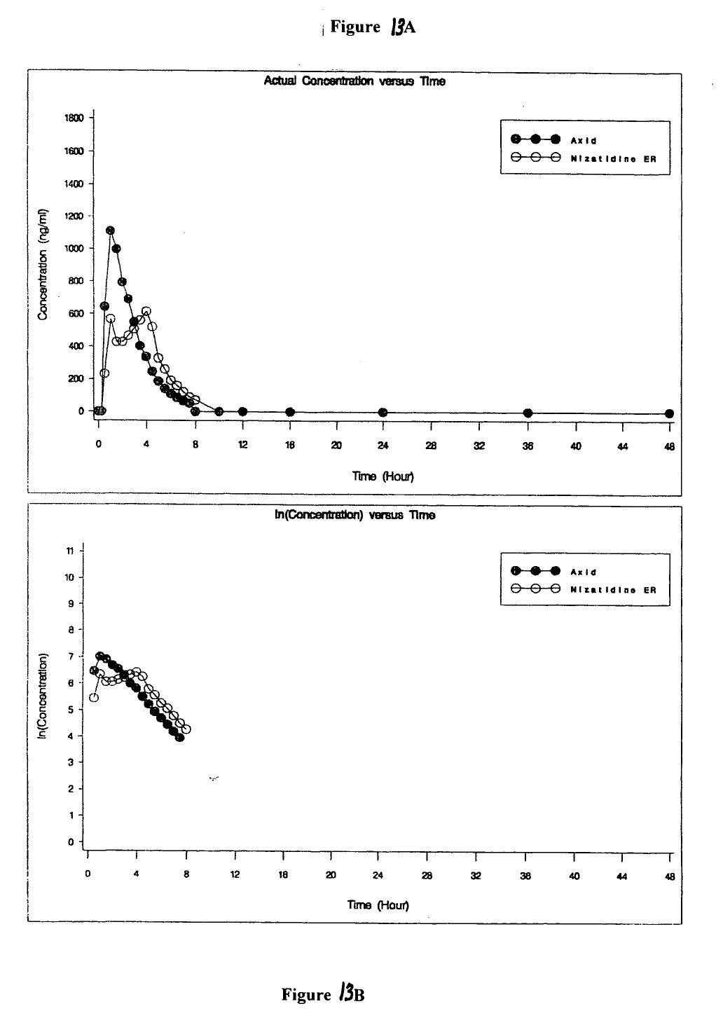

In embodiments, the invention is directed to an extended release (ER) oral dosage form comprising 150 mg of nizatidine, which after oral administration of a single one of said dosage forms twice daily in an adult human produces a blood plasma concentration of nizatidine ranging from 80 to 120% of the blood plasma concentration values per time period over the first twenty four hours shown in FIG. 9.

In embodiments, the invention is directed to an extended release (ER) oral dosage form comprising 150 mg of nizatidine, which after oral administration of two of said dosage forms once daily in an adult human produces a blood plasma concentration of nizatidine ranging from 80 to 120% of the blood plasma concentration values per time period over the first eight hours shown in FIG. 9.

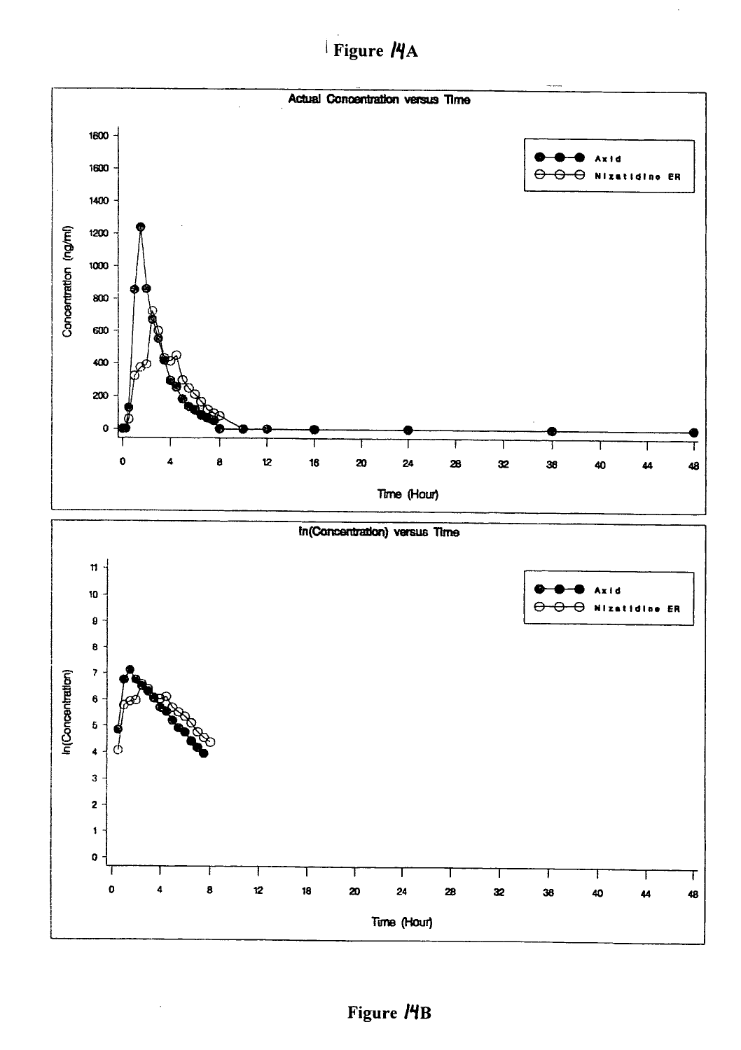

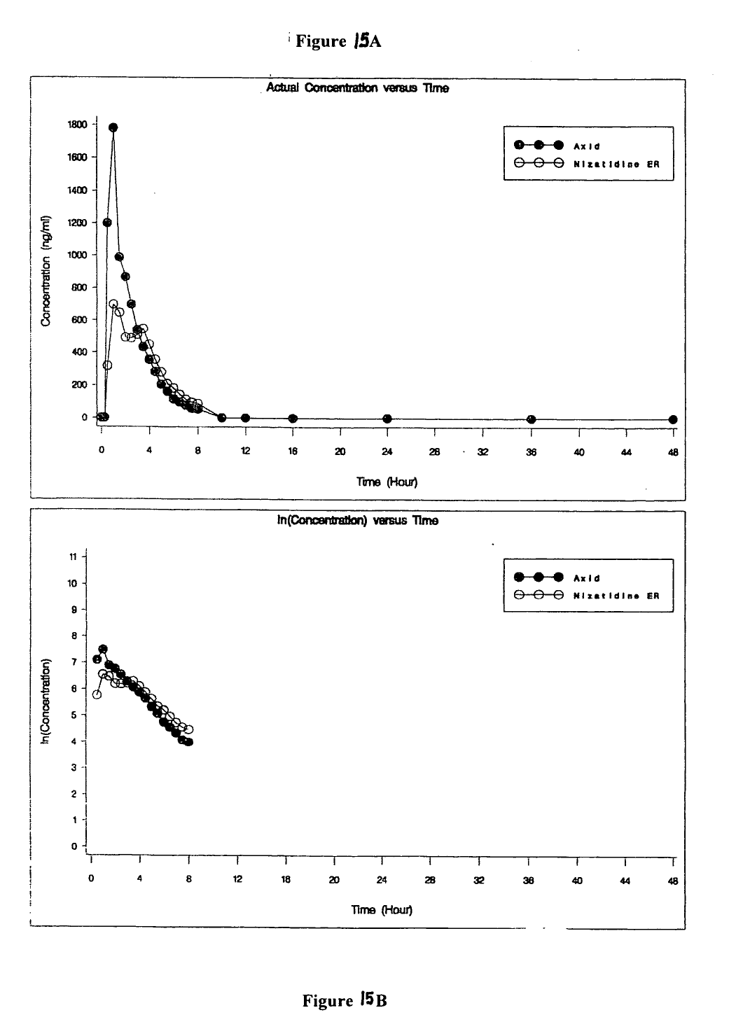

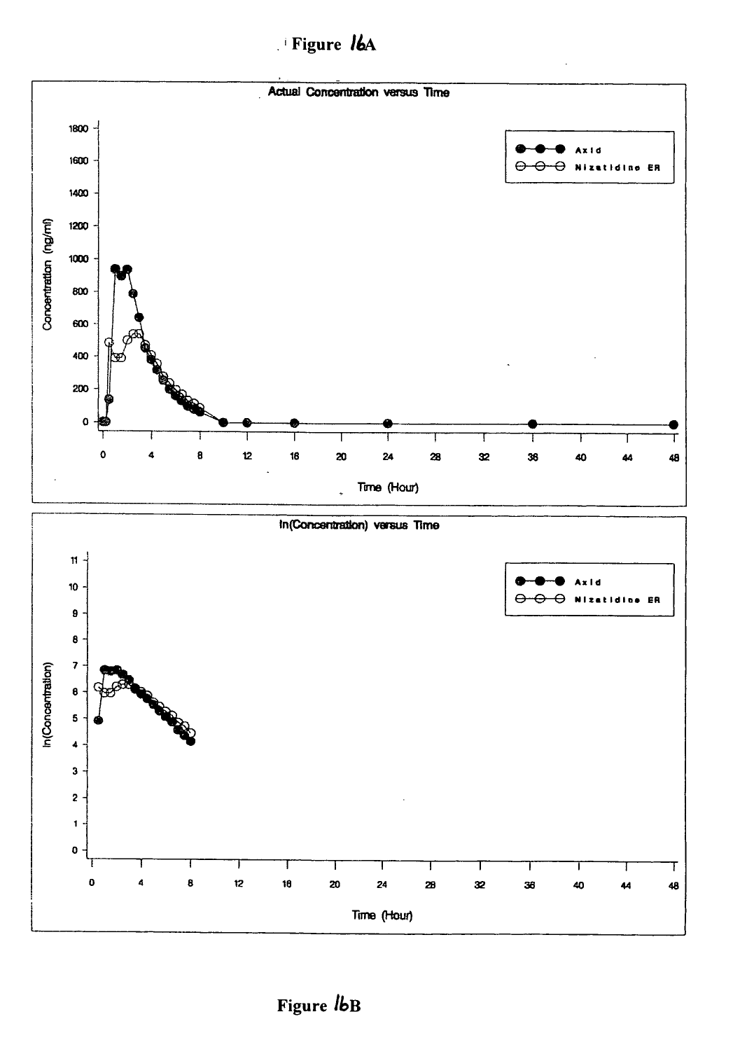

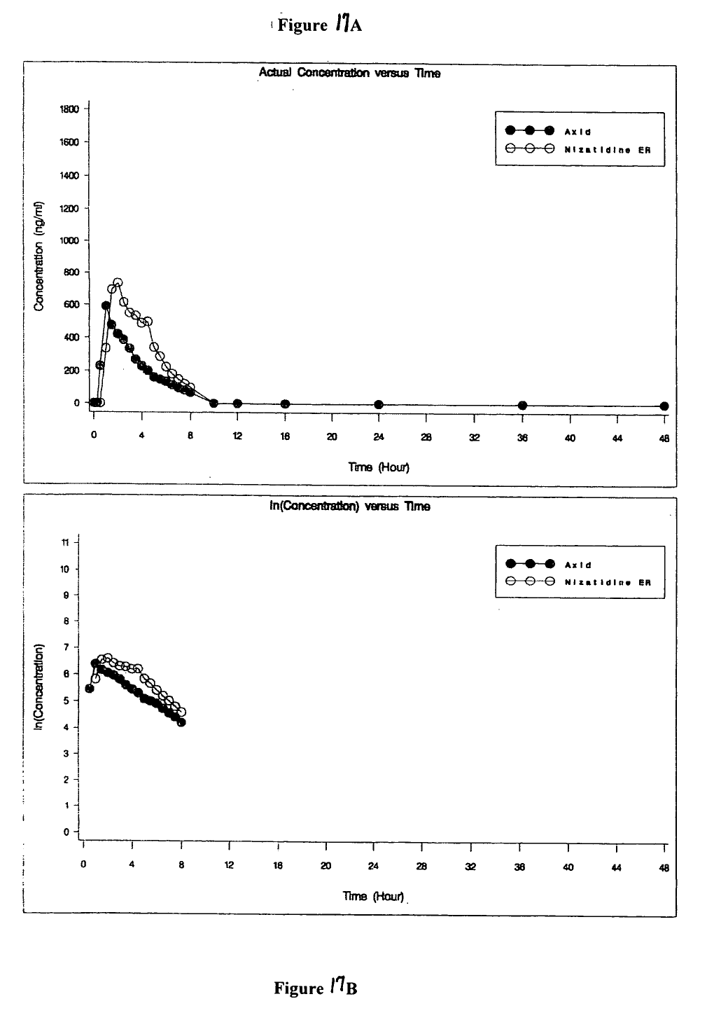

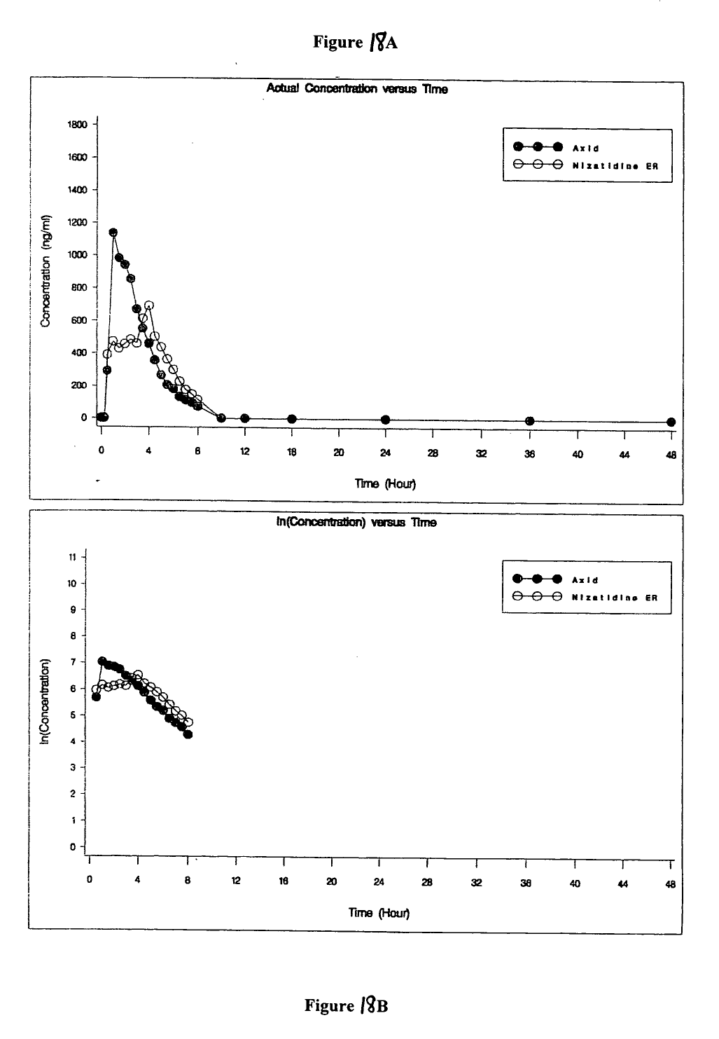

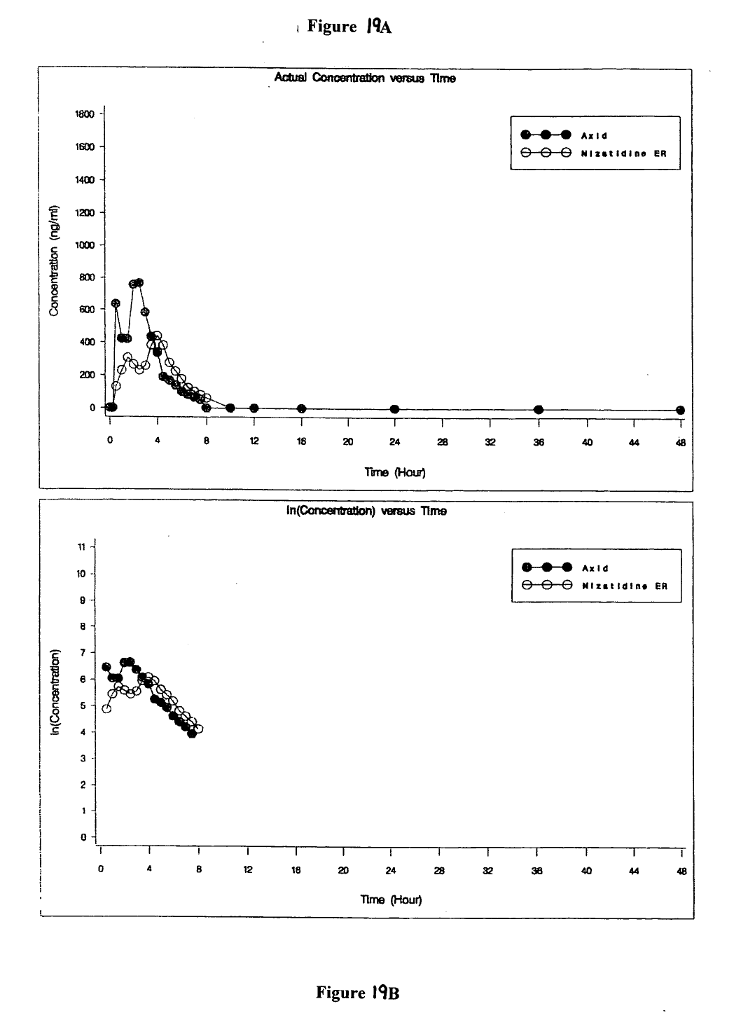

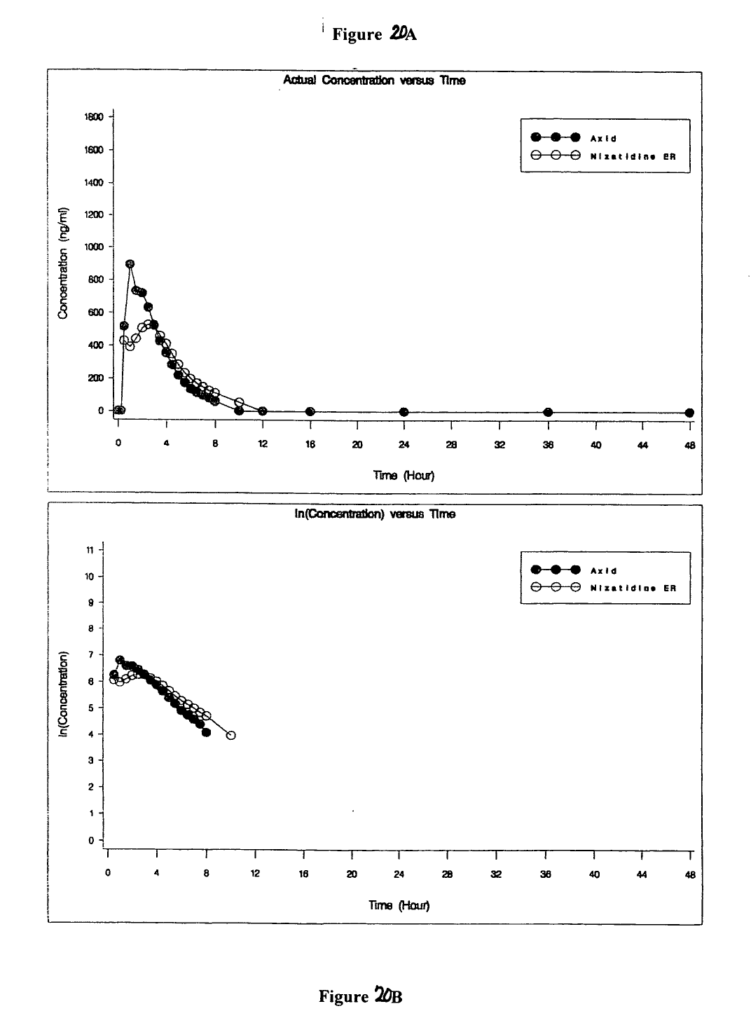

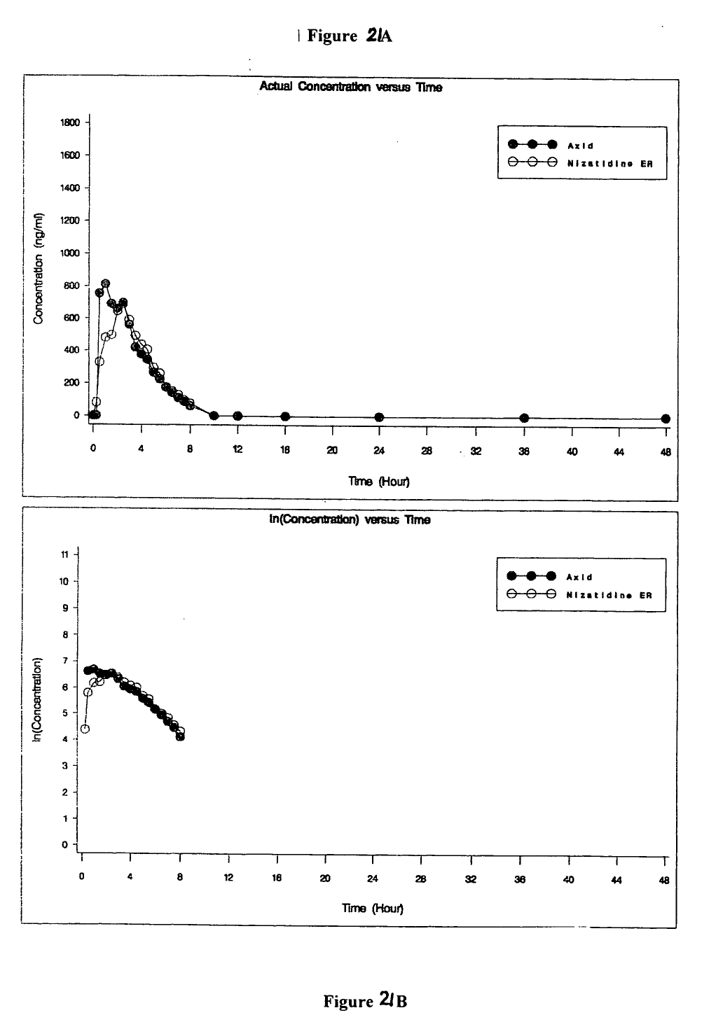

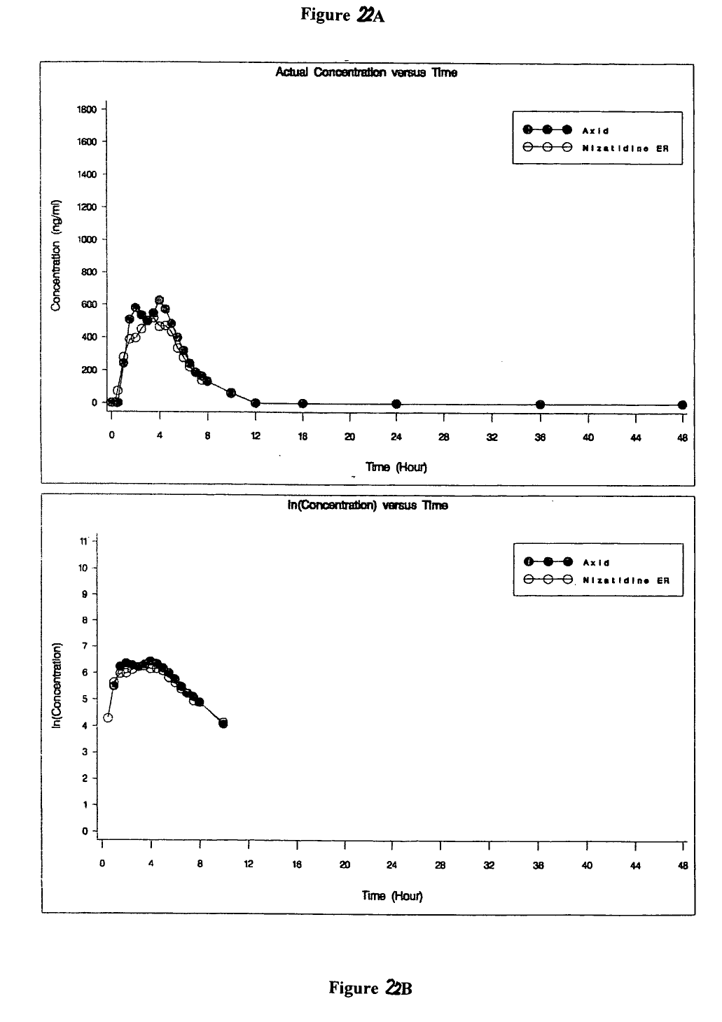

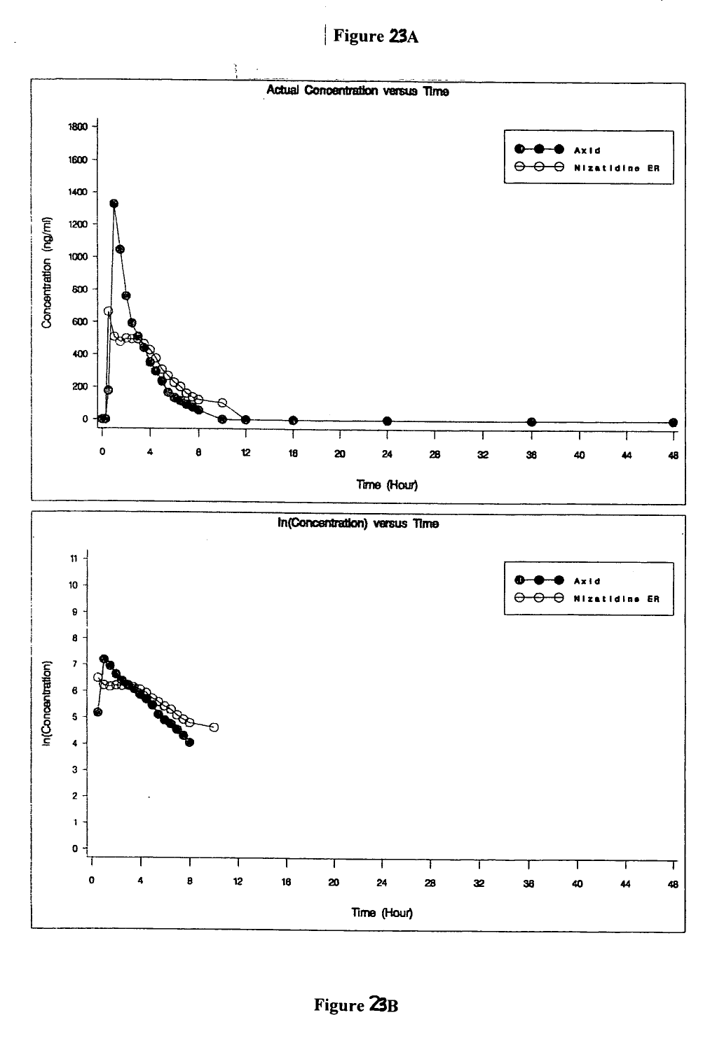

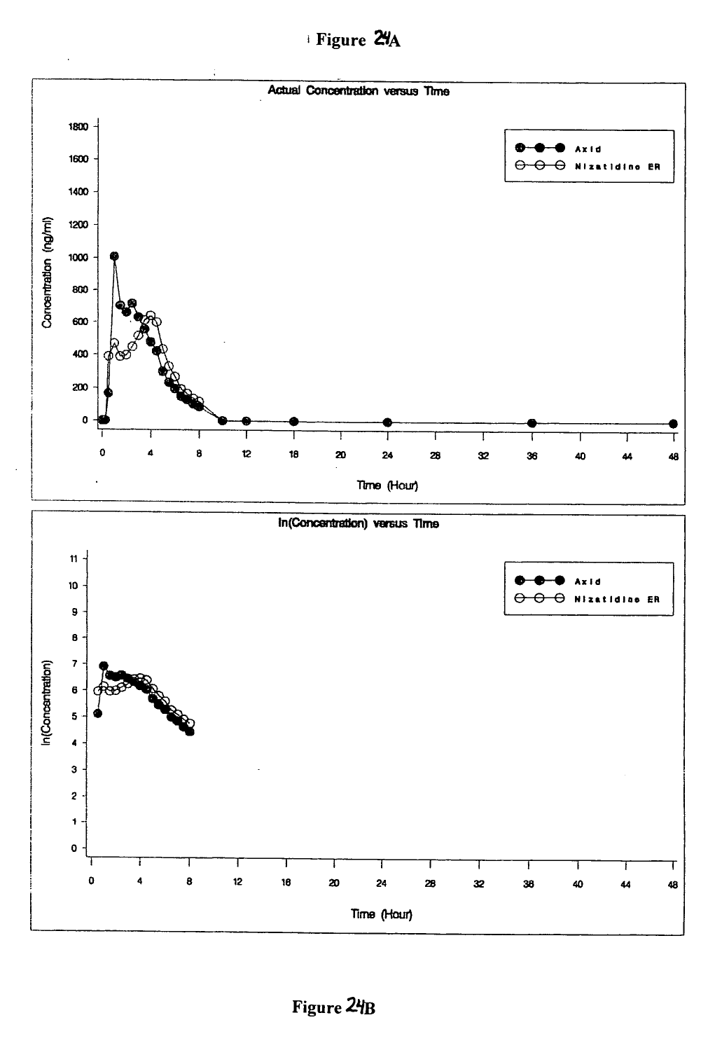

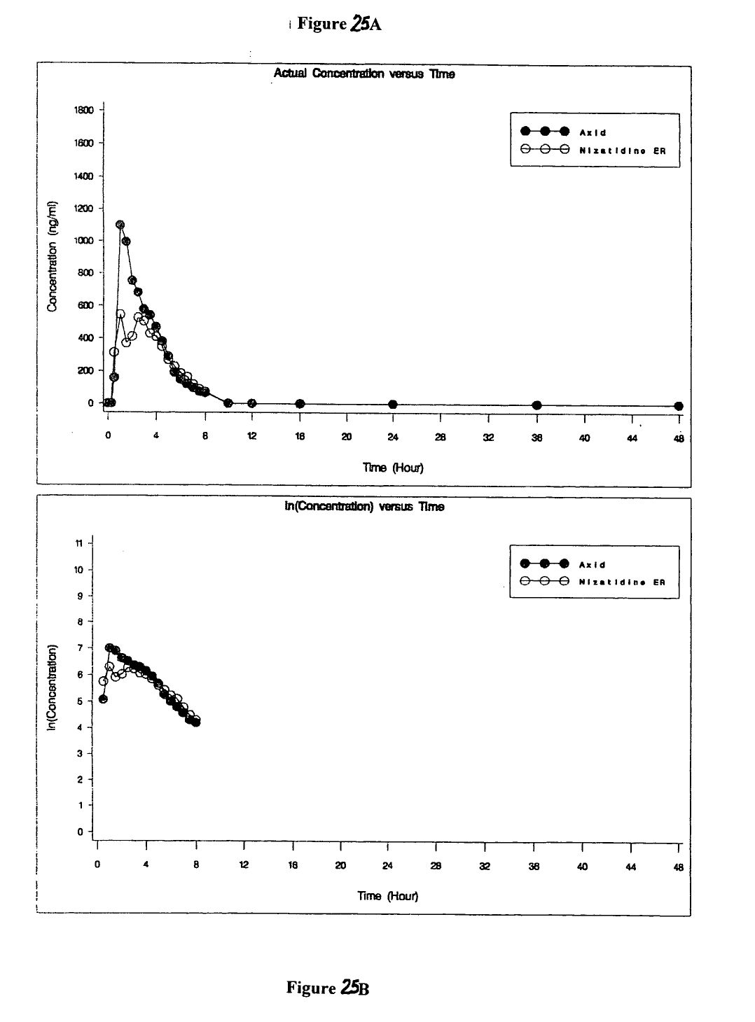

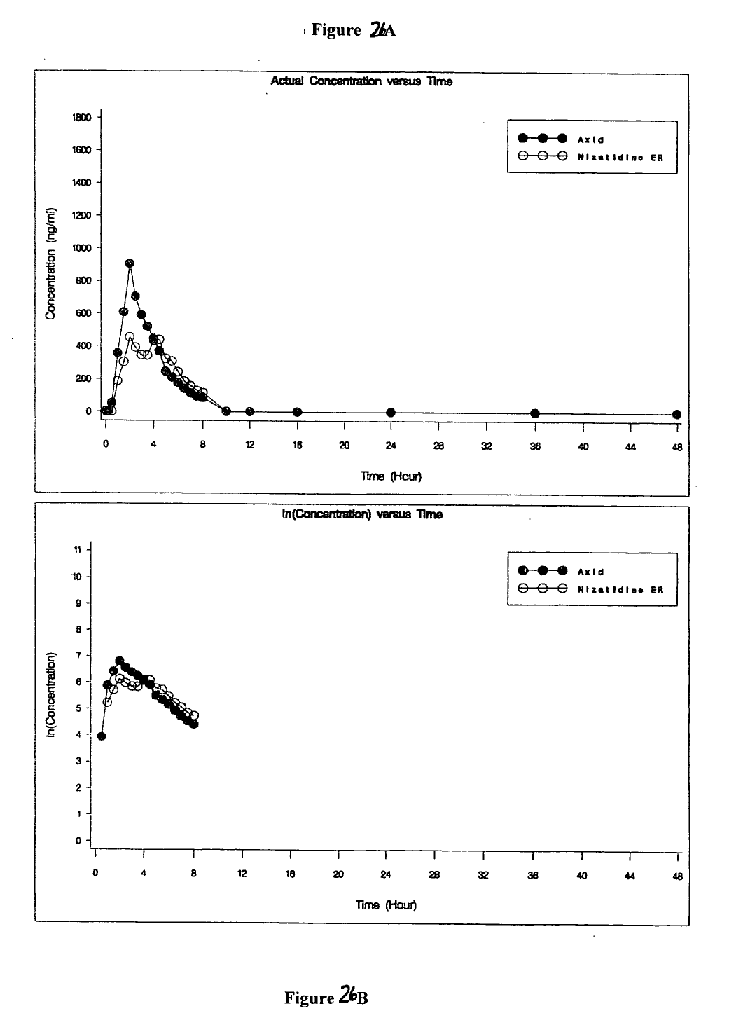

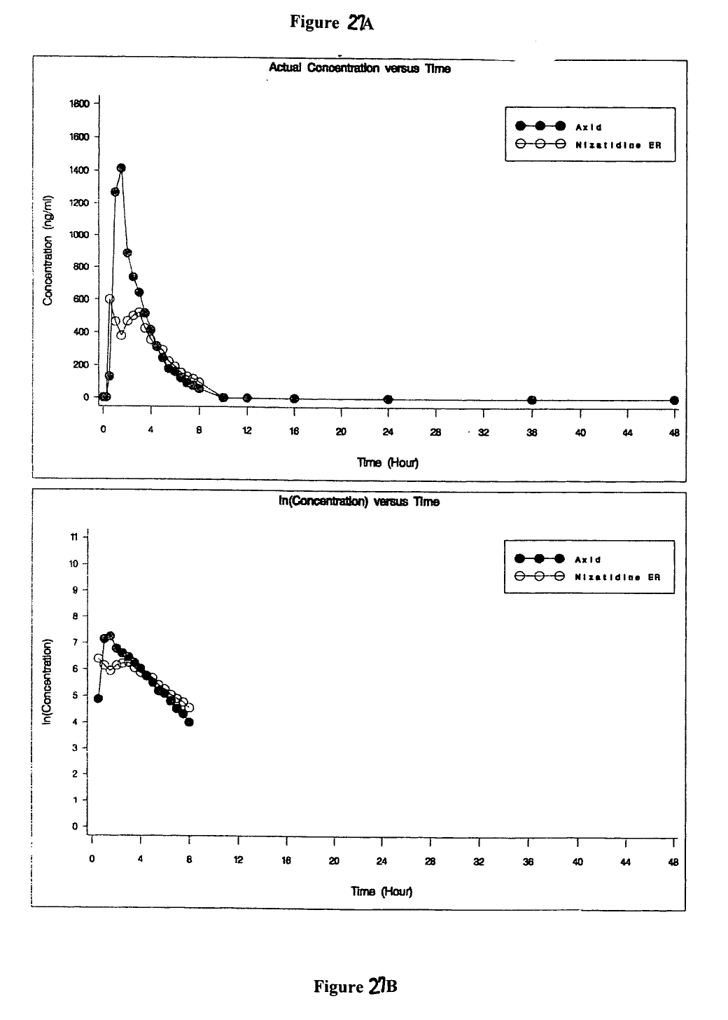

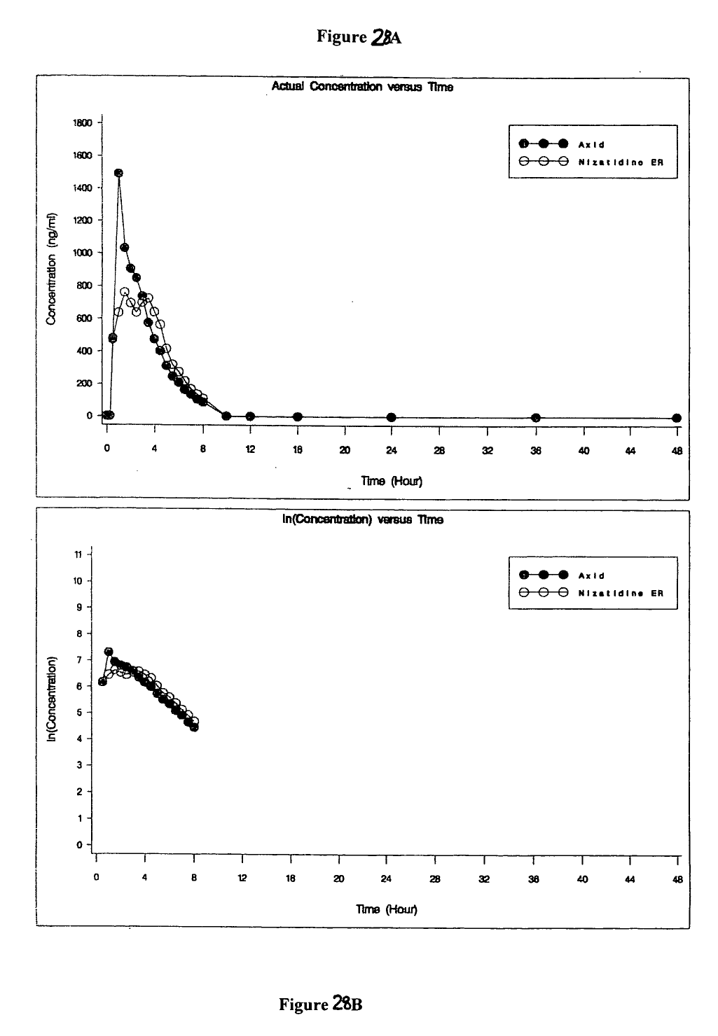

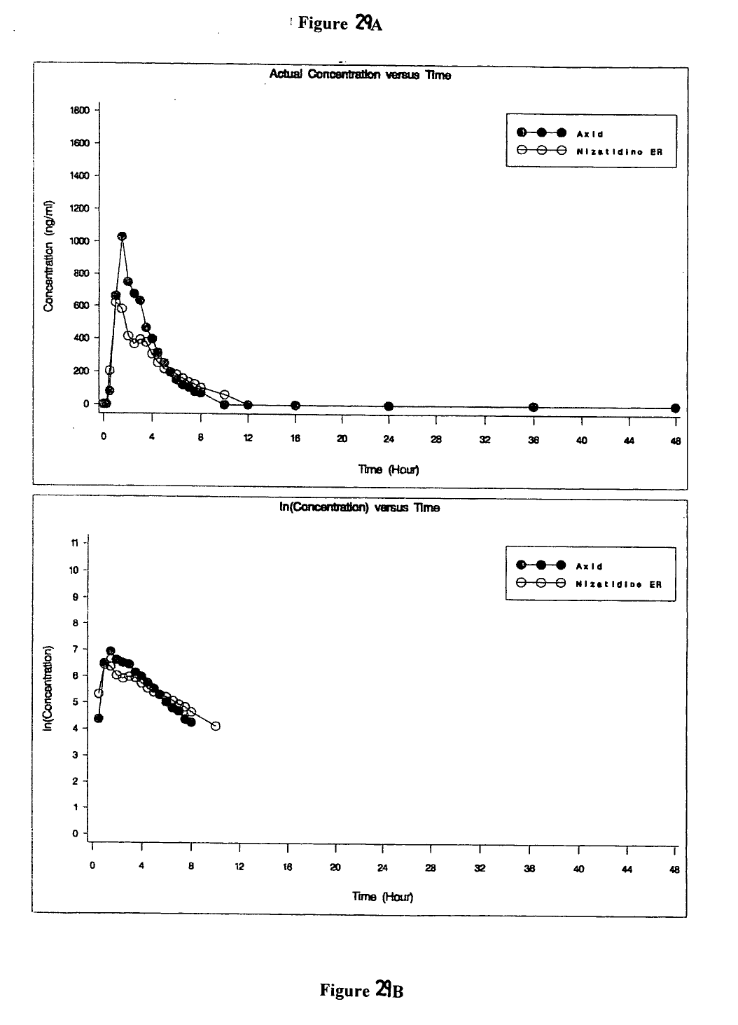

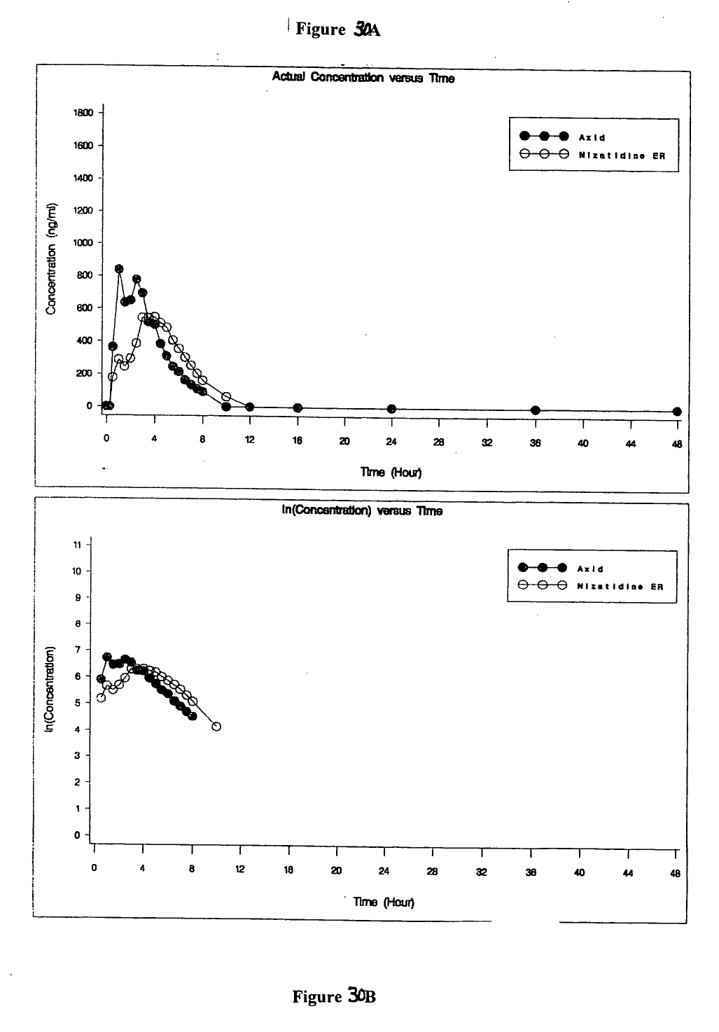

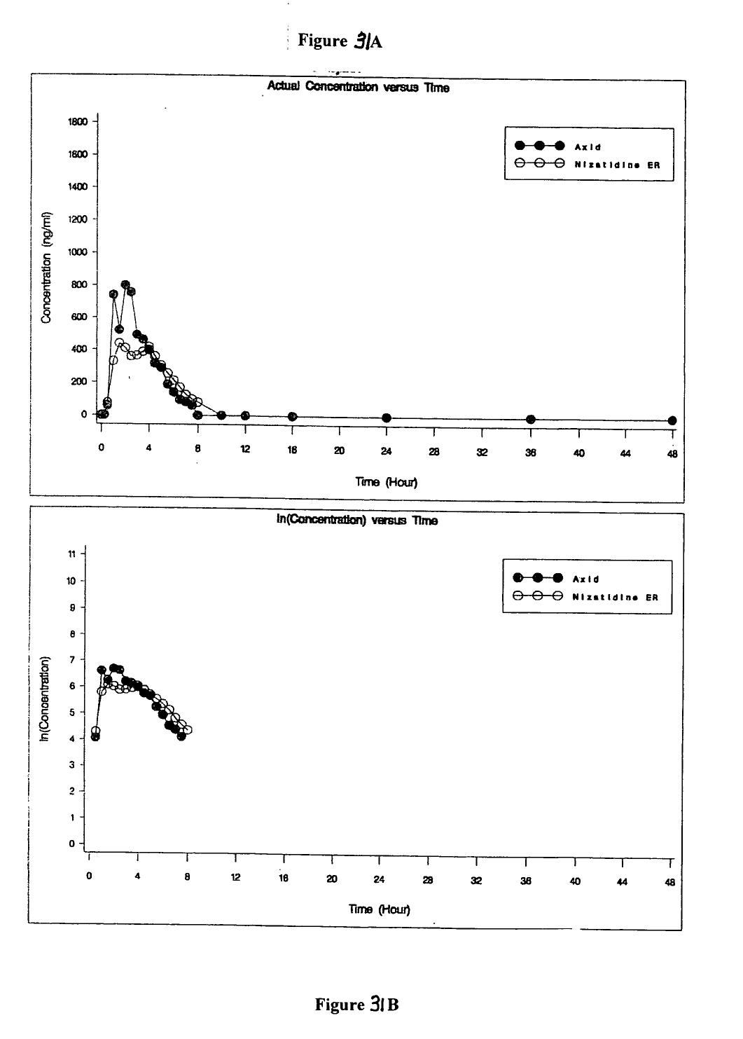

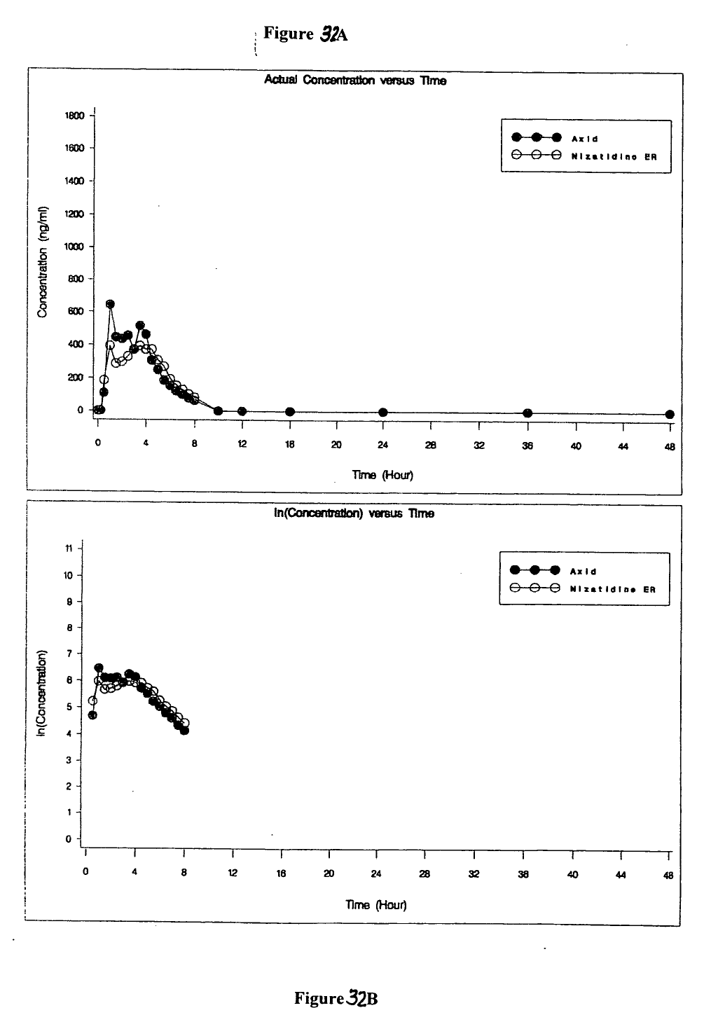

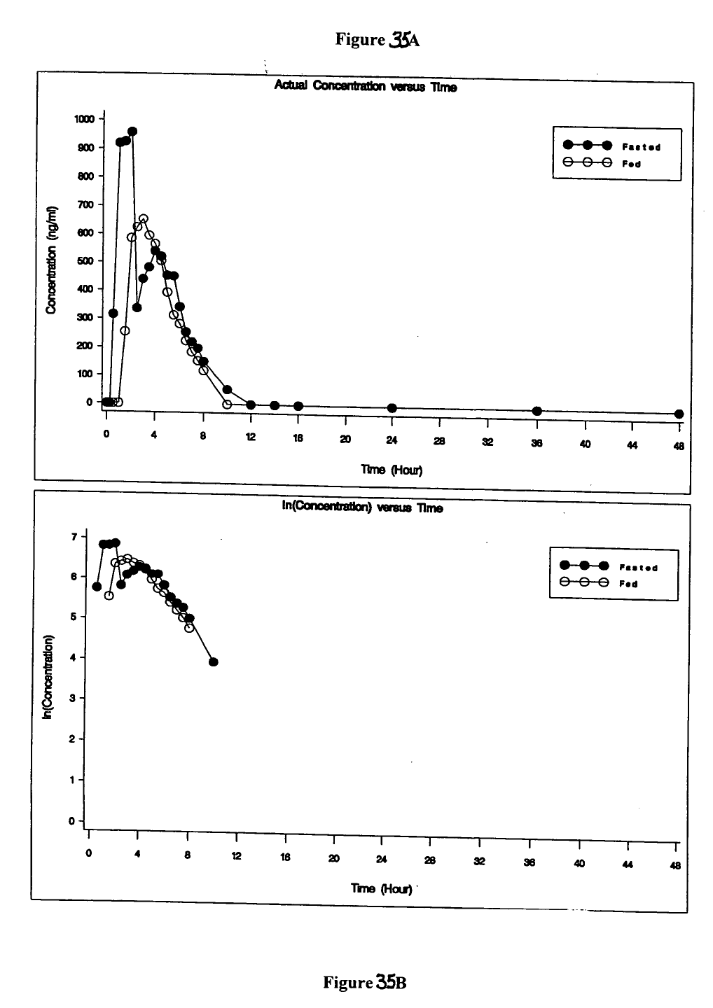

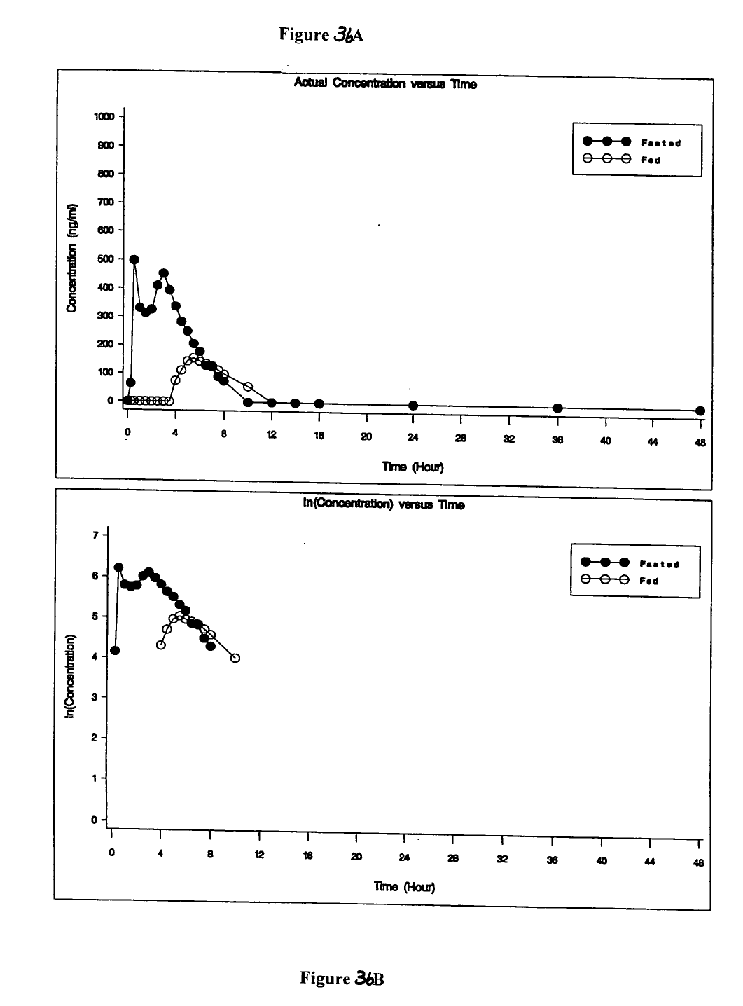

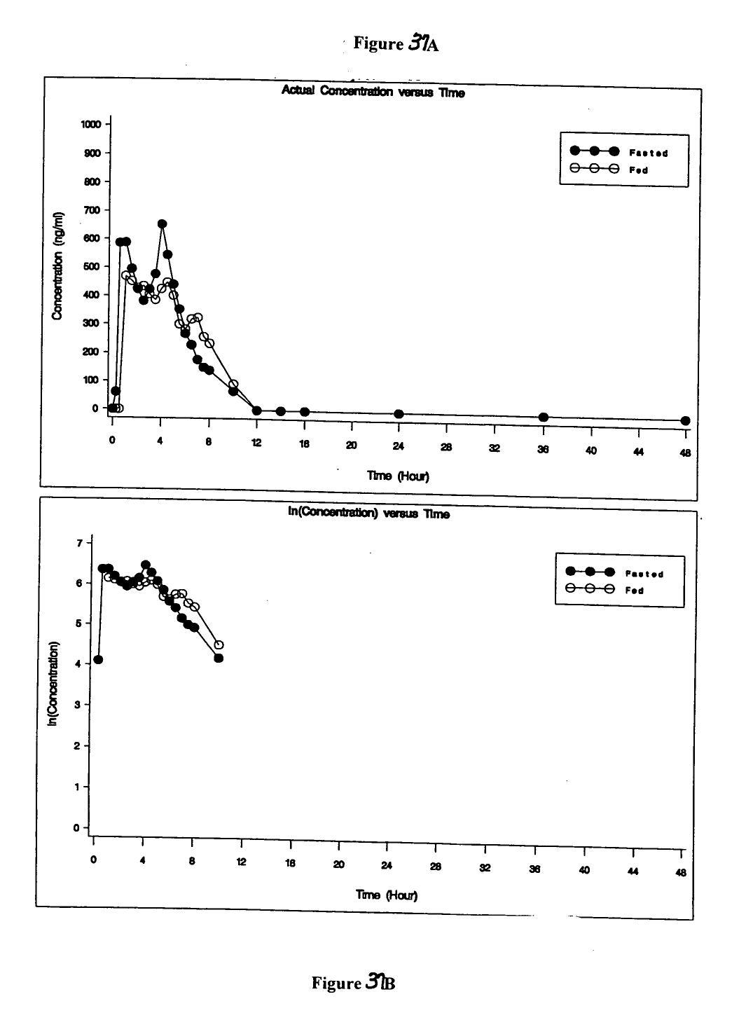

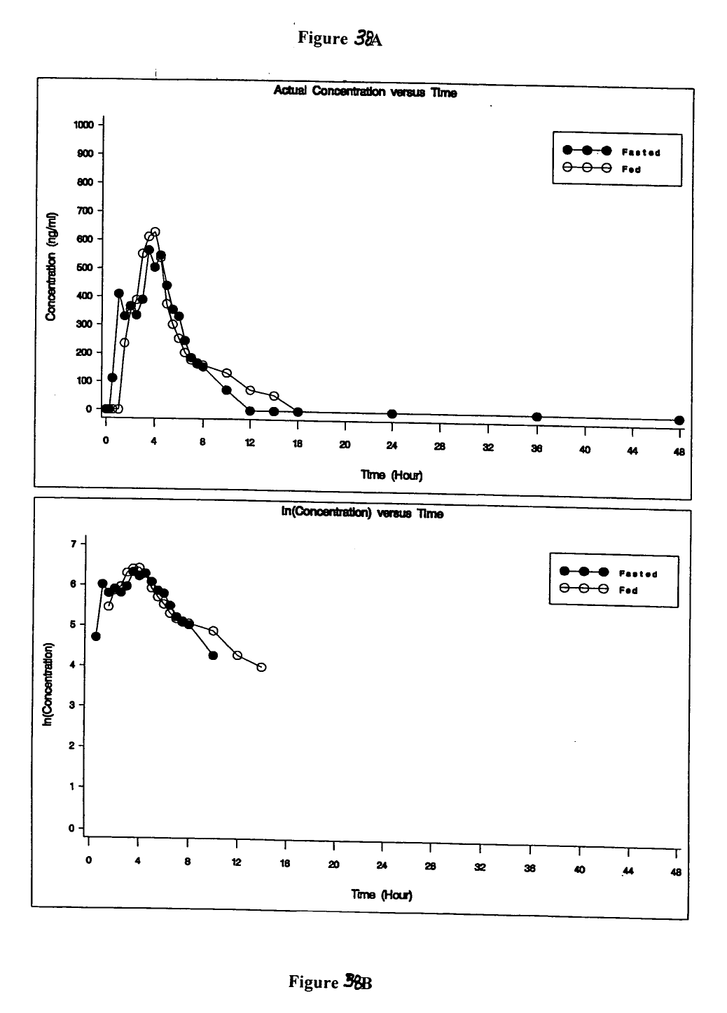

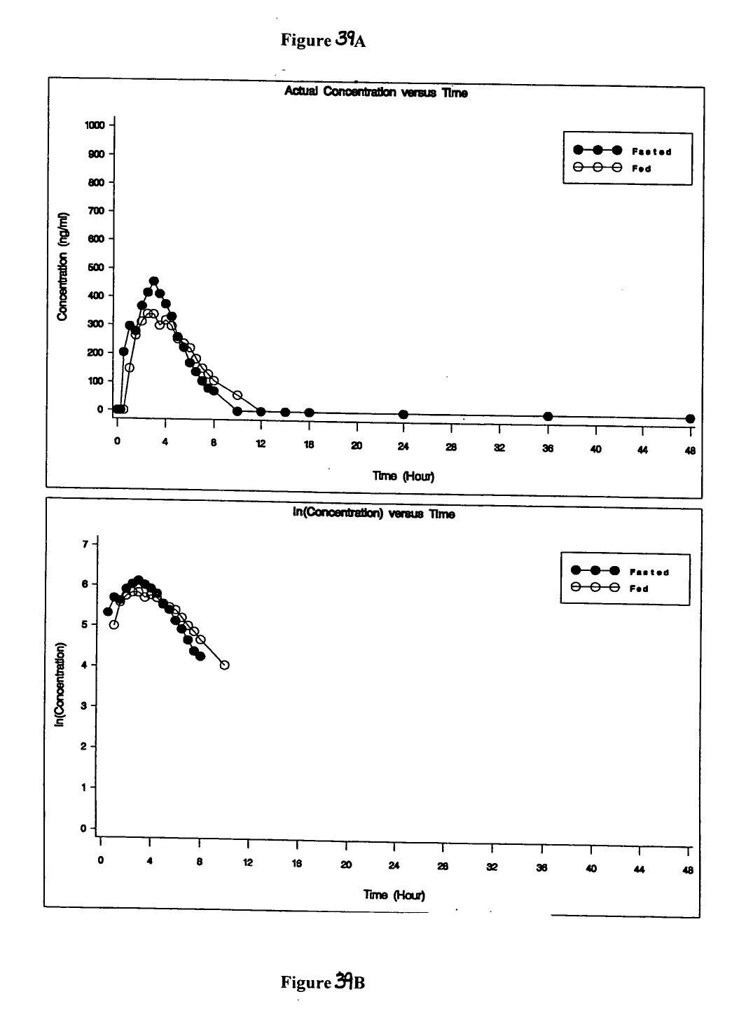

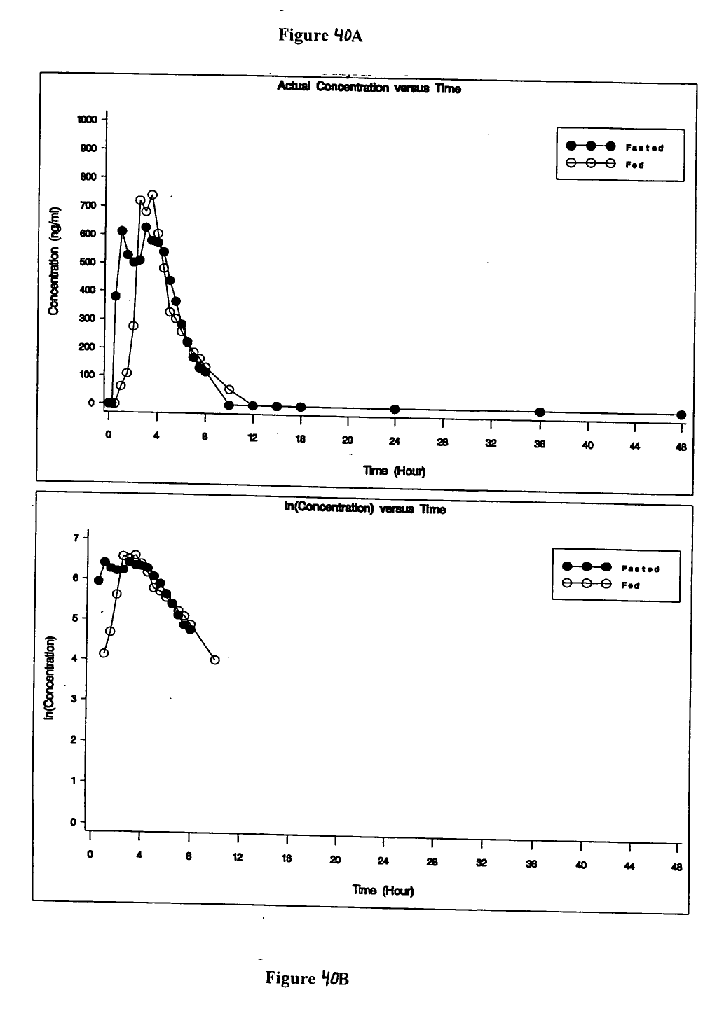

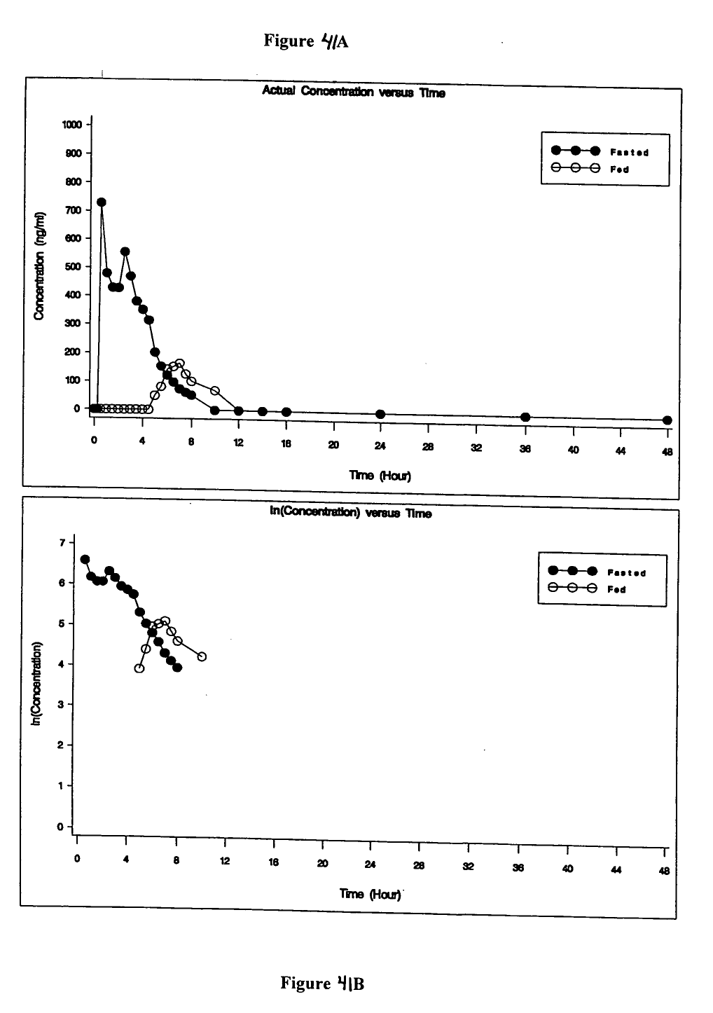

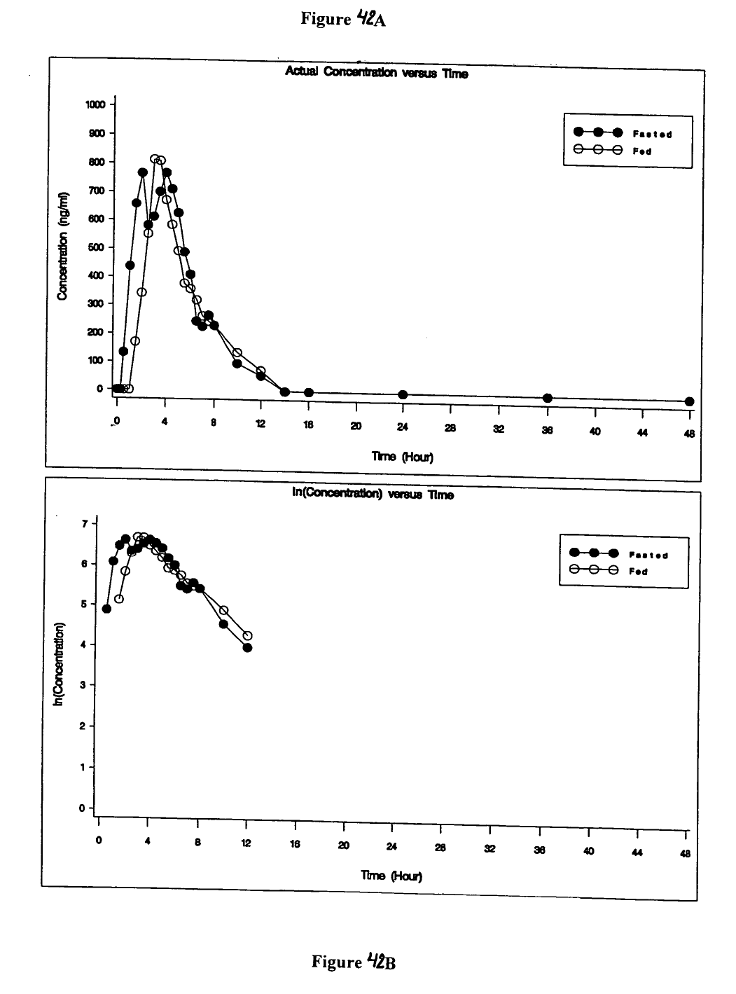

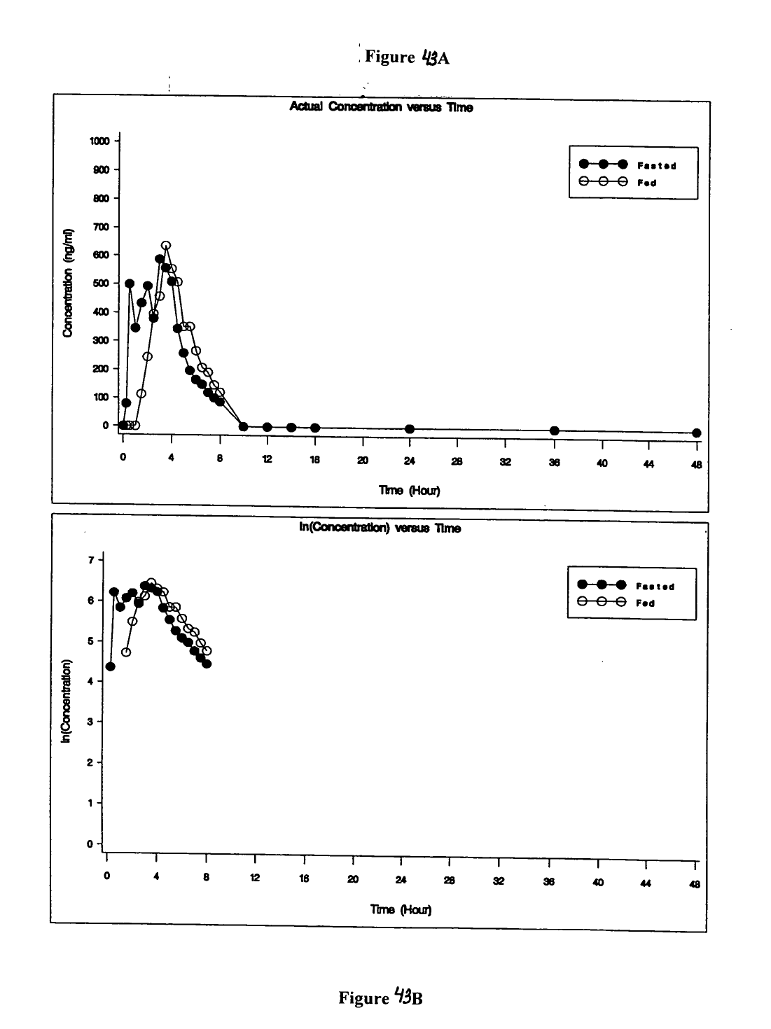

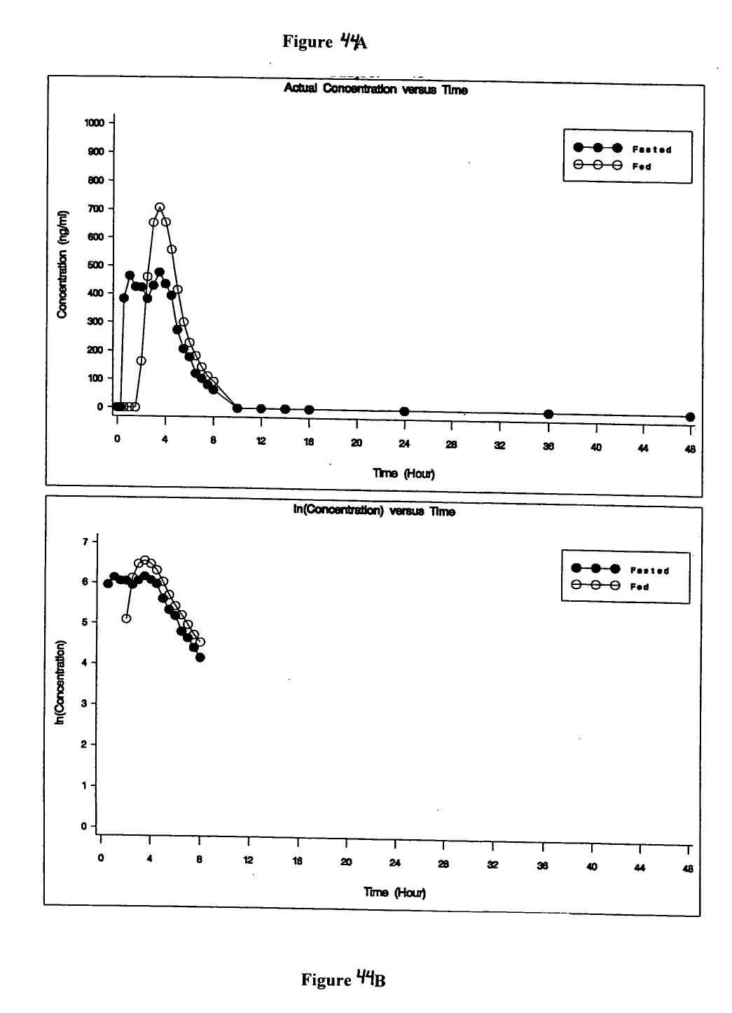

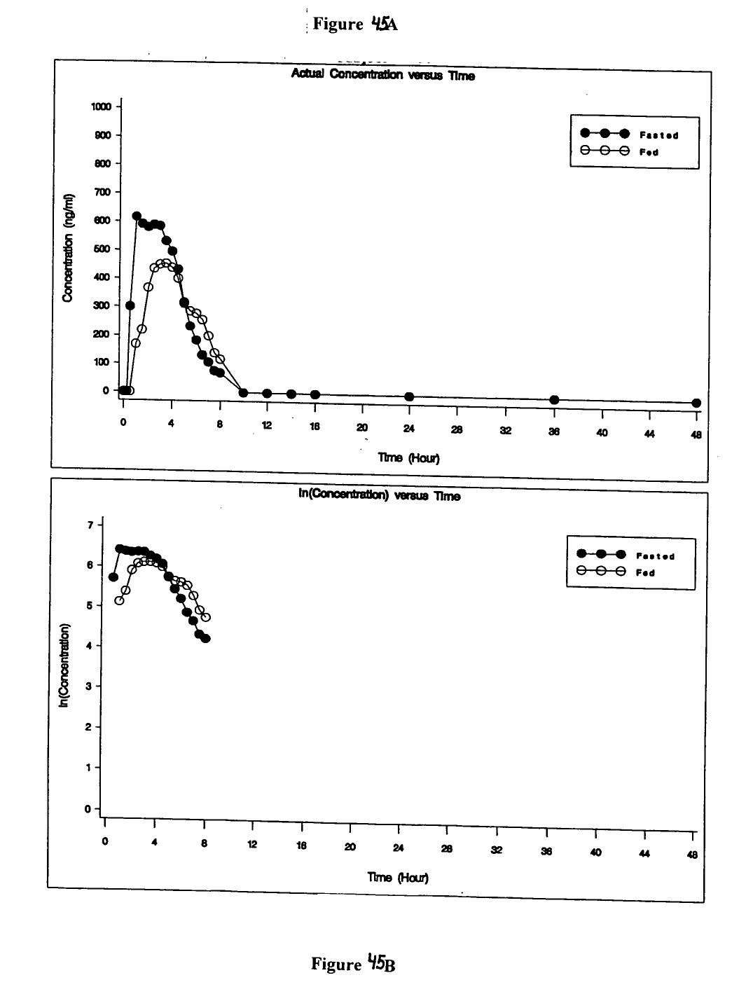

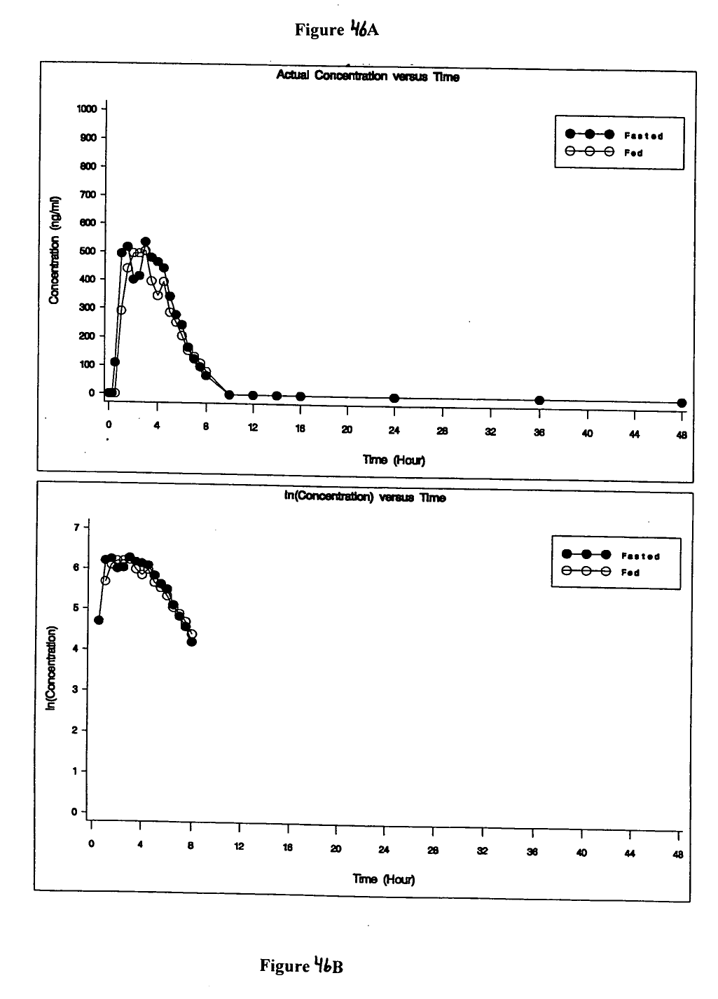

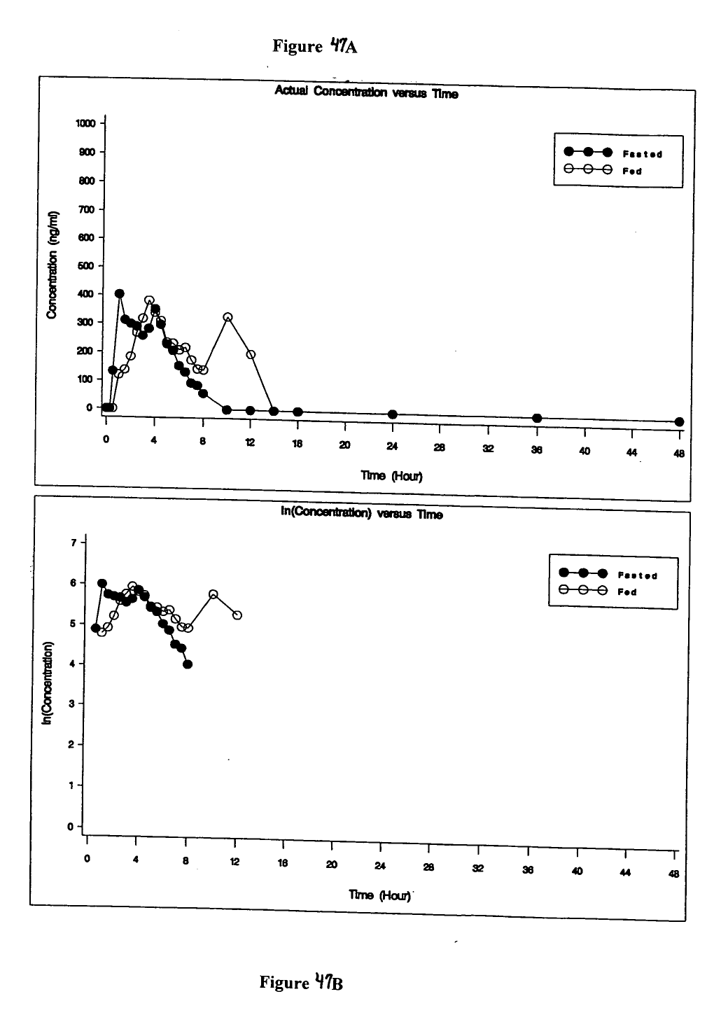

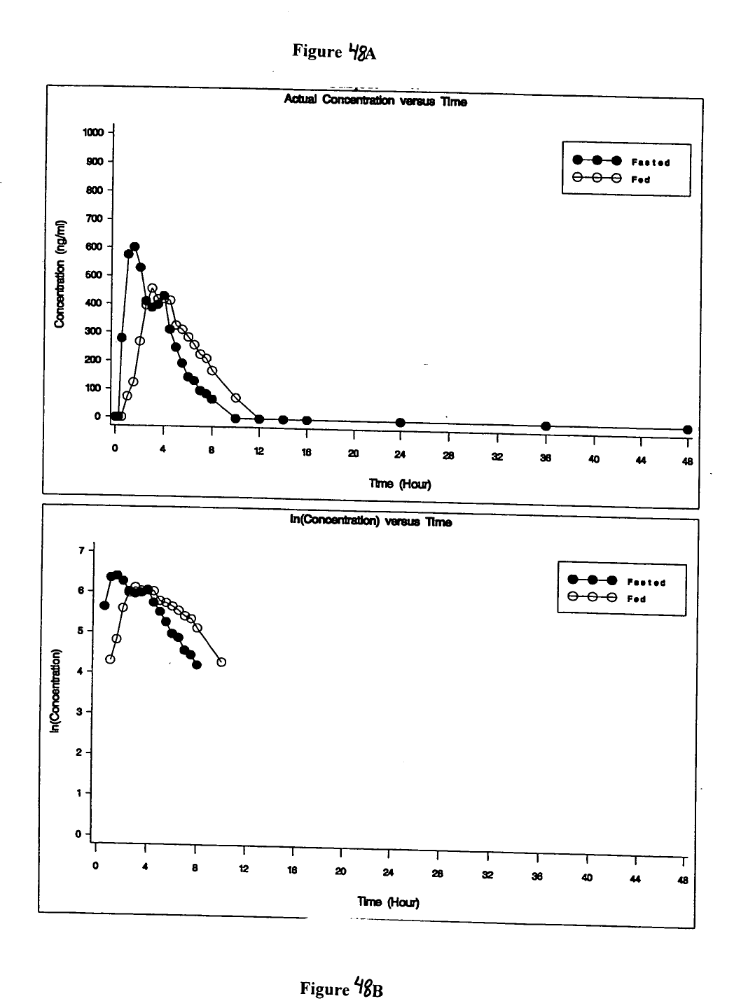

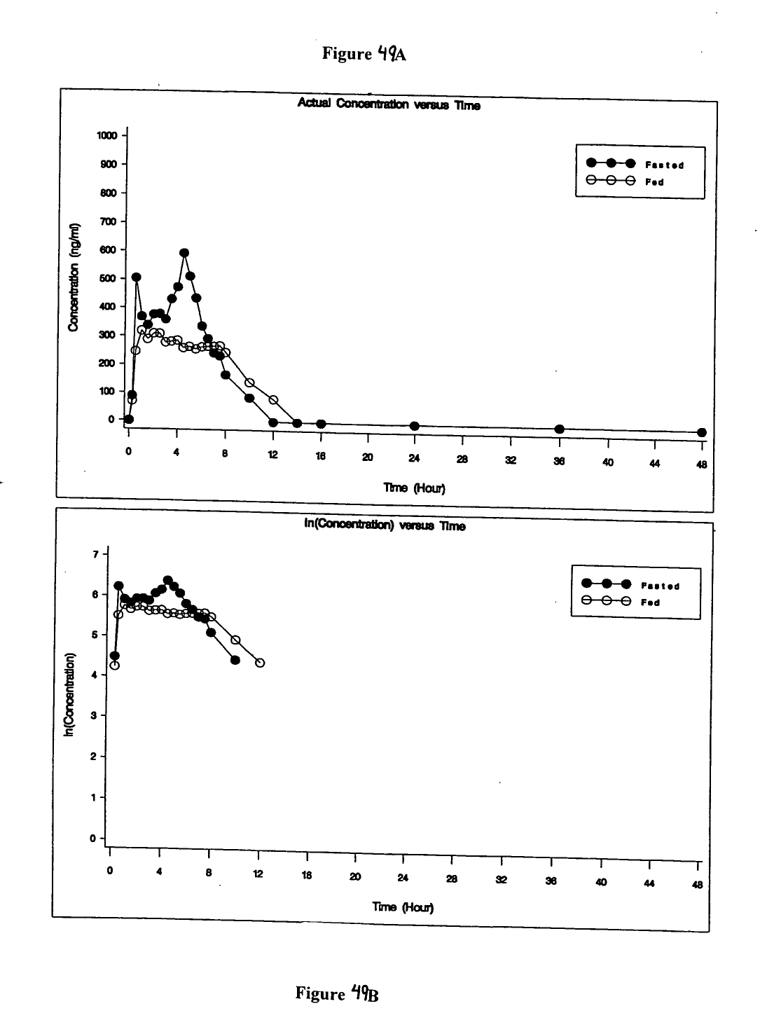

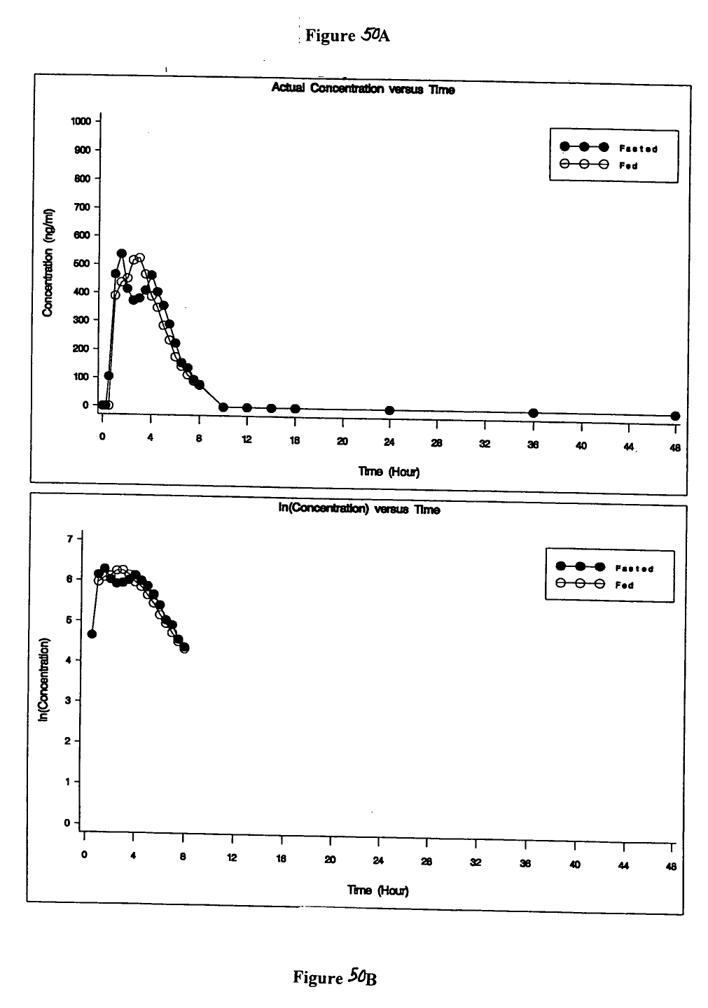

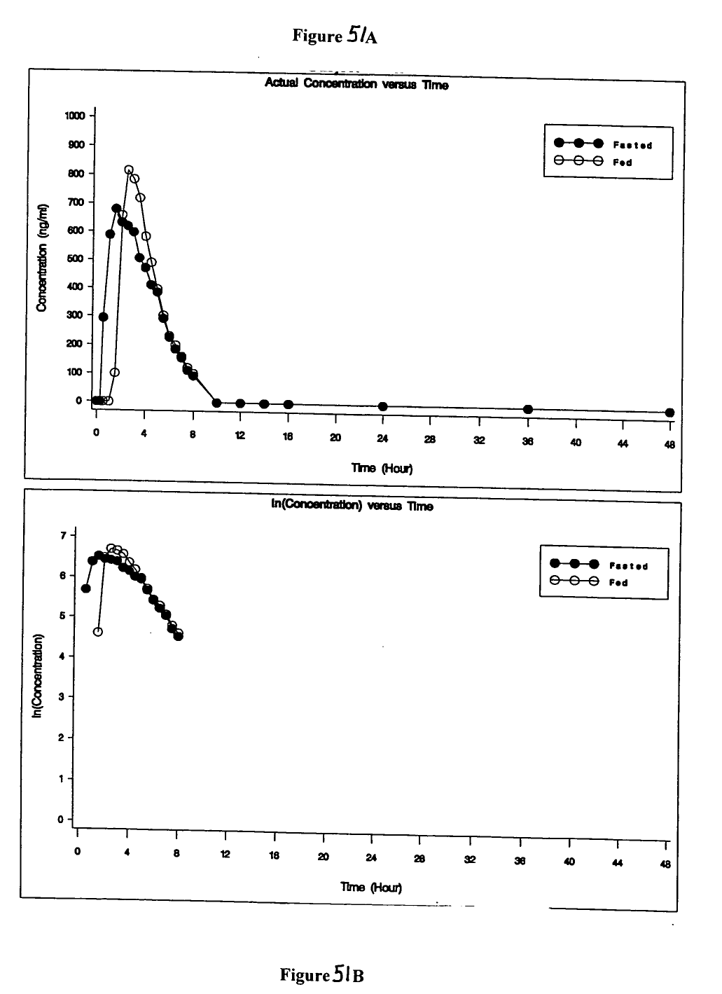

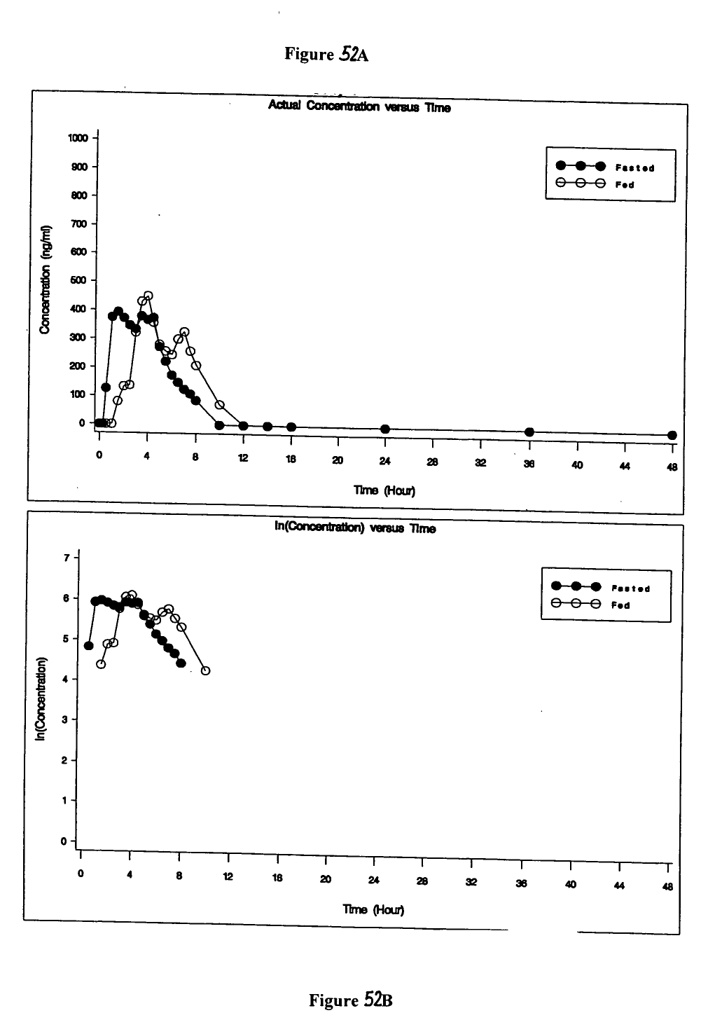

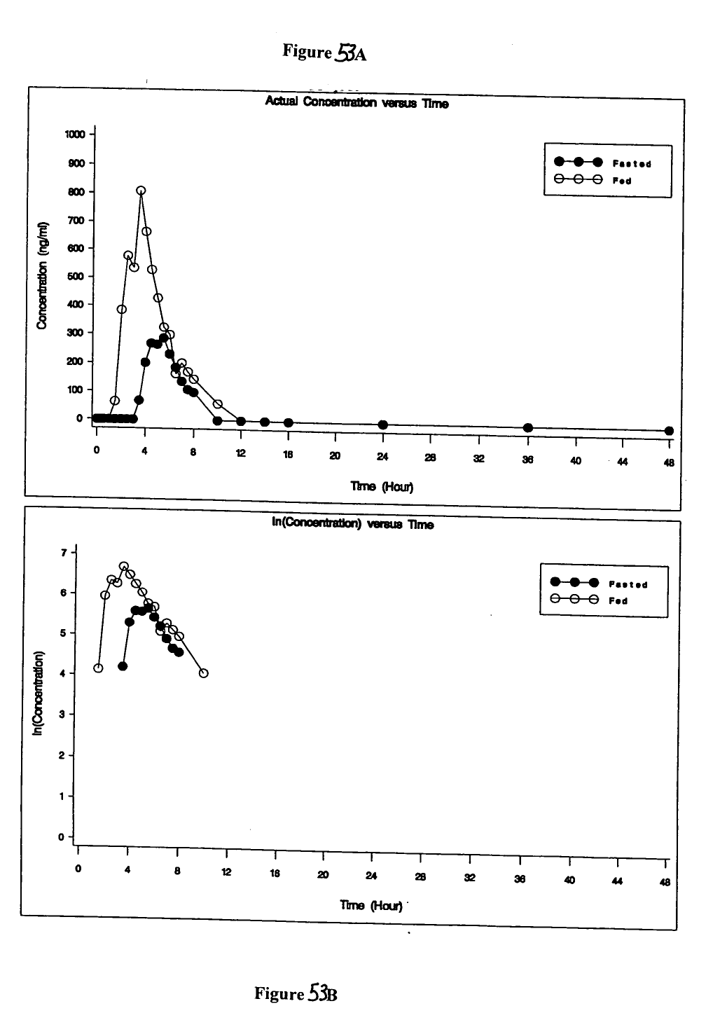

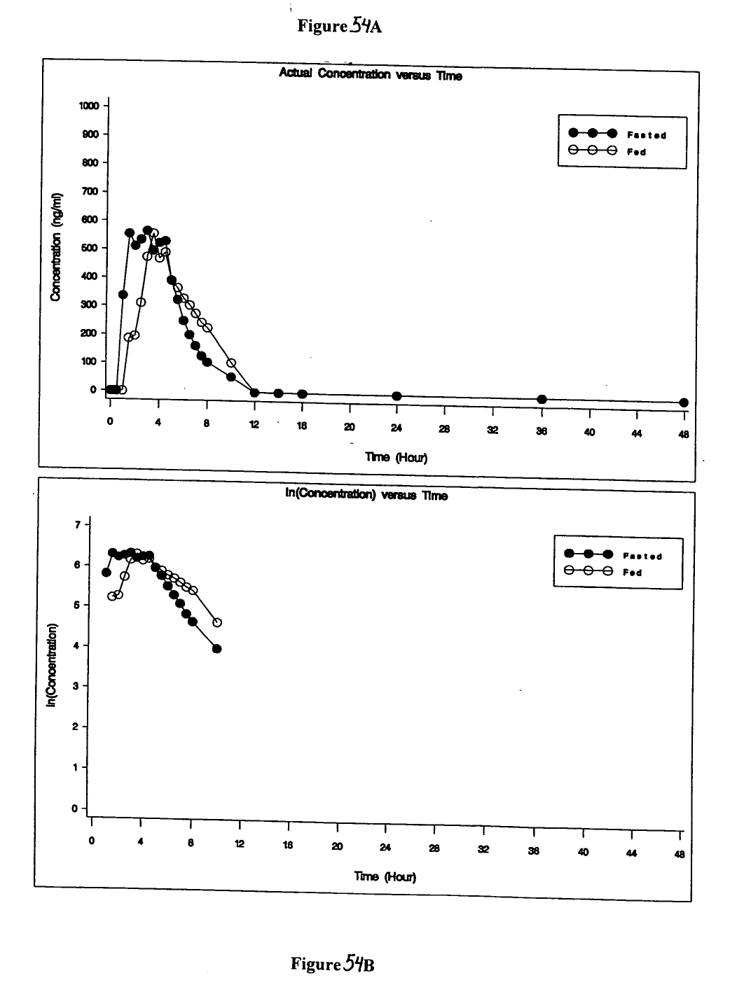

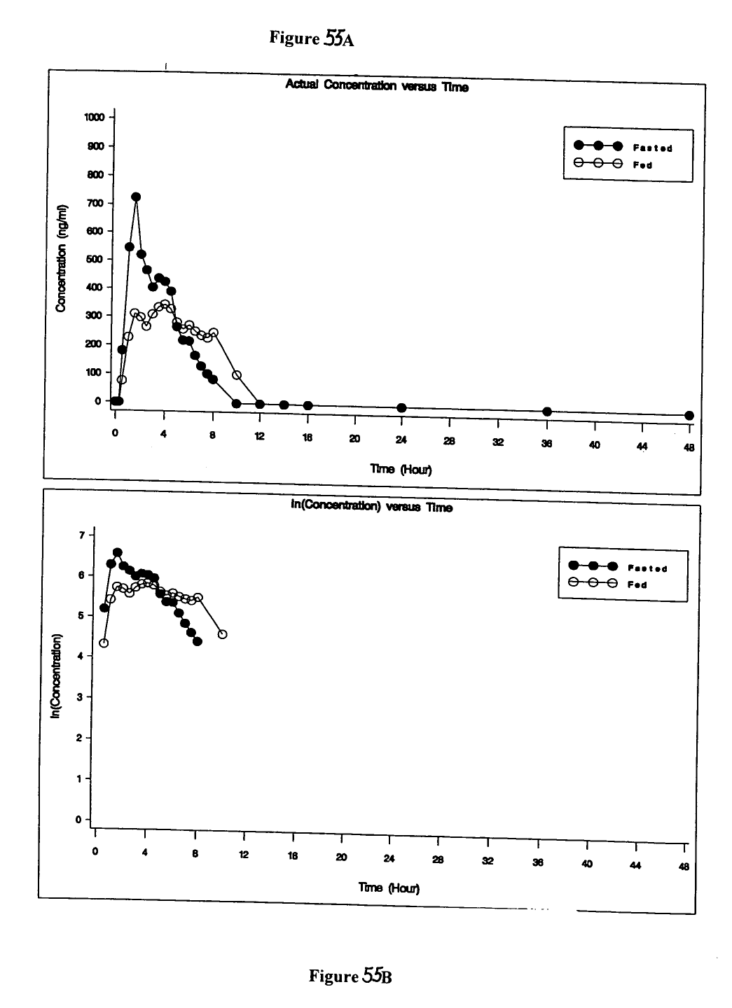

In embodiments, the invention is directed to an extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in any one of FIGS. 10-55.

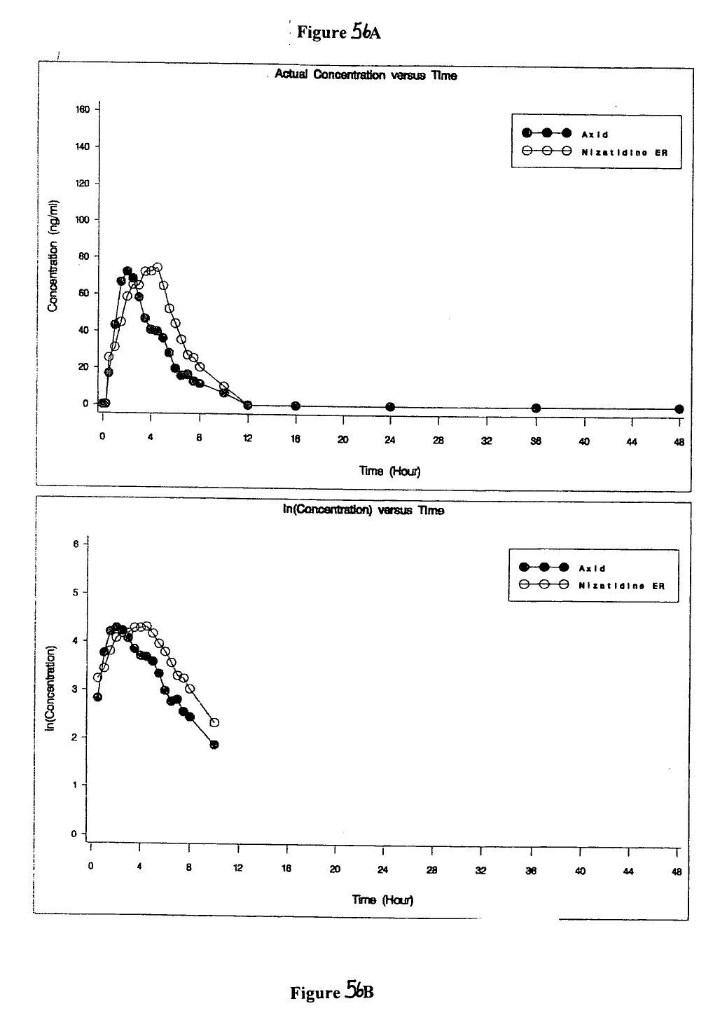

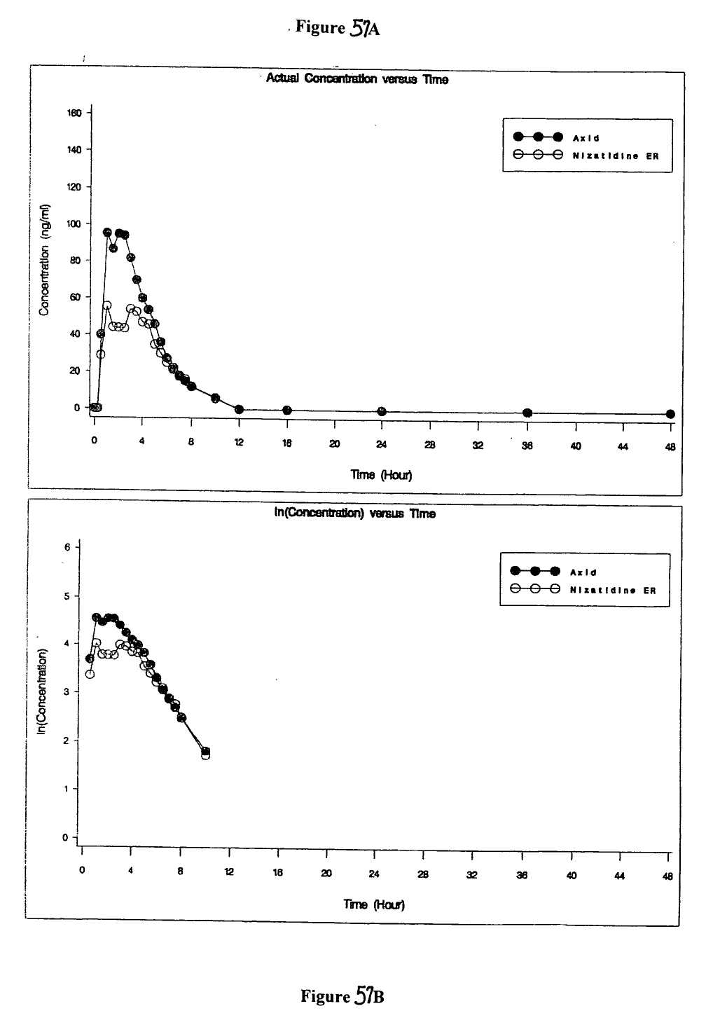

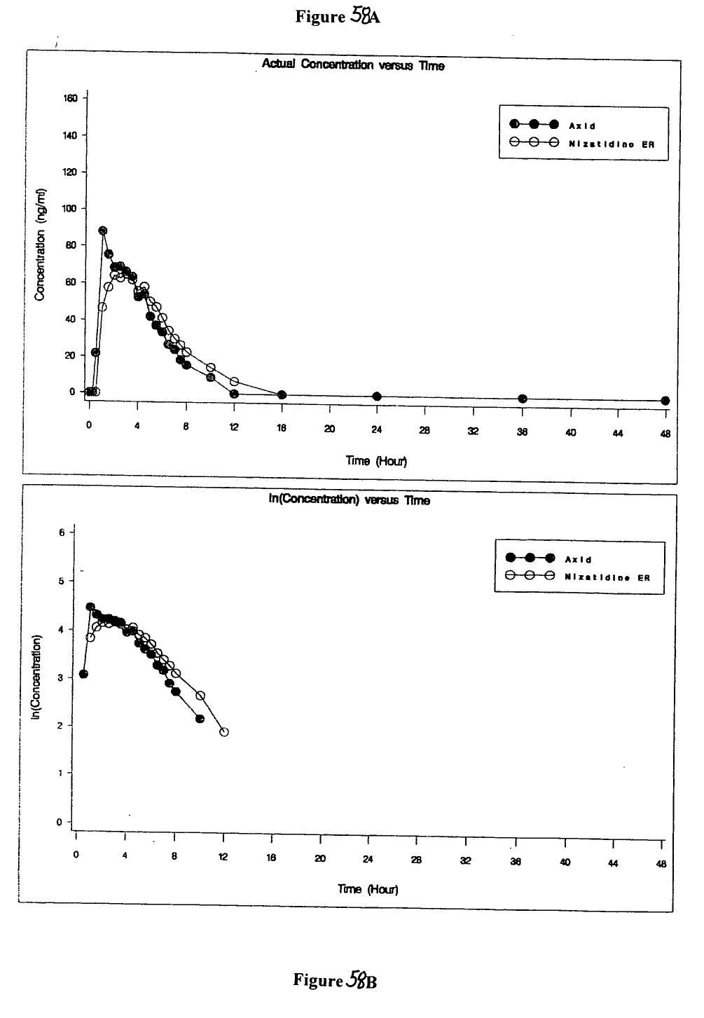

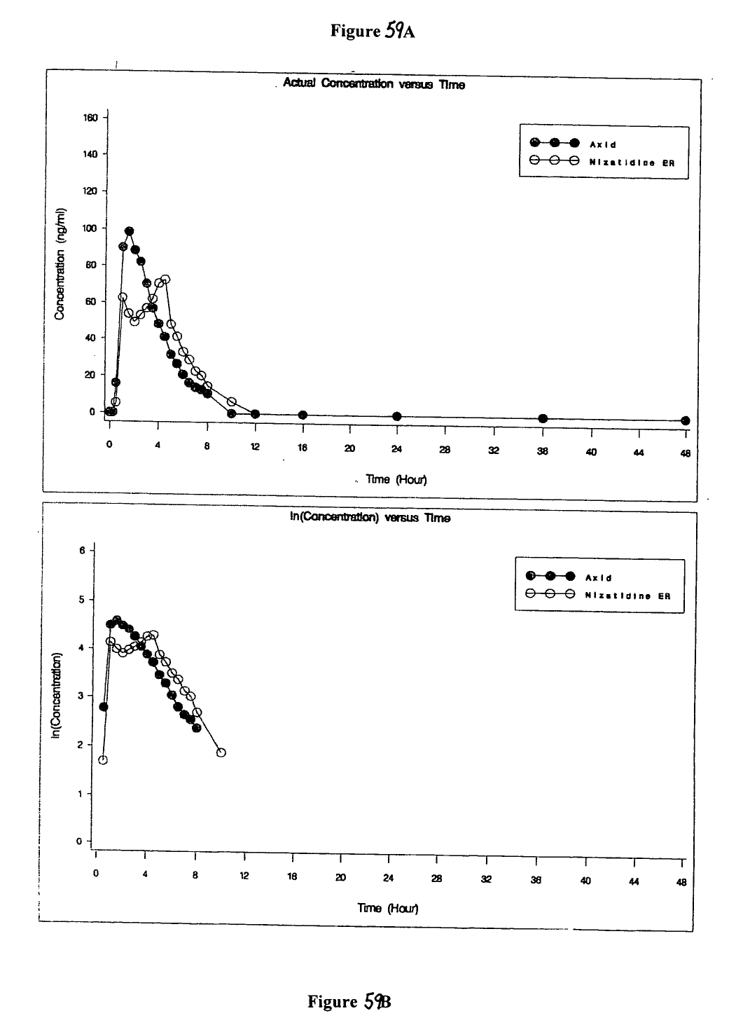

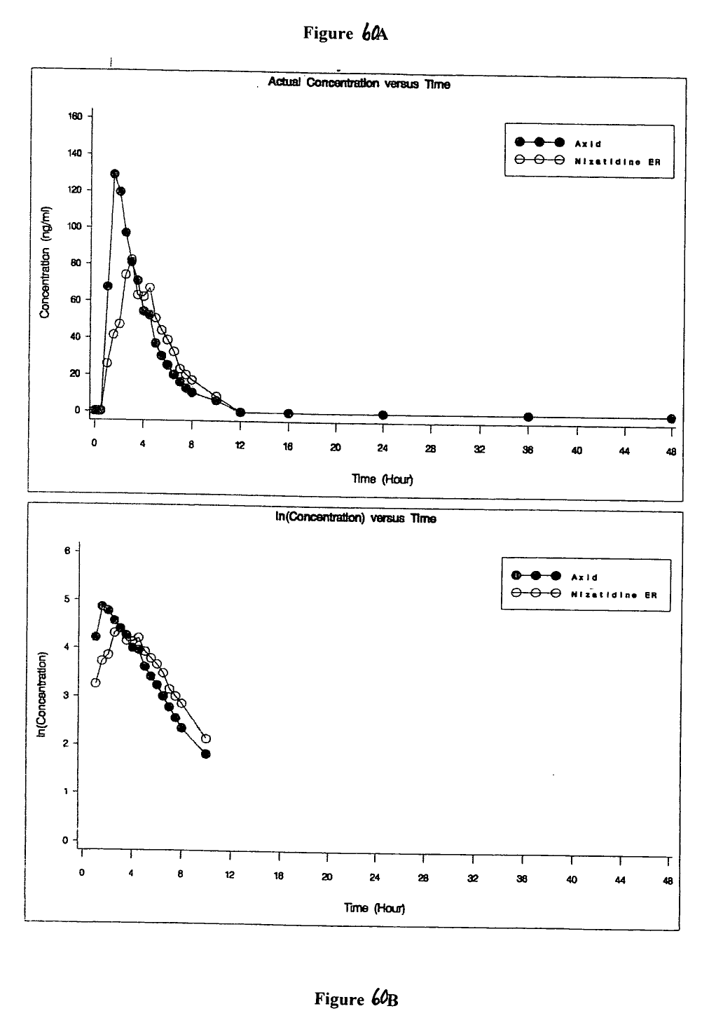

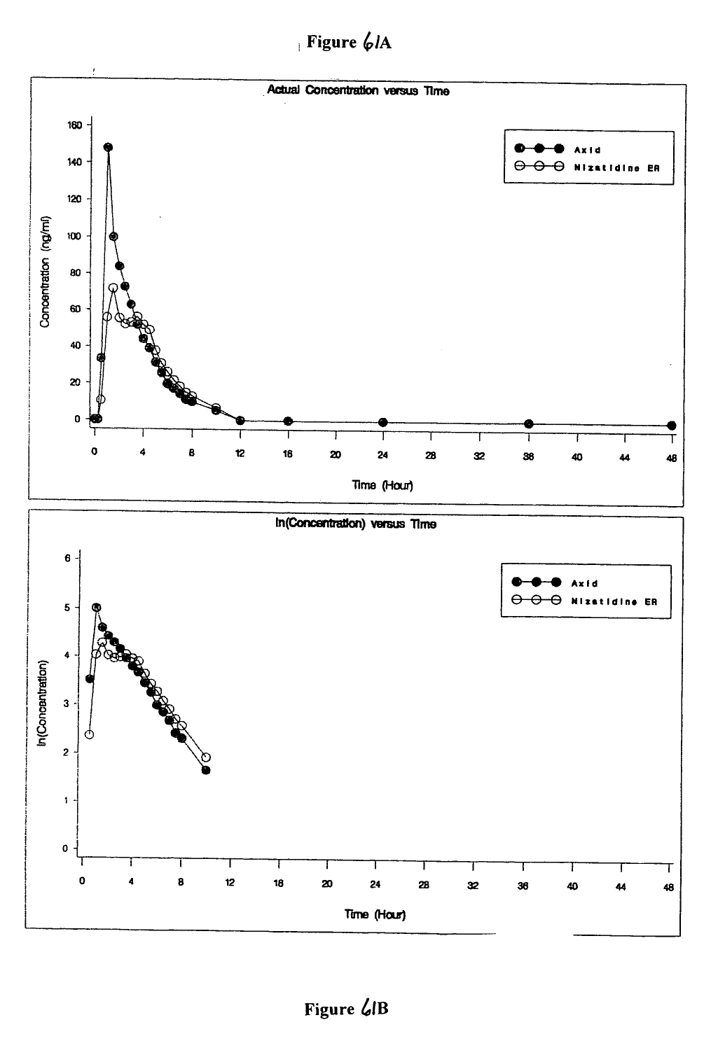

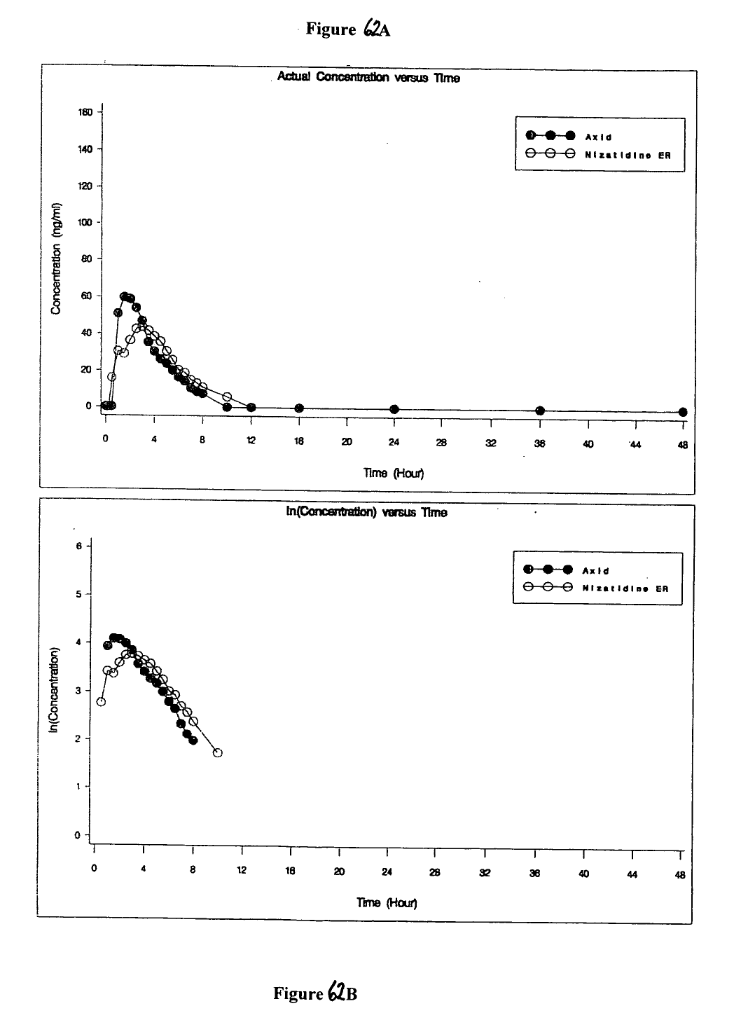

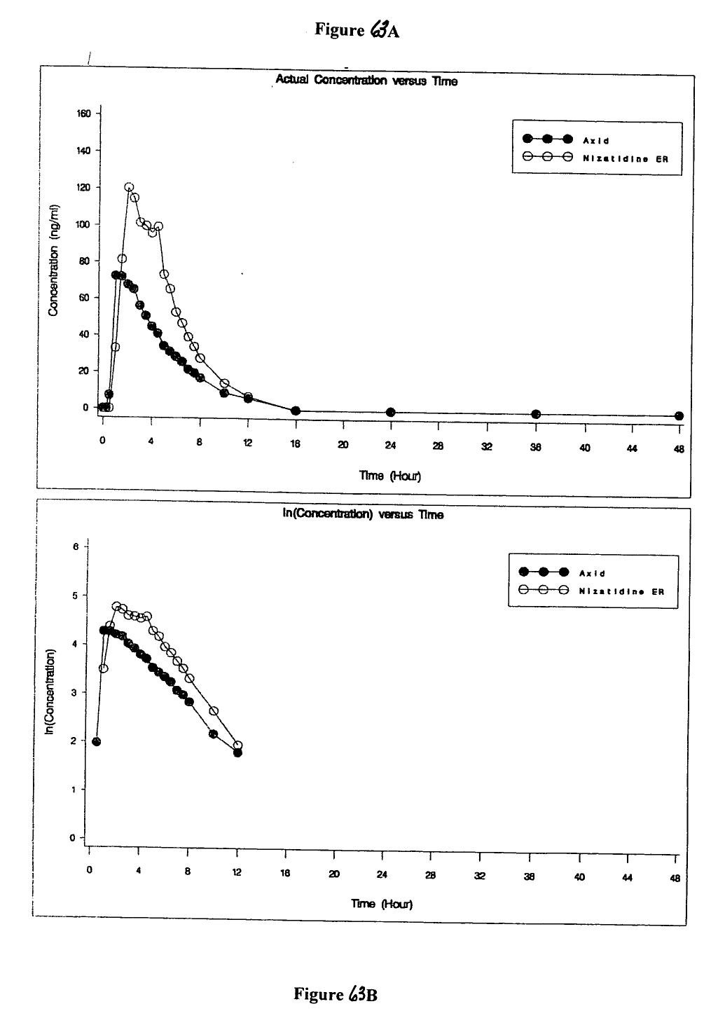

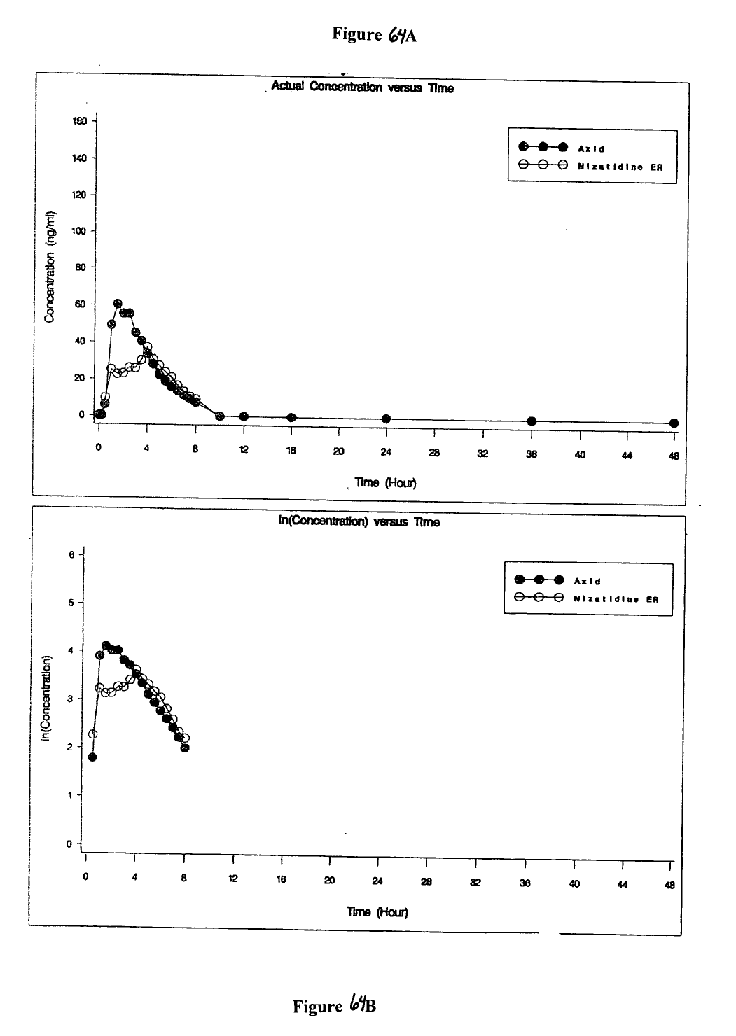

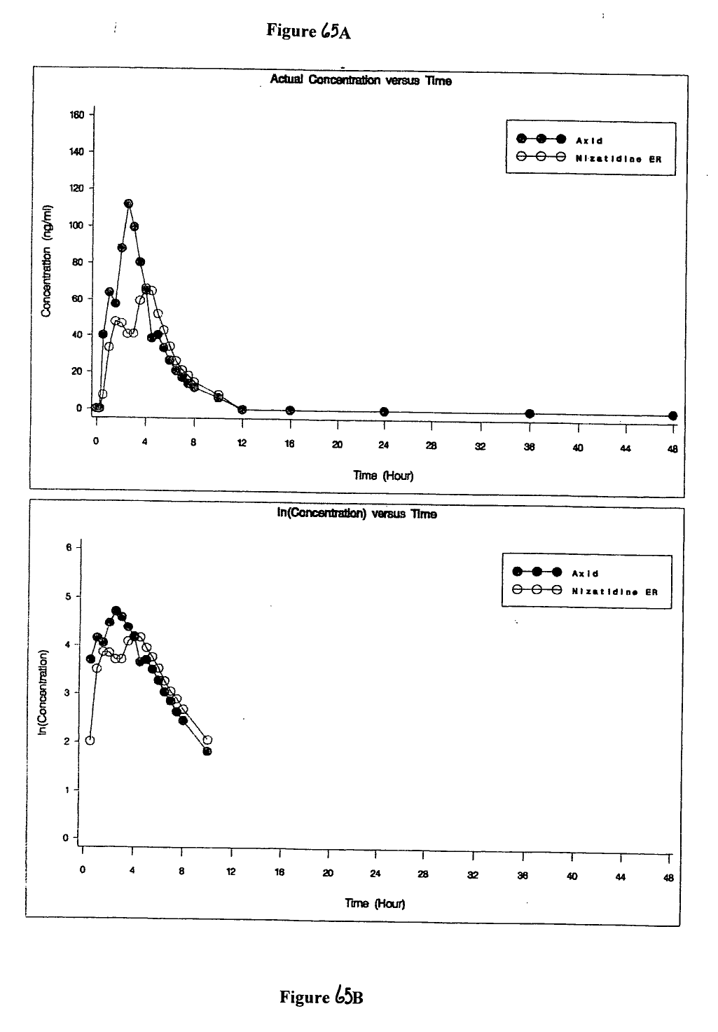

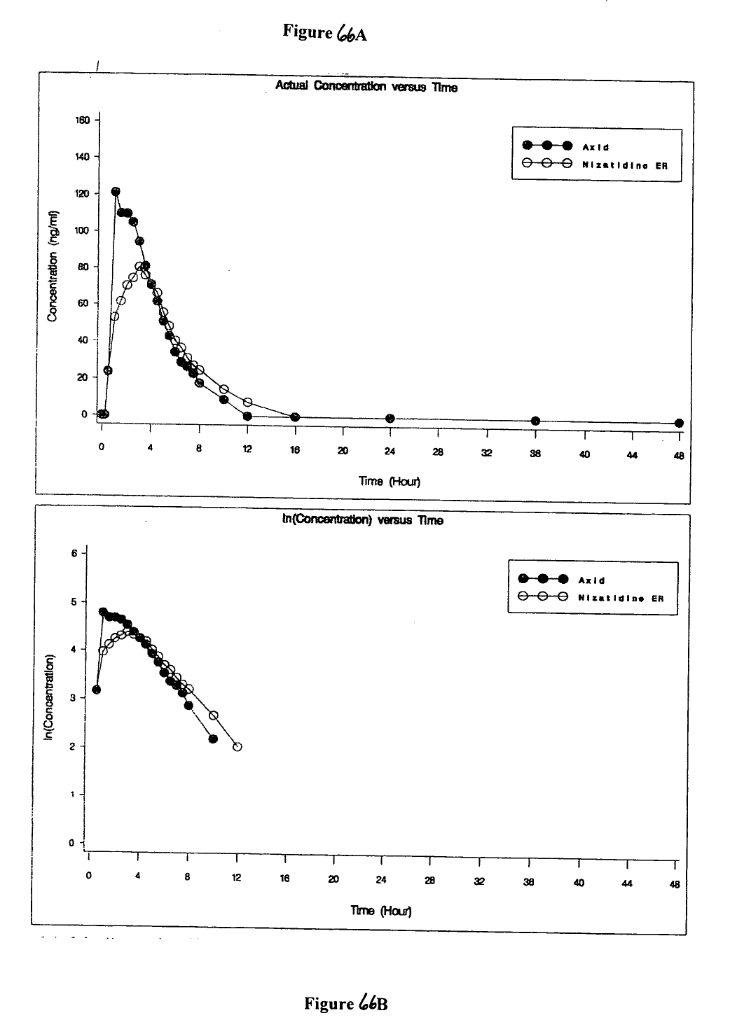

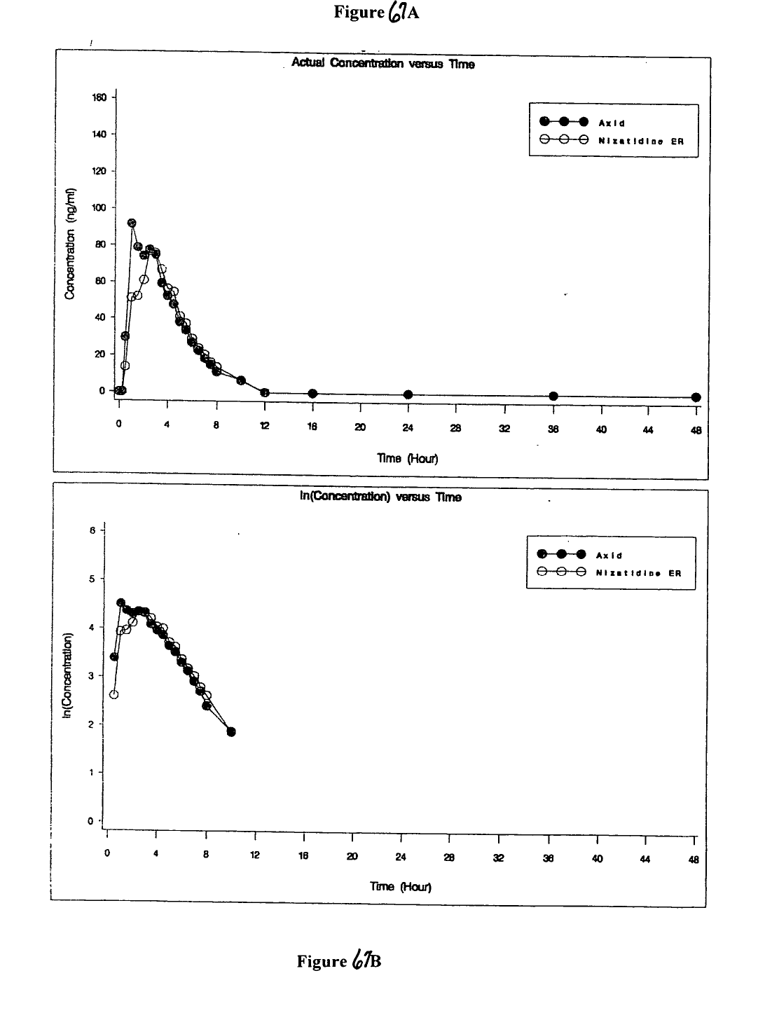

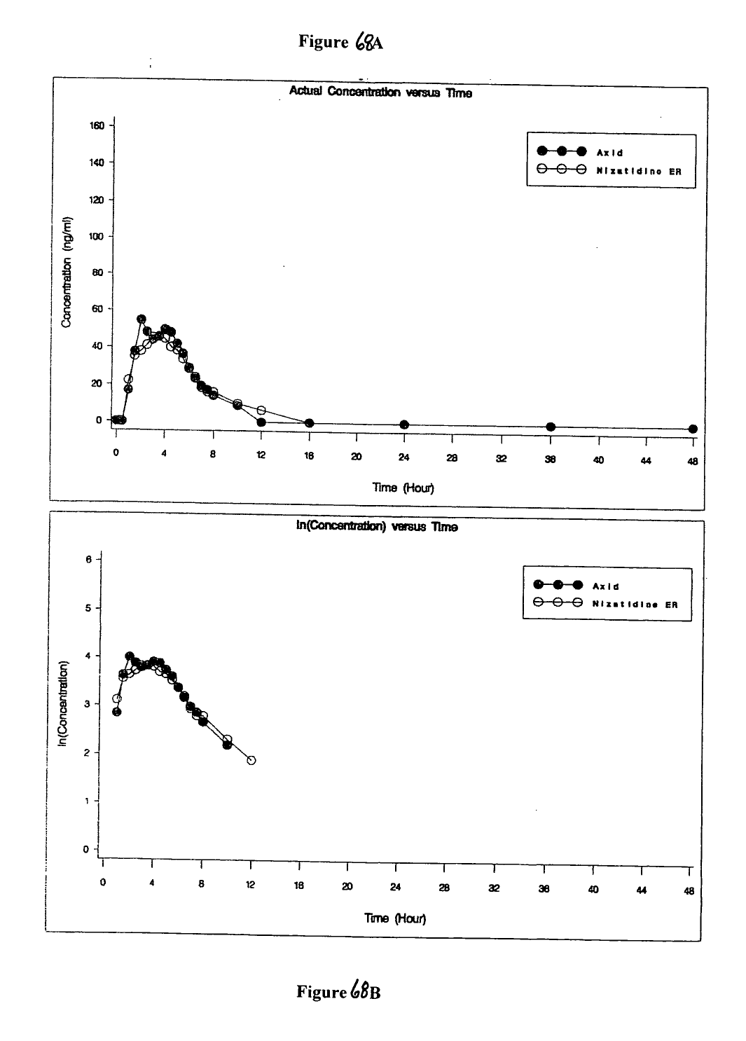

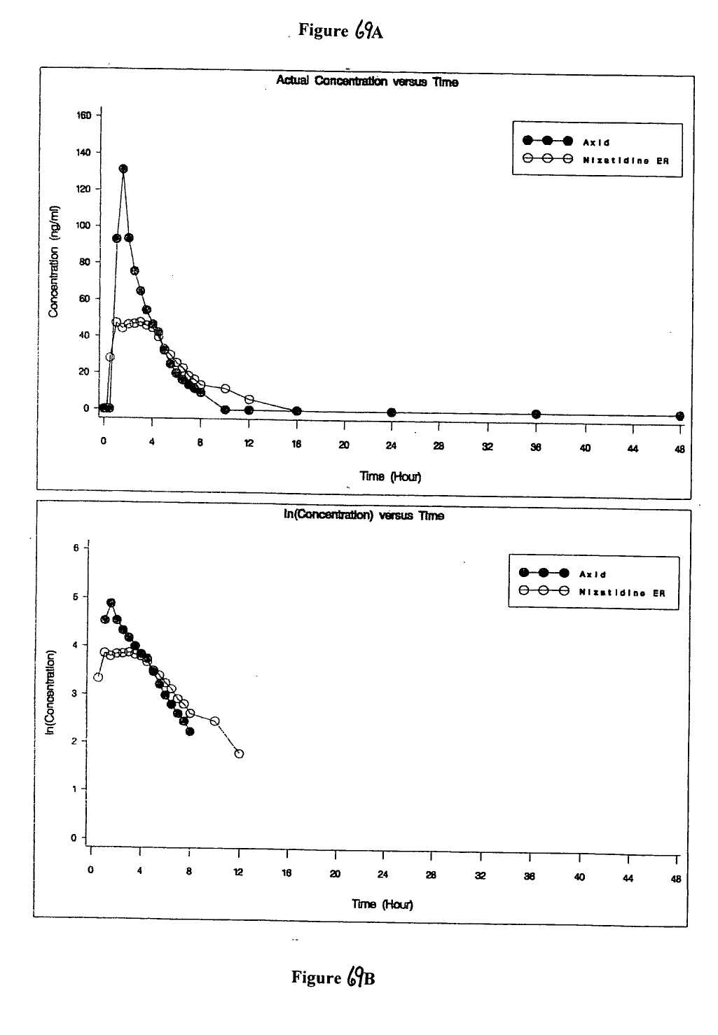

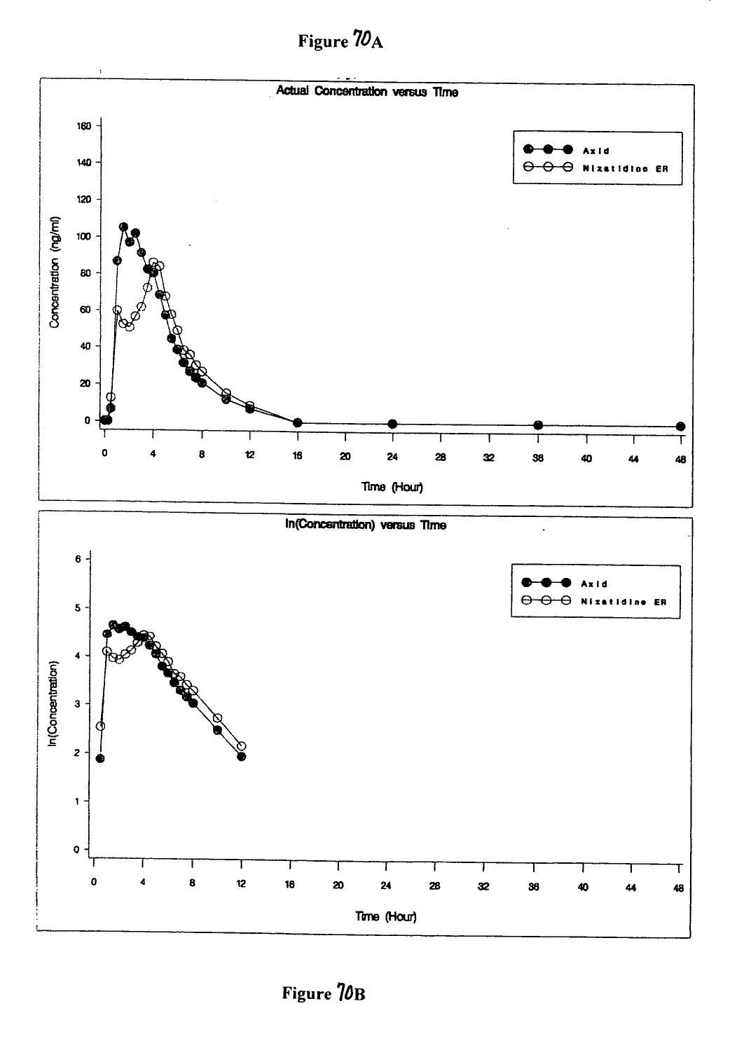

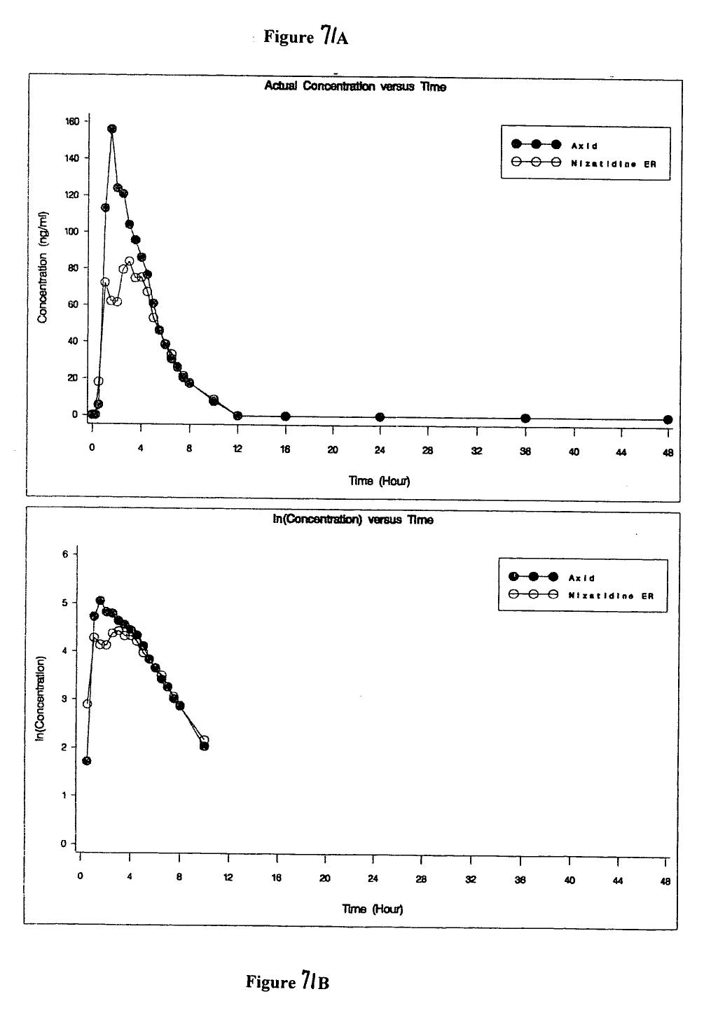

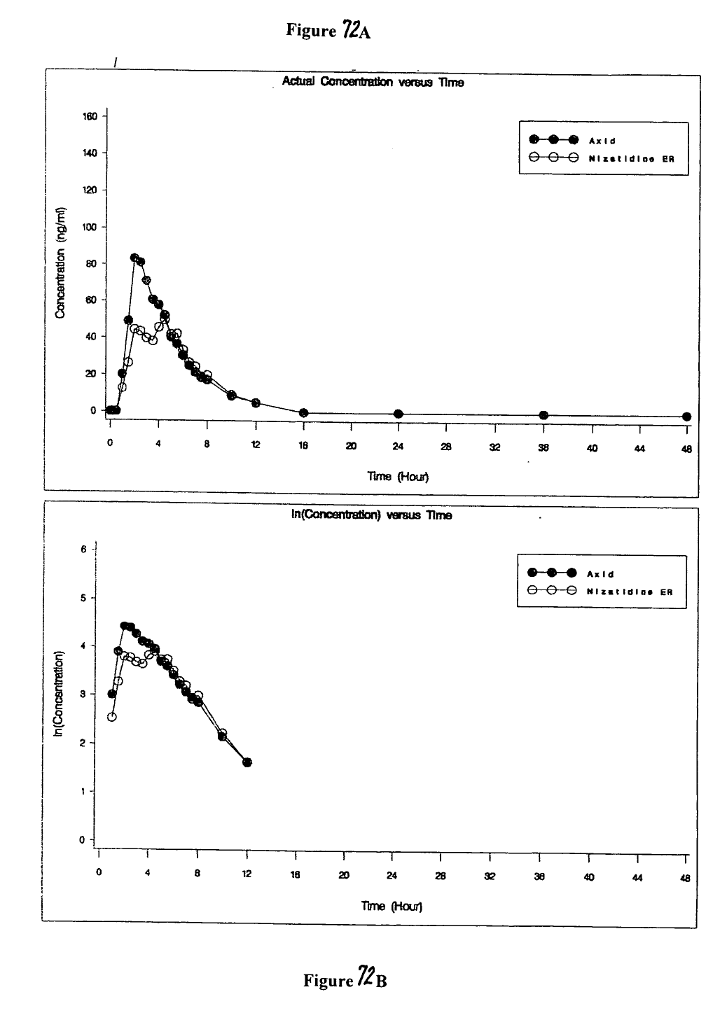

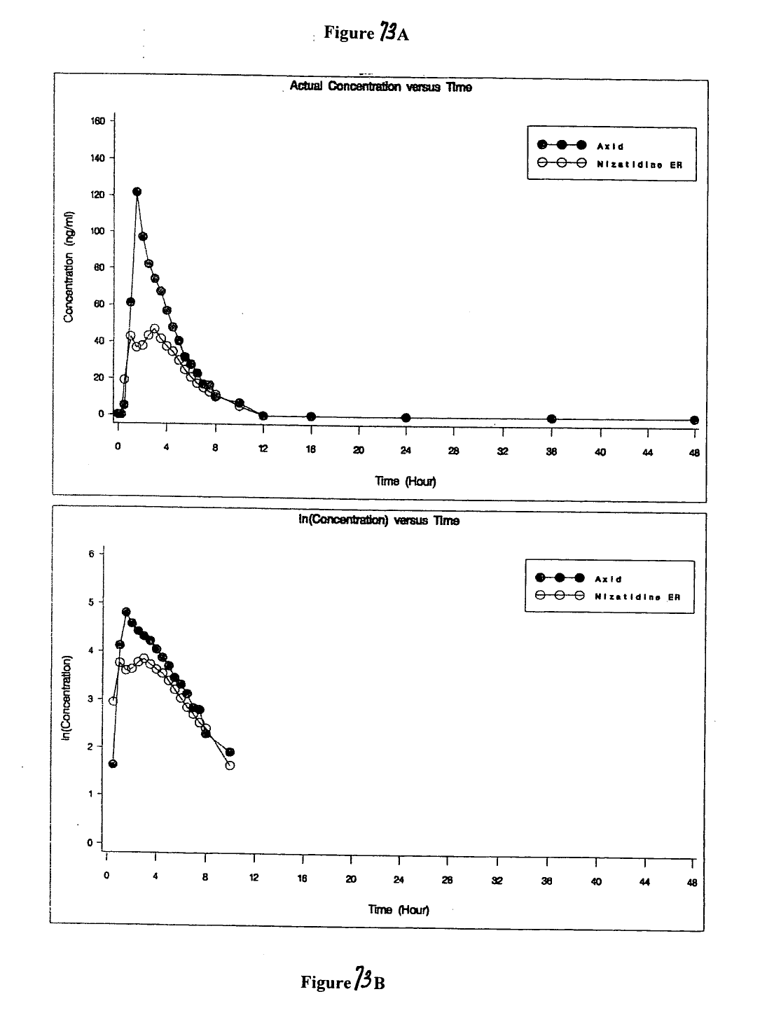

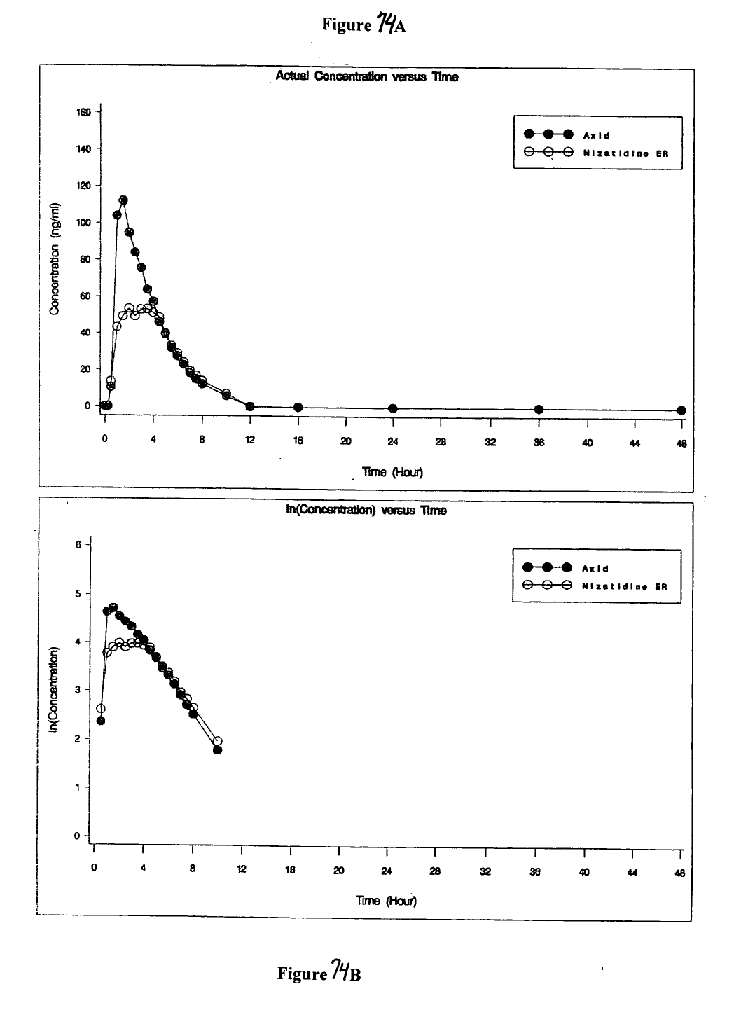

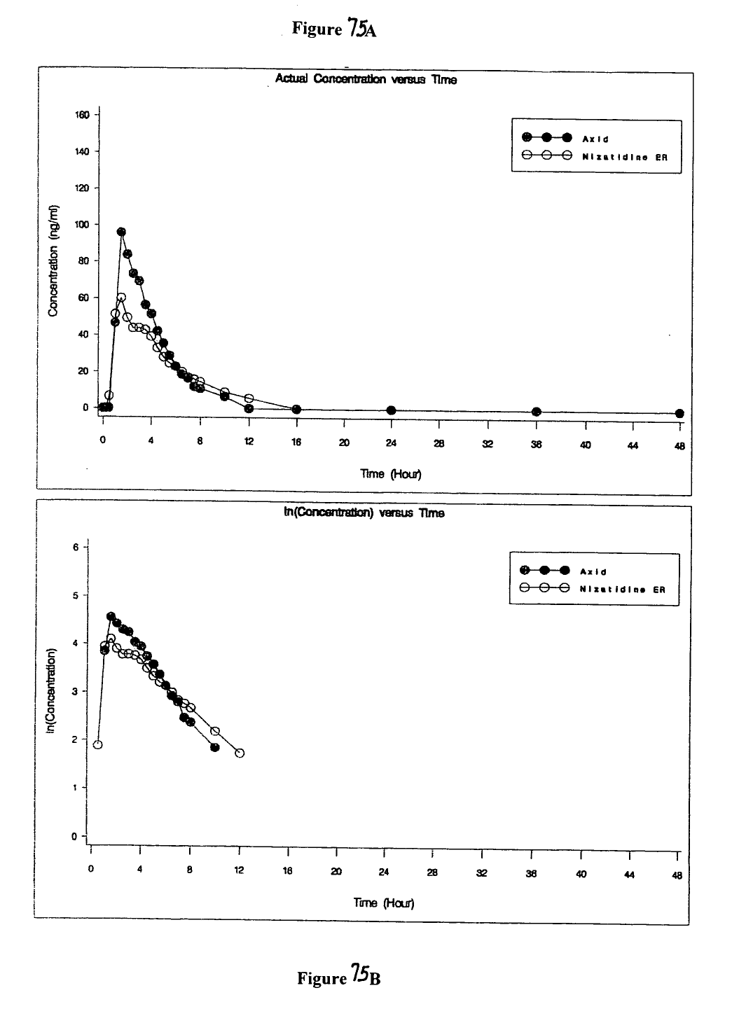

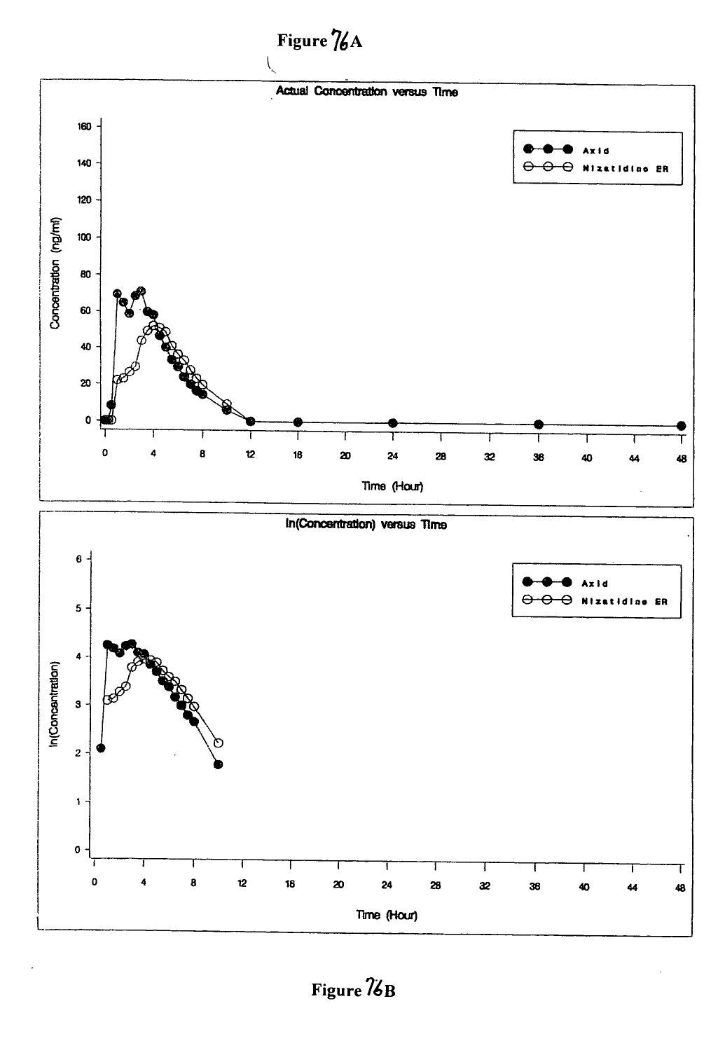

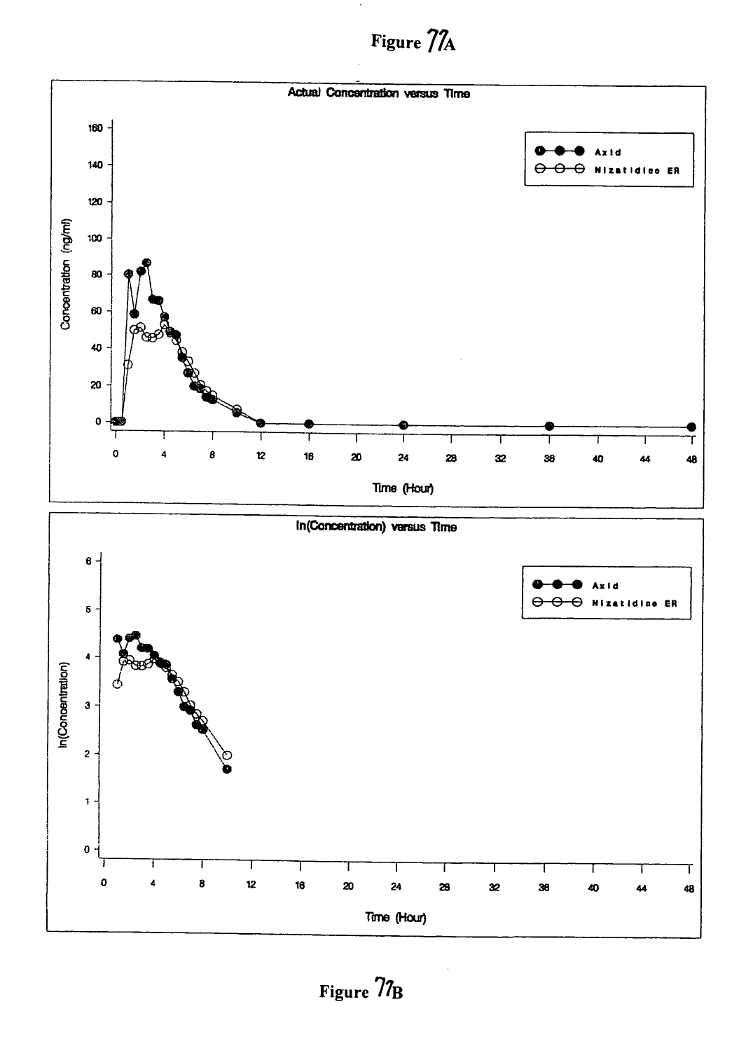

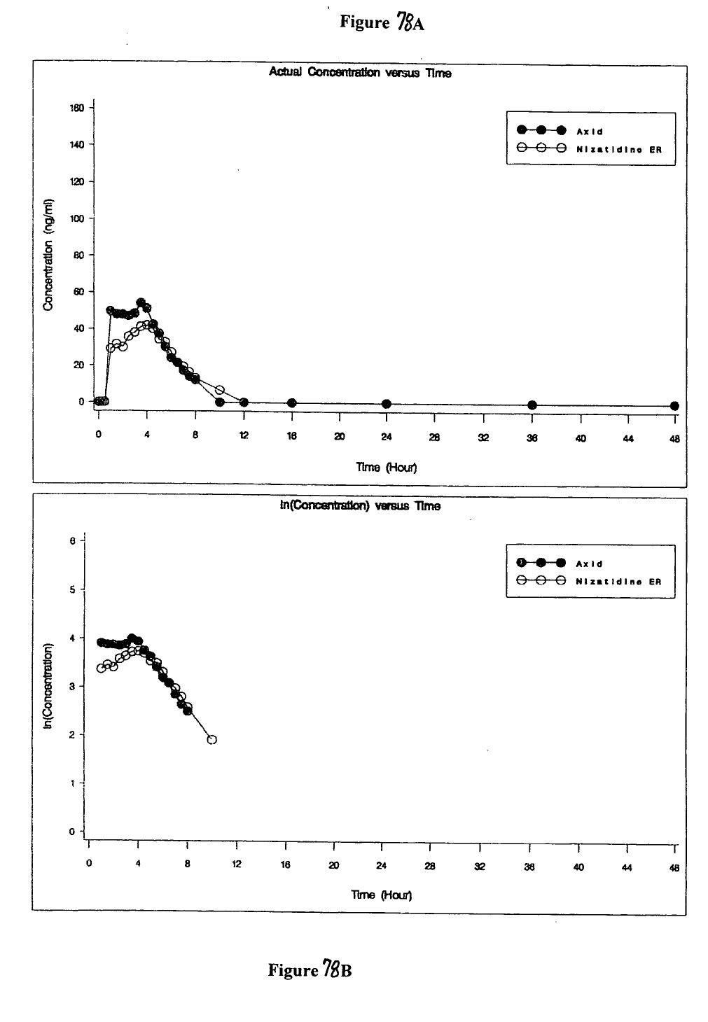

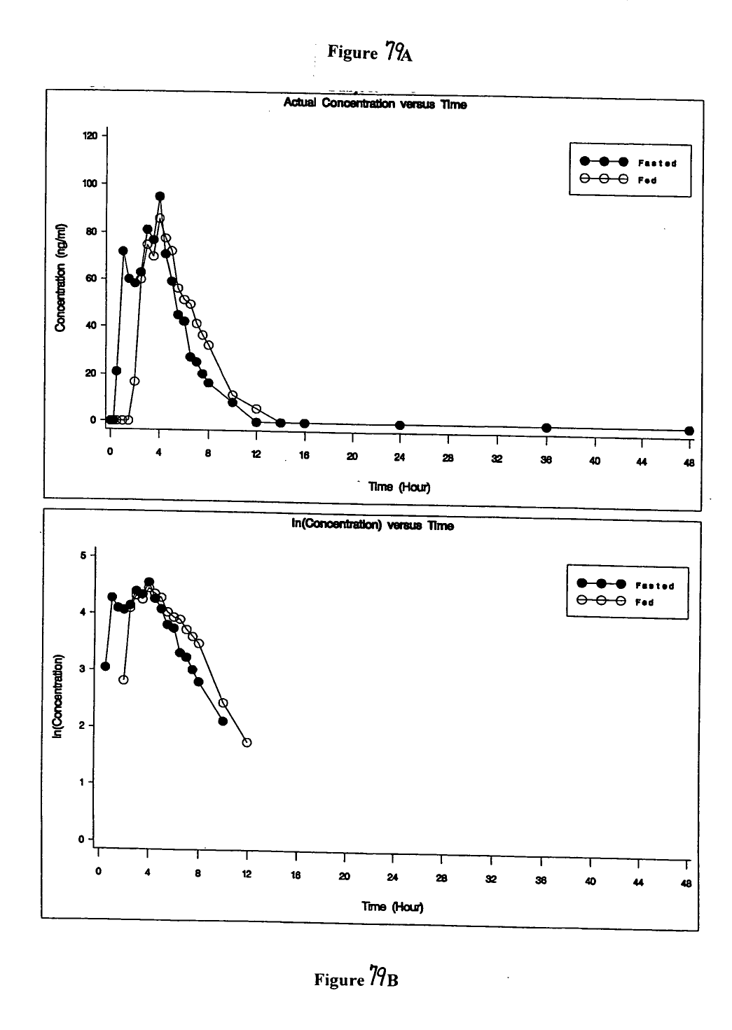

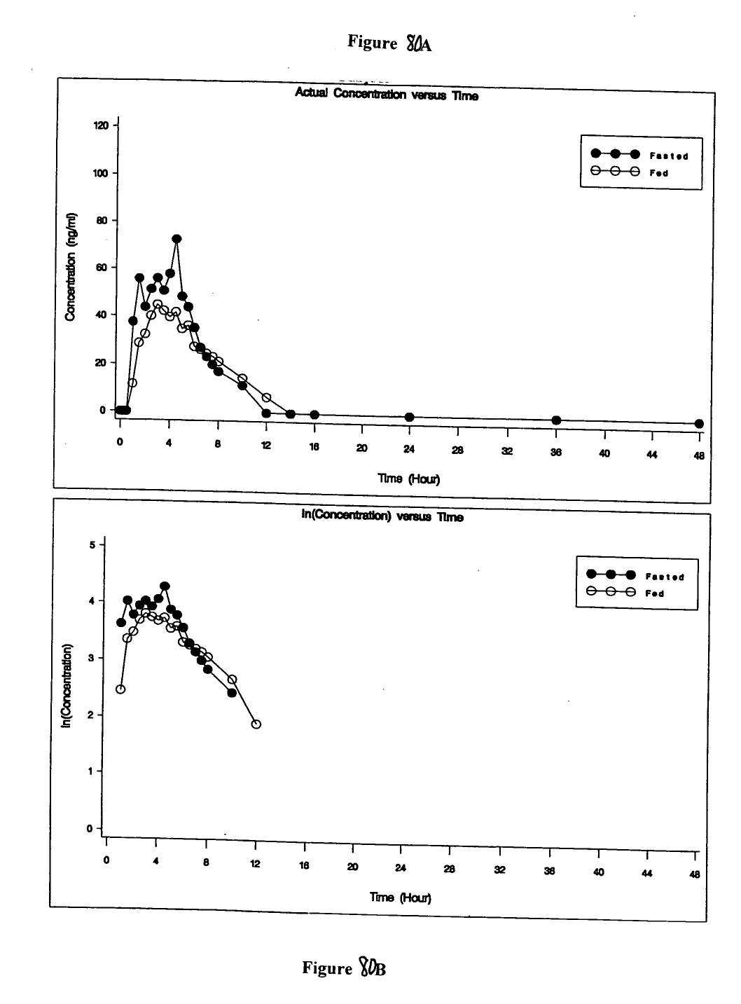

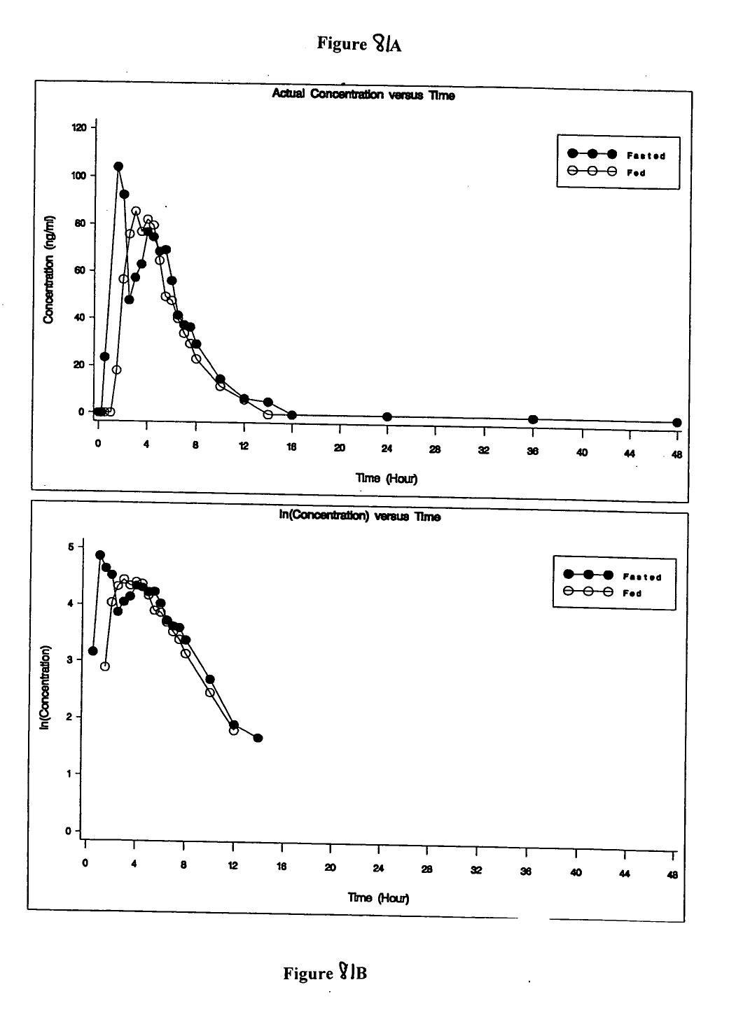

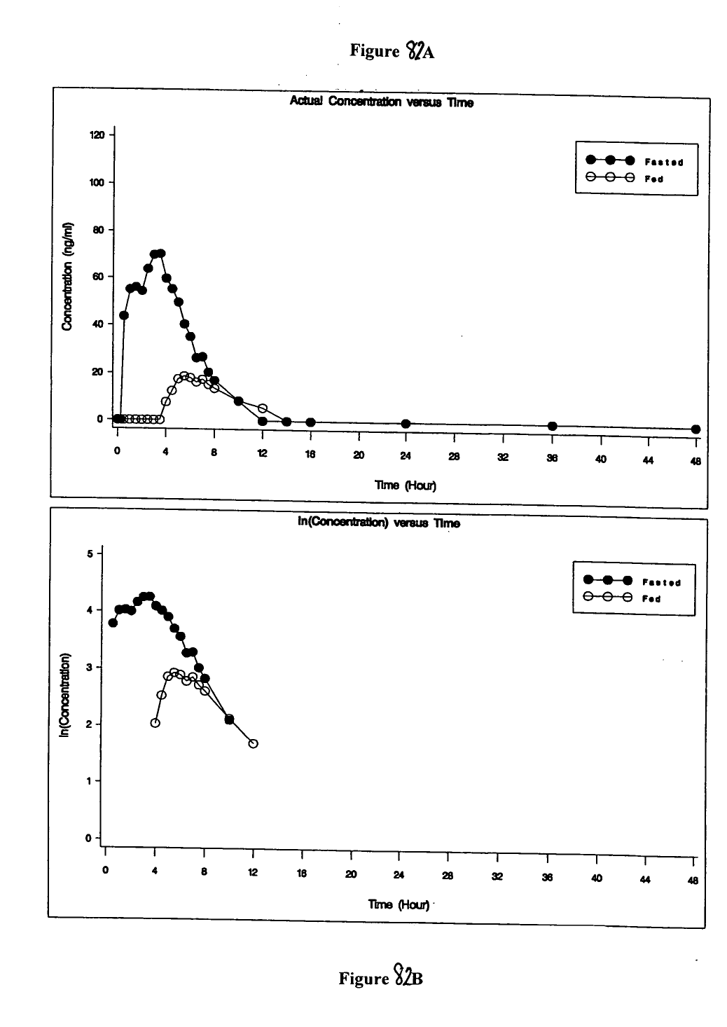

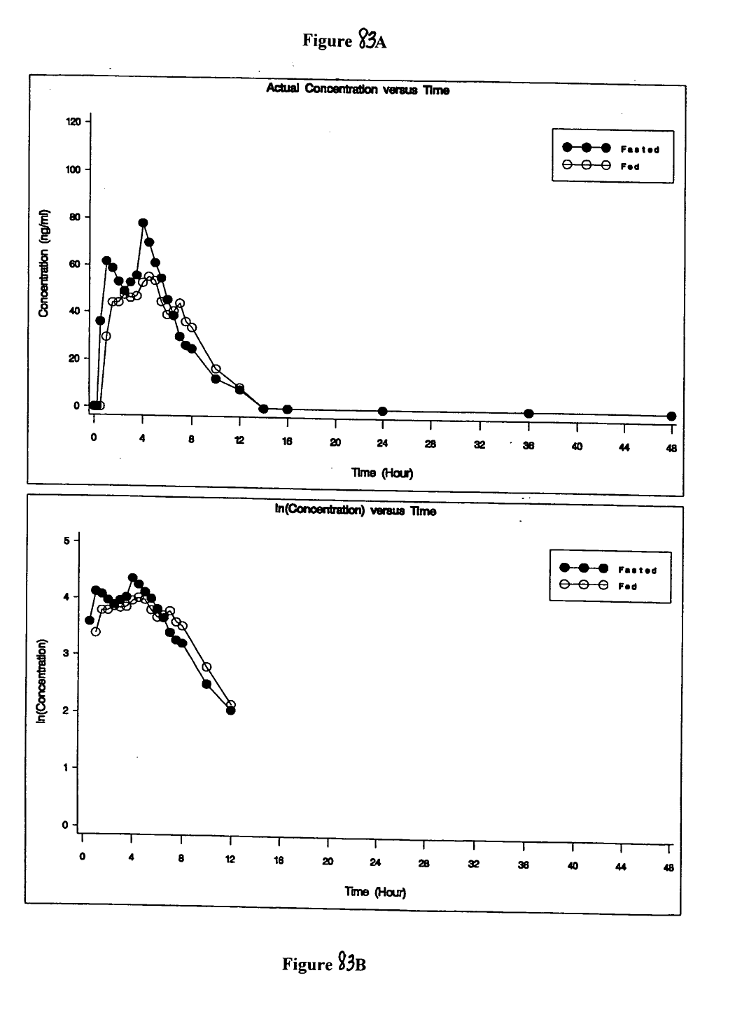

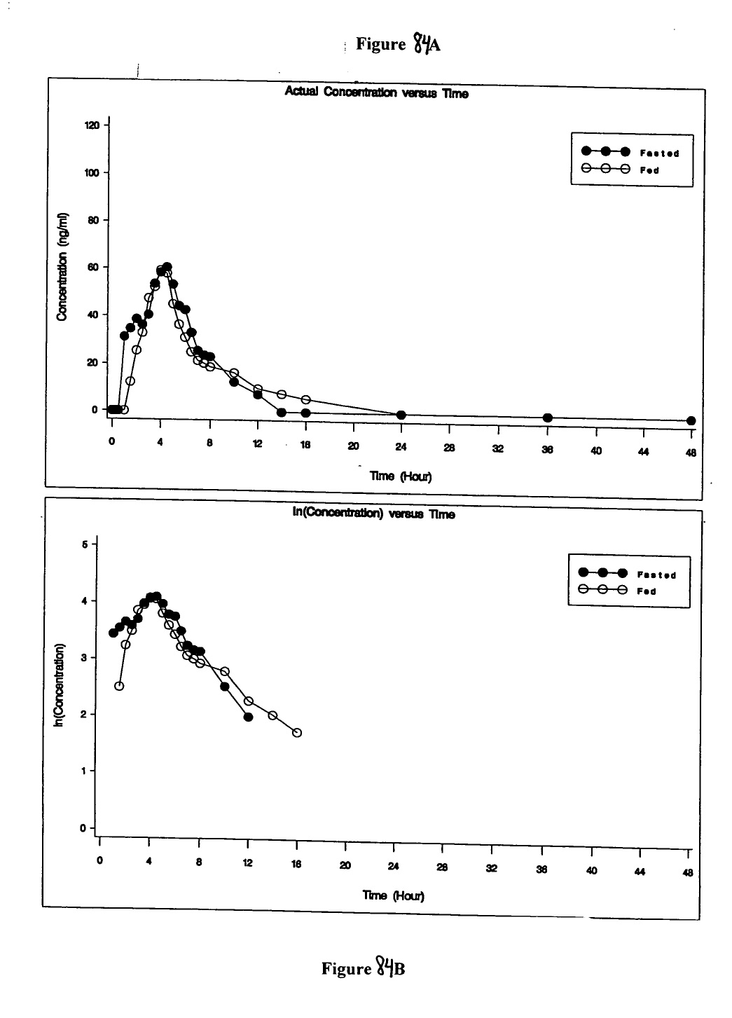

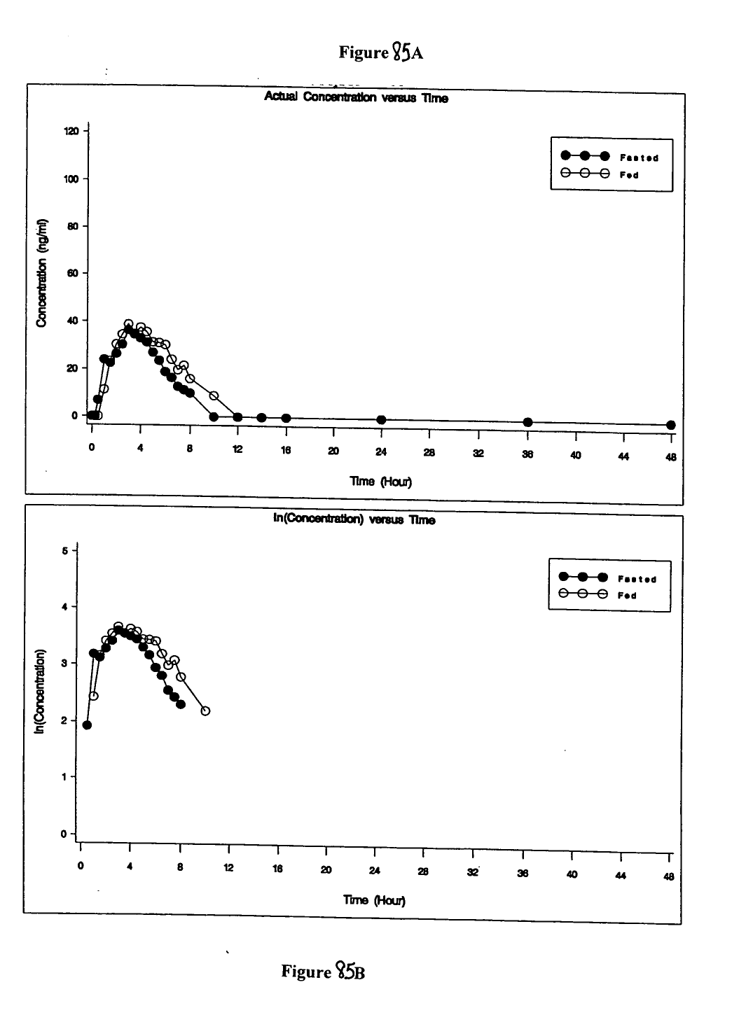

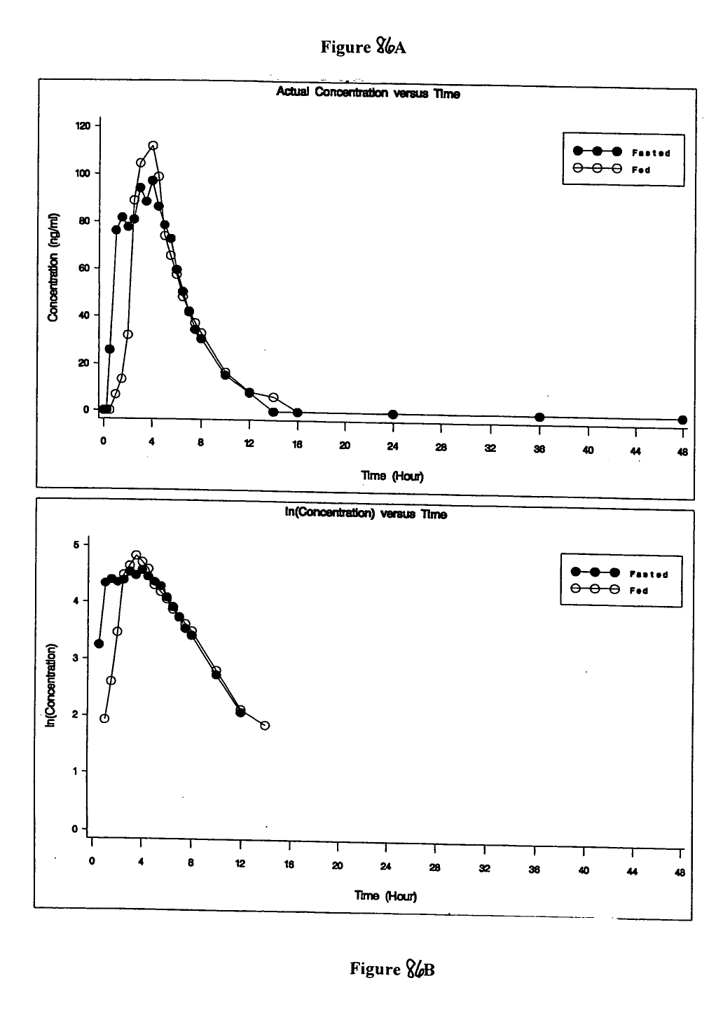

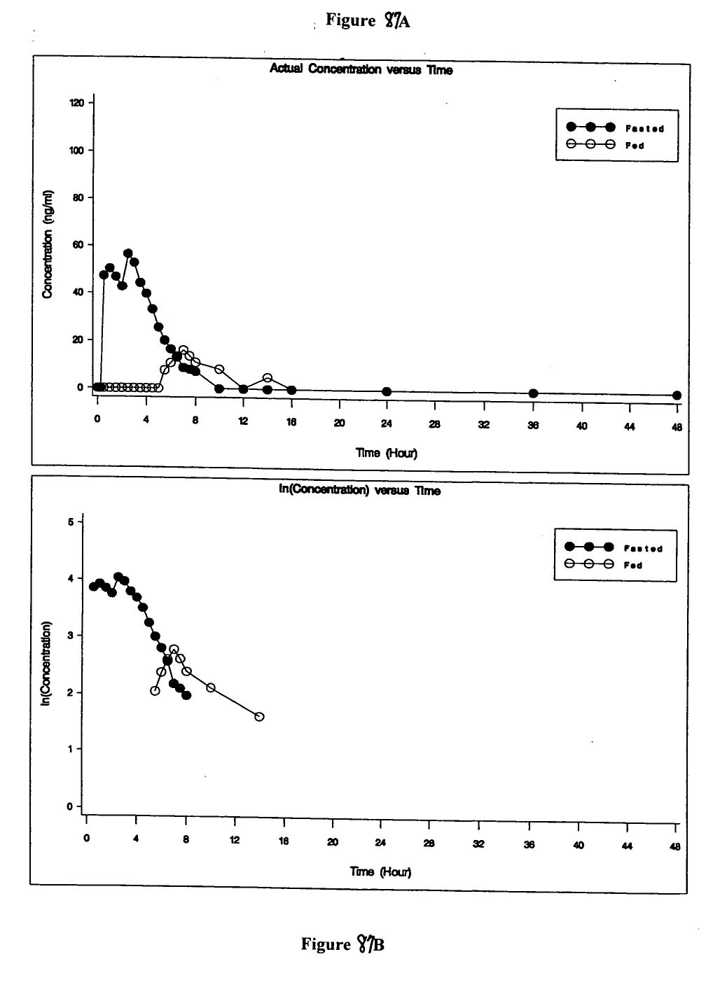

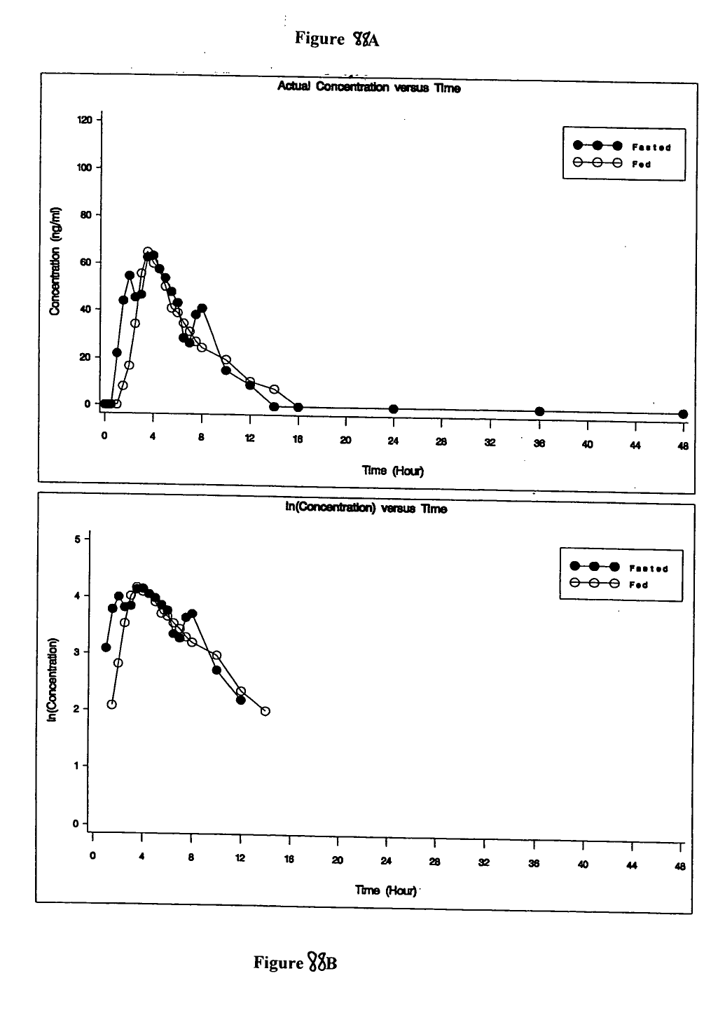

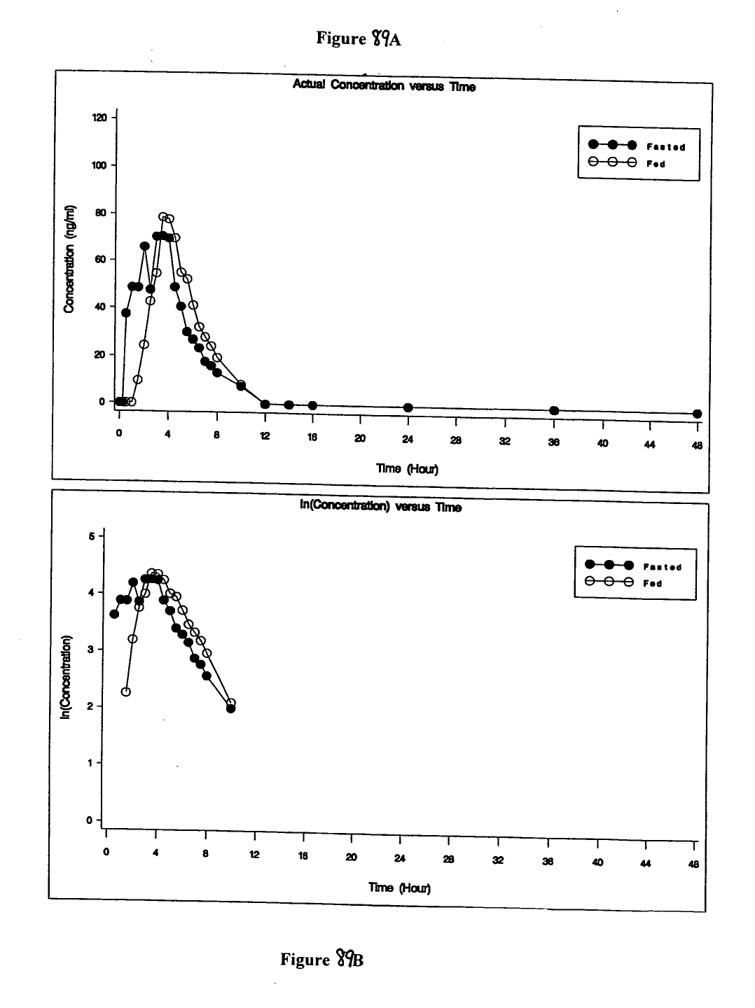

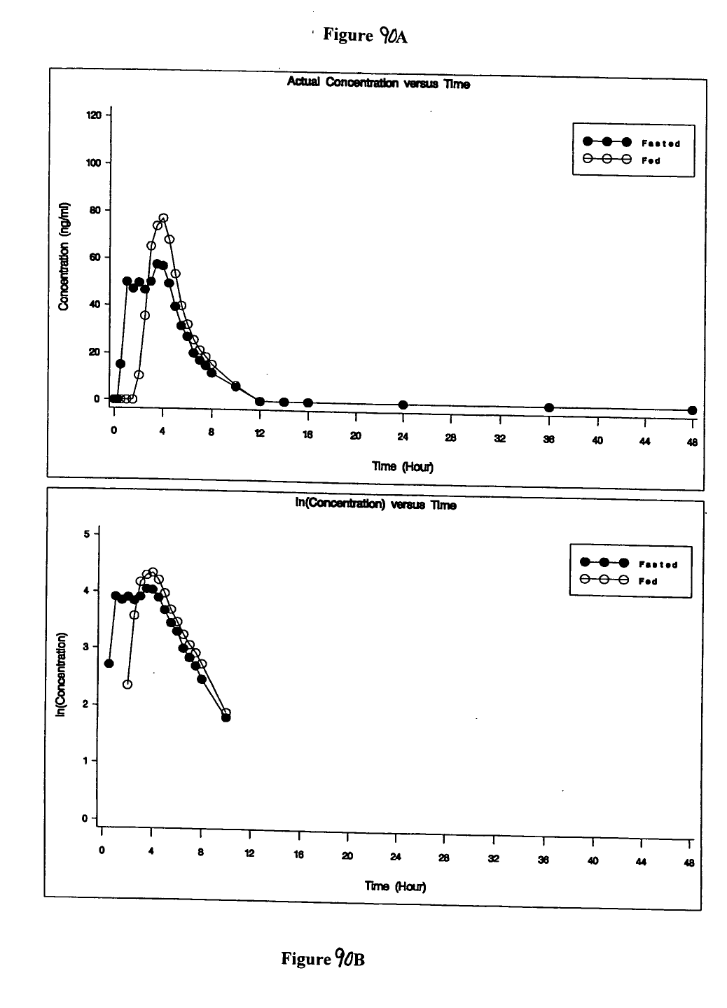

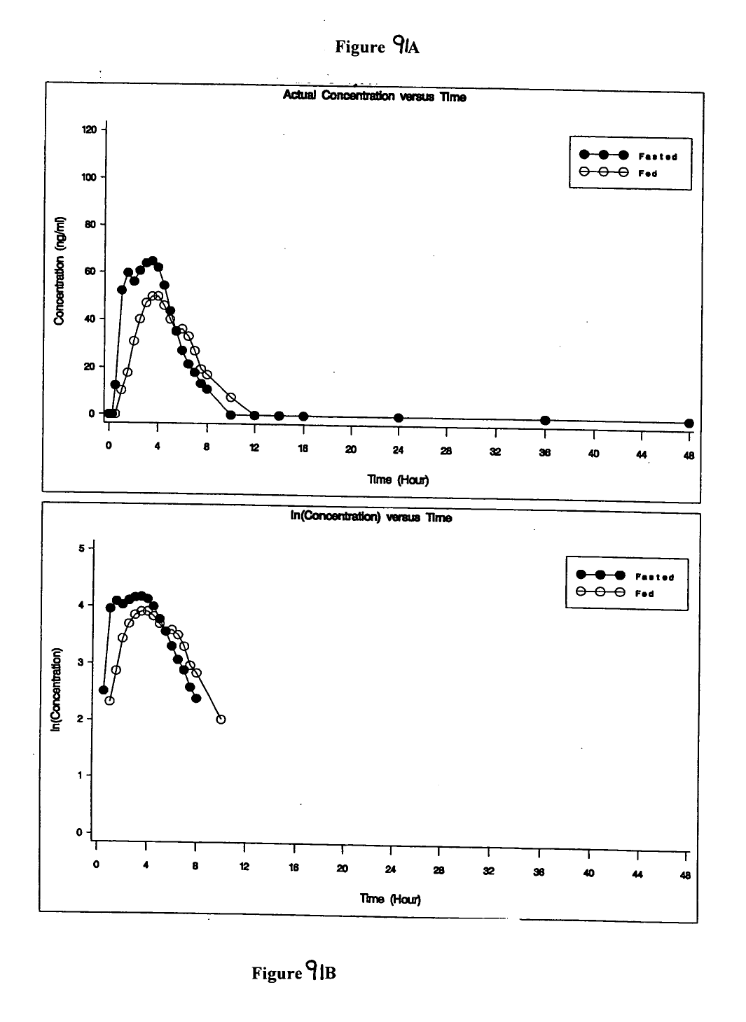

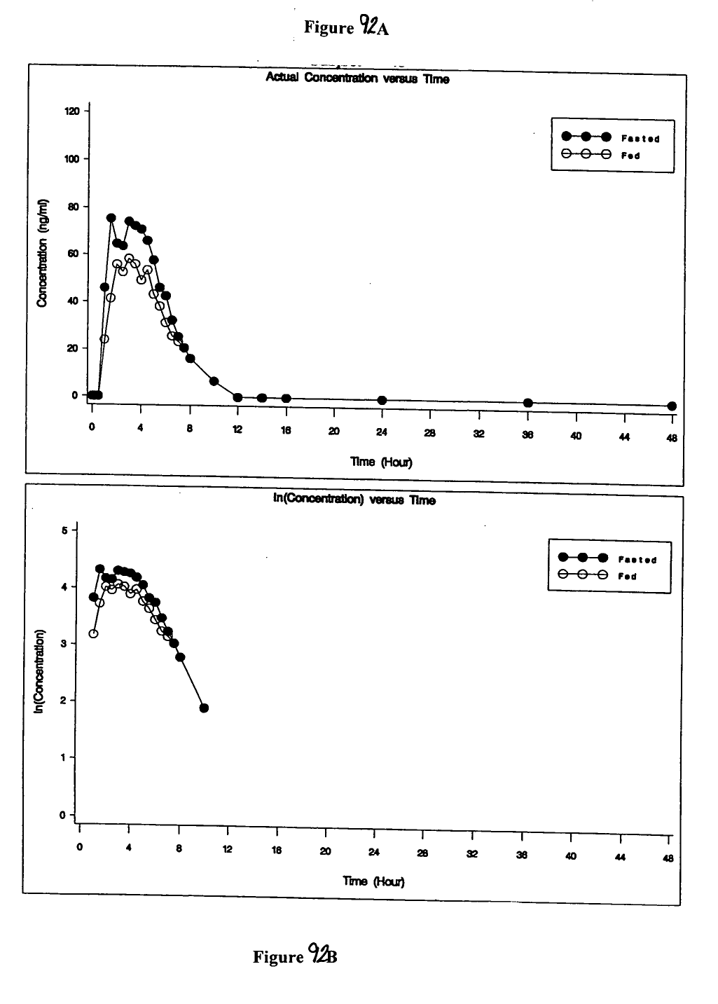

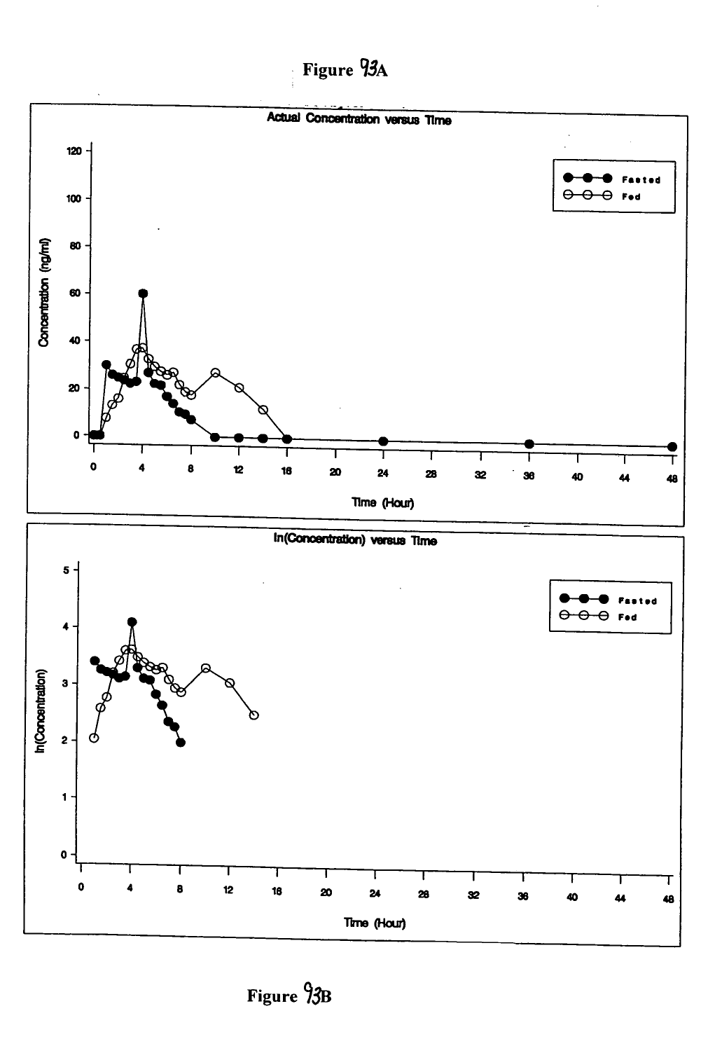

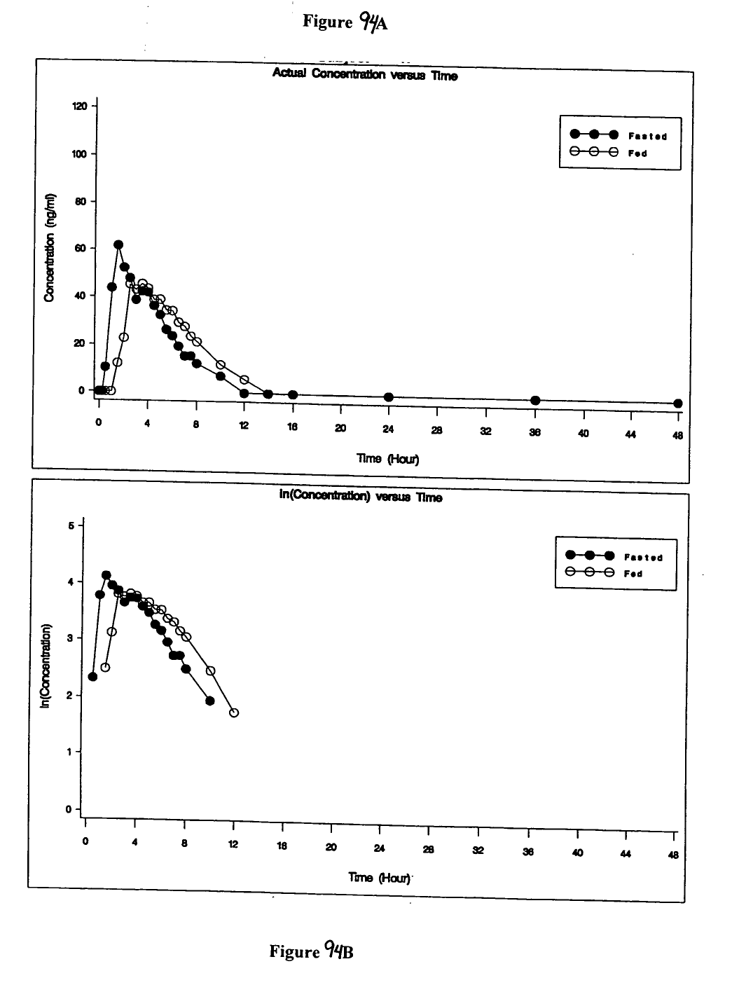

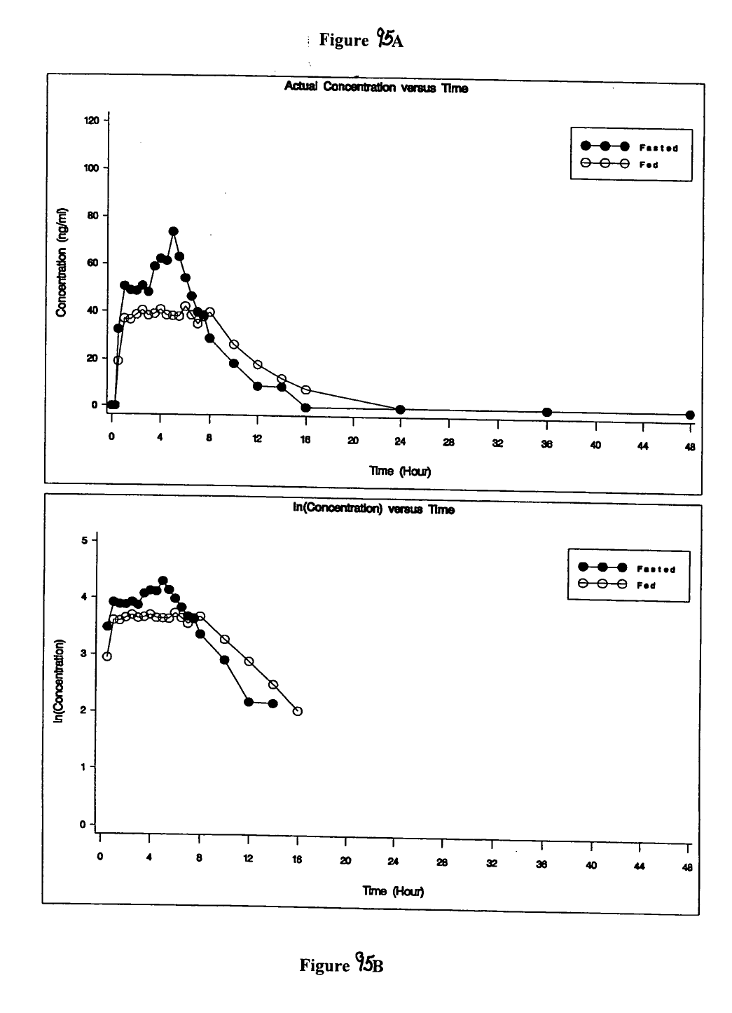

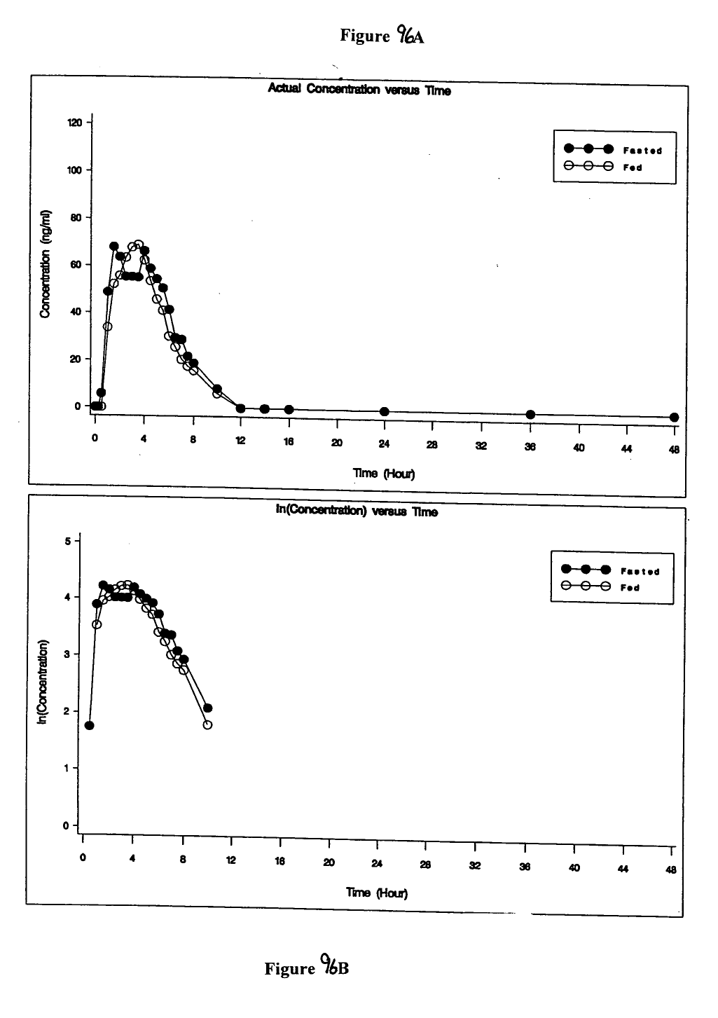

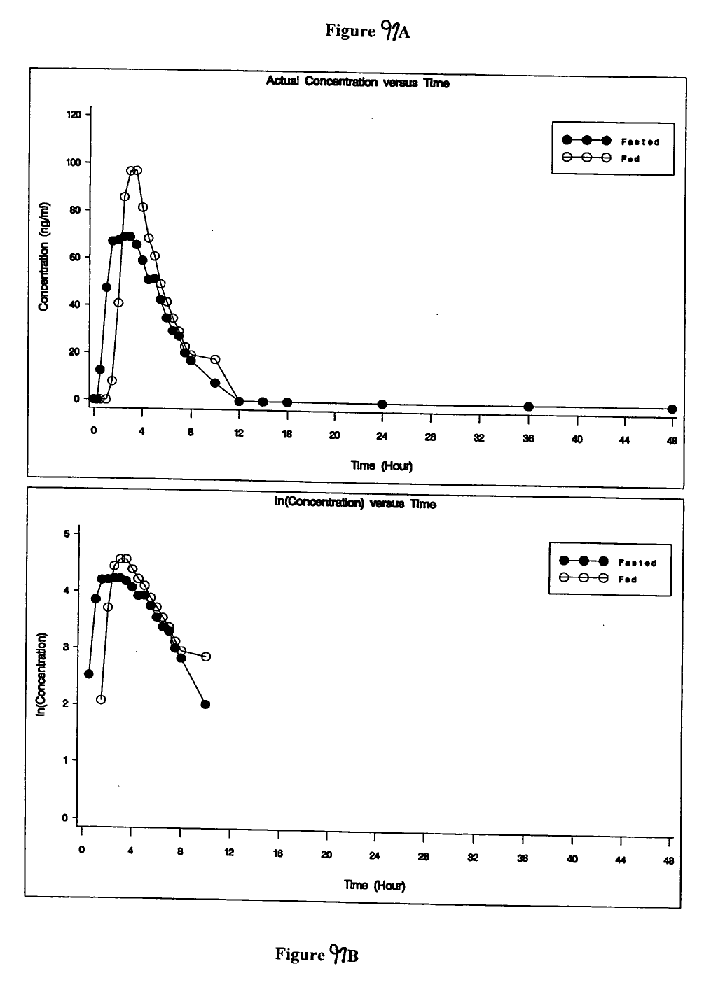

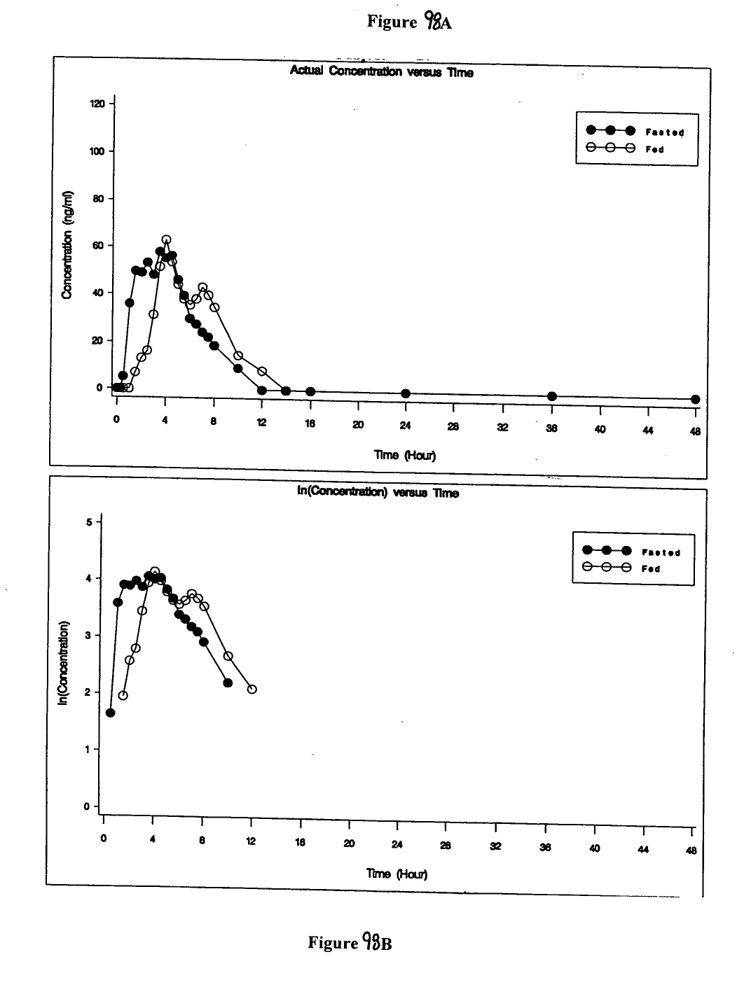

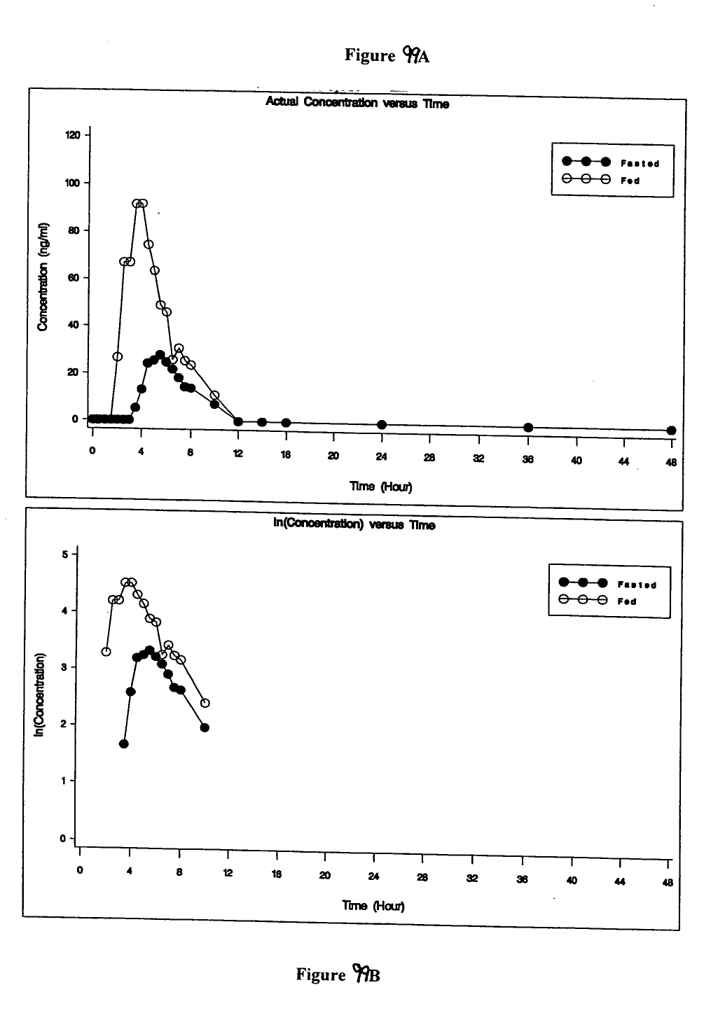

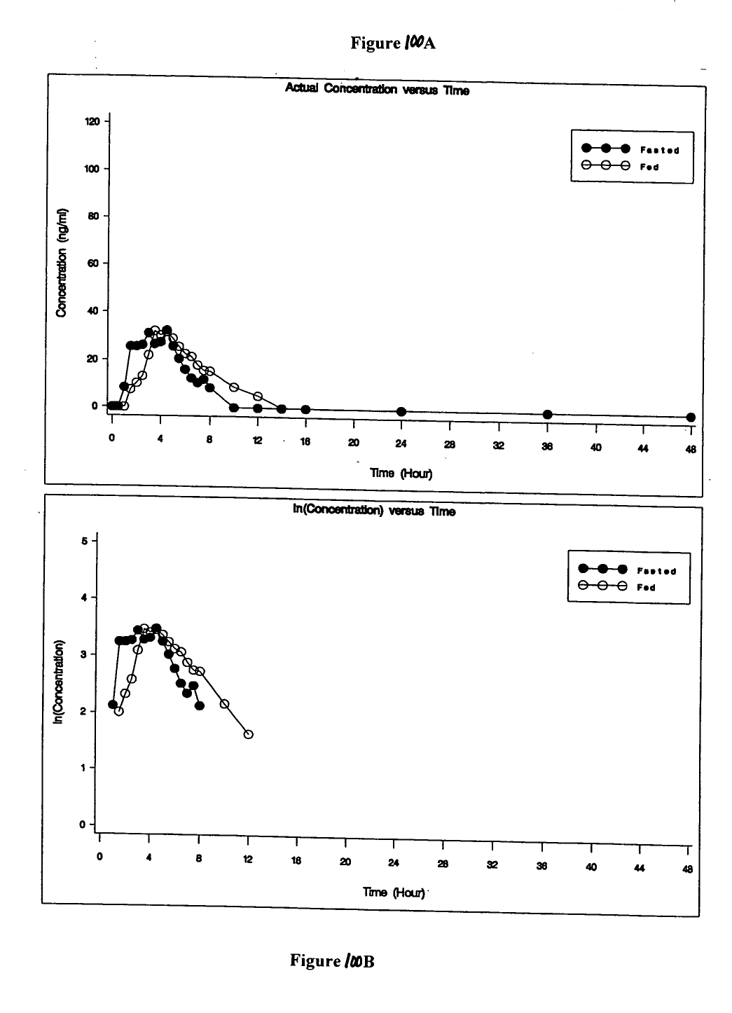

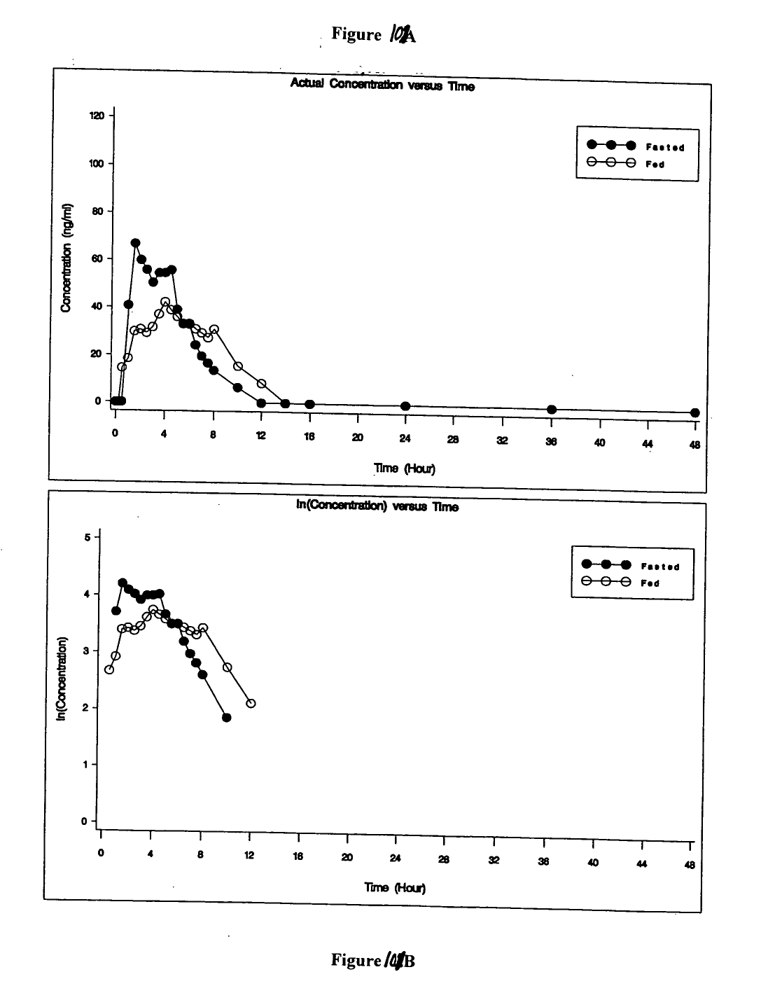

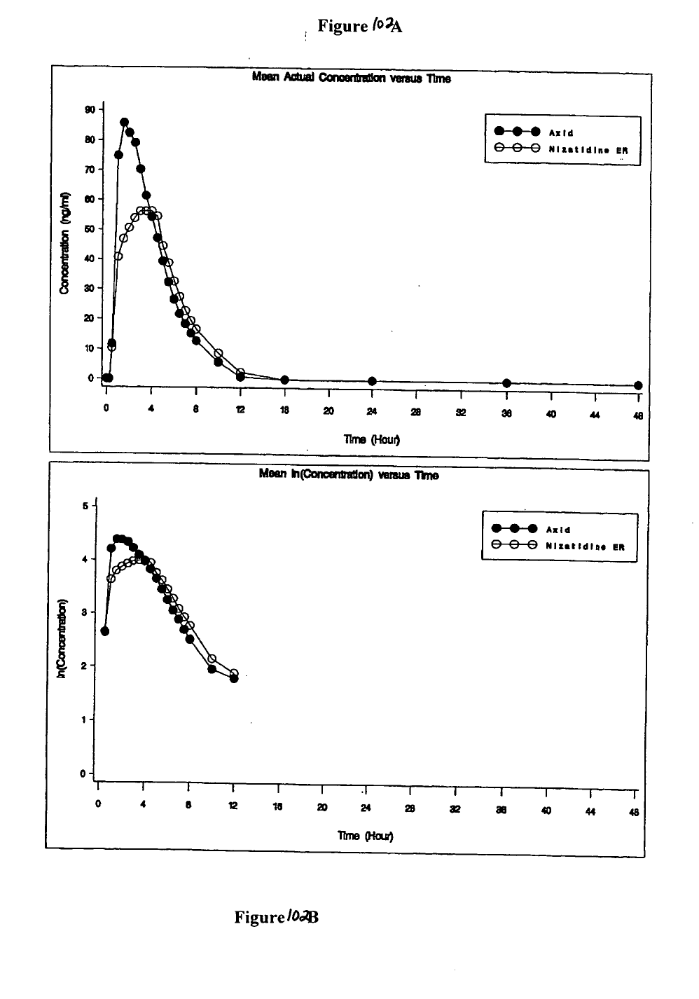

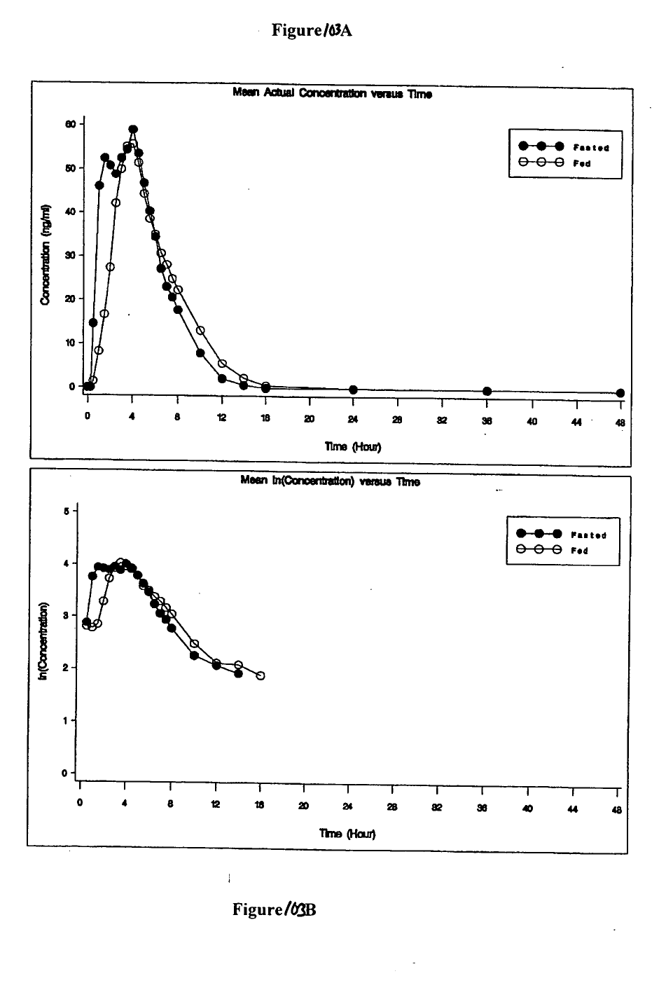

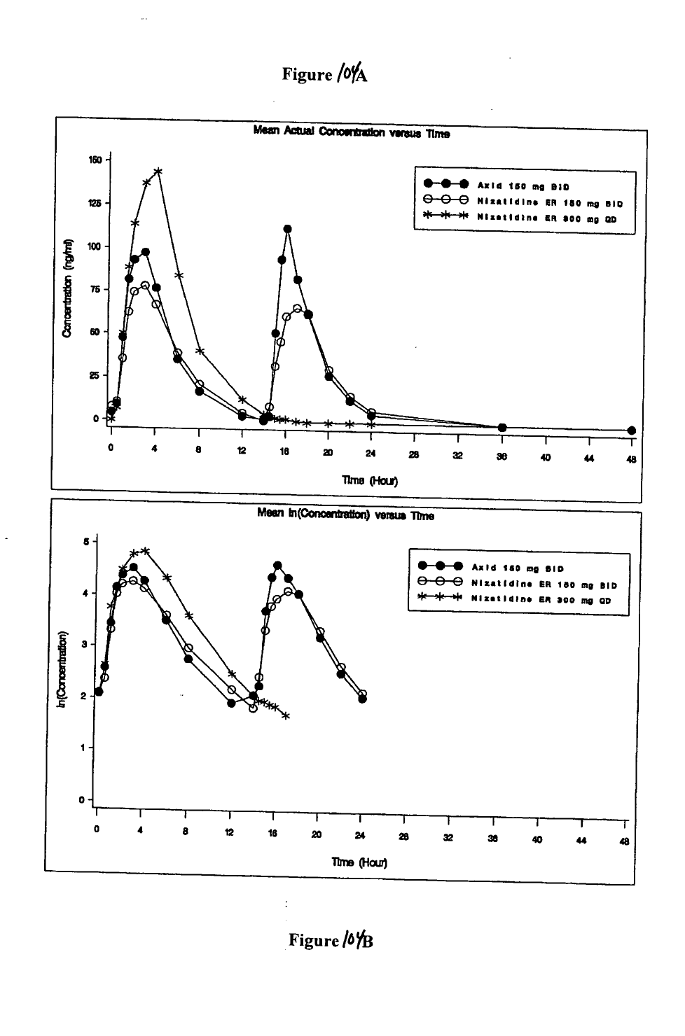

In embodiments, the invention is directed to an extended release (ER) oral dosage form comprising 150 mg of nizatidine, which after oral administration of a single one of said dosage forms in an adult human produces a blood plasma concentration of n-desmethylnizatidine ranging from 80 to 120% of the blood plasma concentration values per time period over the first eight hours shown in any one of FIGS. 56-101.

The following Examples illustrate the dosage formulations of the invention.

### EXAMPLES

Pulsatile Release capsules of nizatidine, a novel histamine H₂ receptor antagonist, comprise a mixture of two sets of beads: The first set is referred to as immediate release (IR) Beads and are designed to provide a loading dose by releasing all of the nizatidine within the first hour, preferably within the first 30 minutes. The second set is referred to as the Timed Pulsatile Release (TPR) Beads and are designed to release nizatidine in a ‘burst’ over a period of 2 hours after about 2-4 hour lag time. The TPR Beads are produced by applying an outer layer of pulse coating (comprising a blend of an enteric polymer such as HPMCP and a water insoluble polymer such as ethylcellulose) on IR Beads. The two sets of beads when filled into capsule shells at an appropriate ratio will produce the target circadian rhythm release profile required for maintaining drug plasma concentrations at potentially beneficial level when taken orally twice a day, after breakfast and dinner.

### Example 1

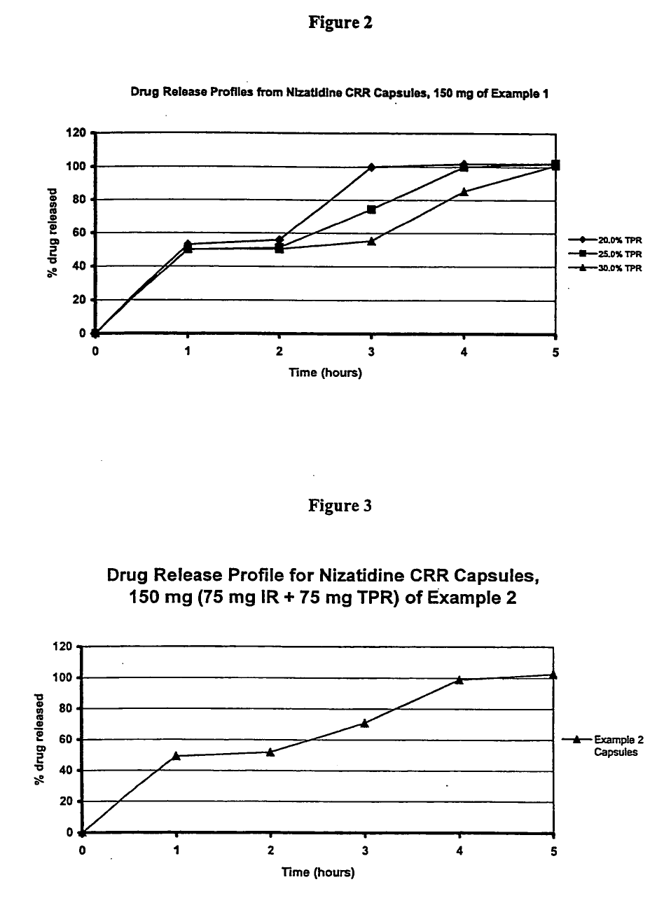

Nizatidine (5787.7 g) was slowly added to an aqueous solution of hydroxypropylcellulose such as Klucel LF (643.1 g) and mixed well. # 25-30 mesh sugar spheres (3700 g) were coated with the drug suspension in a Glatt fluid bed coater. The drug containing particles were dried, and a seal coat of Opadry Clear (2% w/w) was first applied. These drug containing IR Beads were provided with an outer membrane by spraying a solution of 1:1 blend of ethylcellulose and HPMCP plasticized with diethyl phthalate in 98/2 acetone/water in a fluid bed coater for a weight gain of approximately 39-40%. The coated particles are cured at 60° C. until the polymers were coalesced to produce TPR Beads. Pulsatile Release Nizatidine Capsules, 150 mg, were manufactured by filling 75 mg IR Beads and 75 mg TPR Beads into size 0 hard gelatin capsules using a MG Futura capsule filling equipment. The drug release testing was performed using USP Apparatus 2 (Paddles @ 50 rpm) in 0.1N HCl for 2 hours and subsequently at pH 6.8. The release profiles generated from Pulsatile Release Capsules comprising TPR Beads with different membrane coating levels are presented in FIG. 2.

### Example 2

Nizatidine (168 kg) was slowly added to an aqueous solution of hydroxypropylcellulose such as Klucel LF (18.6 kg) and mixed well. # 25-30 mesh sugar spheres (107.4 kg) were coated with the drug suspension in a Glatt fluid bed coater, equipped with a 32″ bottom spray Wurster insert. The drug containing particles were dried, and a seal coat of Opadry Clear (2% w/w) was first applied and dried in the Glatt fluid bed unit as a precautionary measure to drive off excessive surface moisture. These drug containing IR Beads were provided with an outer membrane by spraying a solution of 1:1 blend of ethylcellulose and HPMCP plasticized with diethyl phthalate in 98/2 acetone/water in a fluid bed coater for a weight gain of approximately 39-40%. The coated particles are cured at 60° C. for 4 hours to produce TPR Beads (batch size: 300 kg). Pulsatile Release Nizatidine Capsules, 150 mg, were manufactured by filling 75 mg IR Beads and 75 mg TPR beads into size 0 hard gelatin capsules. The drug release profile is shown in FIG. 3.

### Example 3

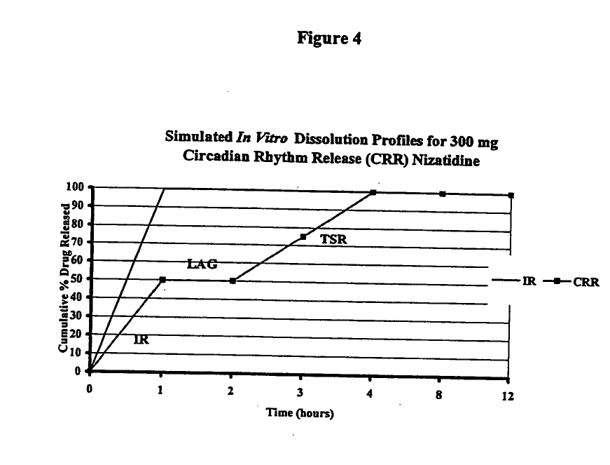

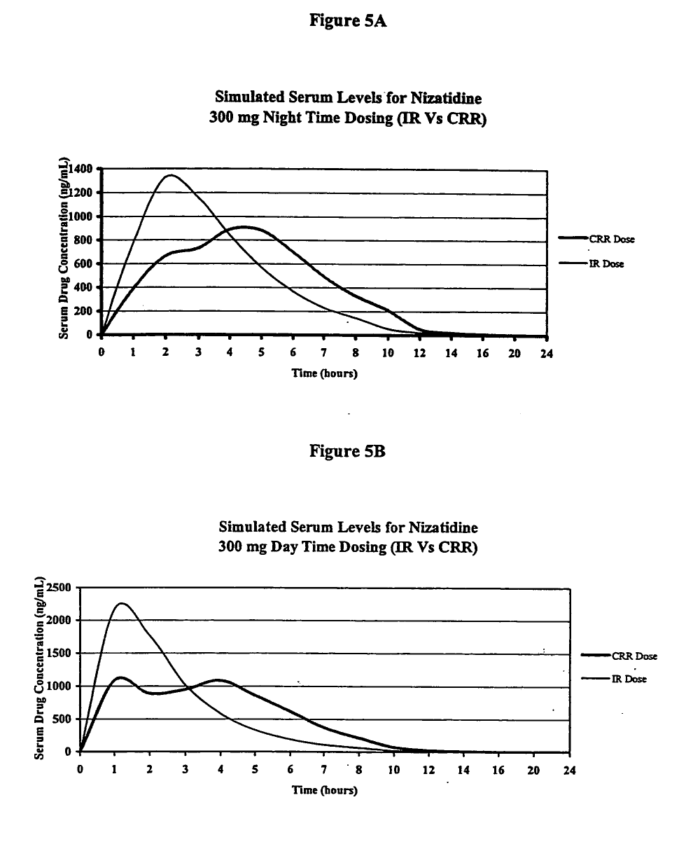

In order to assess the type of in vitro release profile needed to achieve a circadian rhythm effect under in vivo conditions, a modeling exercise was performed using the pharmacokinetic parameters for nizatidine. A diurnal variation in the pharmaco-kinetics of nizatidine has been reported by Jamali, A. et al., Journal of Clinical Pharmacology 35: 1071-1075 (1995), is incorporated in its entirety). A pharmaco-kinetic modeling was done separately to try to mimic both evening and day time results individually. Mean serum concentrations of nizatidine achieved in healthy volunteers were taken from the same literature. Theoretical in vitro dissolution profile (FIG. 4) as well as in vivo serum levels achieved during evening and daytime dosing, were simulated using the pharmaco-kinetic models developed. The advantages of a pulsatile dosage form are evident in attached FIG. 5 that compares simulated serum levels achieved with an immediate release dose of nizatidine versus the proposed pulsatile dose, being orally administered (a) in the evening and (b) during the daytime. The proposed dosage form is seen to give two pulses about 3.5-4.0 hours apart, maintaining an acceptable serum concentration for about 6.0-8.0 hours in the body, irrespective of whether evening or day time dosing is considered. Thus, the presence of the TPR portion should ideally sustain enough drug in the body right around midnight when literature has reported a circadian rhythm to gastric acid secretion and increased severity of symptoms associated with GERD.

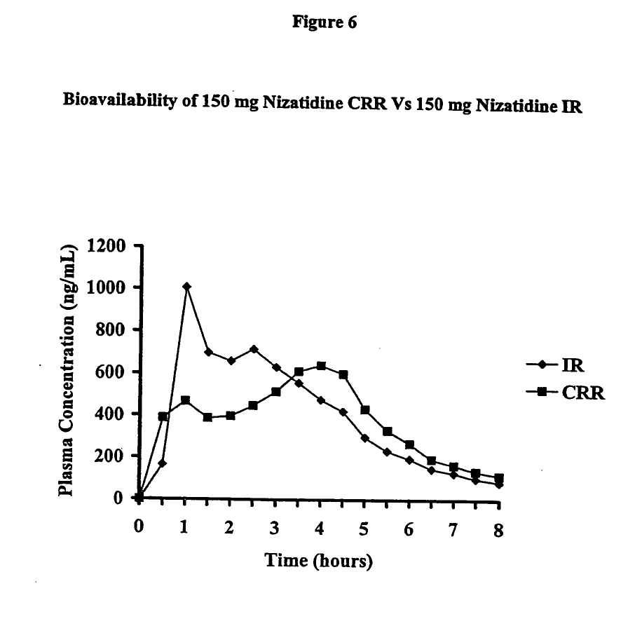

Clinical supplies, nizatidine pulsatile Capsules, 150 mg, comprising of 75 mg IR and 75 mg TPR Beads were manufactured following Example 1, by filling hard gelatin size# 0 capsules. FIG. 6 shows the plasma concentration profile (a bimodal display) achieved in a healthy volunteer when dosed after dinner.

### Example 4

The nizatidine pulsatile Capsules prepared in Example 3 were utilized in two randomized, double-blind, comparative, multiple dose efficacy studies. The clinical efficacy studies included a total of 428 subjects with GERD who were treated with the subject nizatidine Capsules and 215 treated with placebo. For the purpose of summarizing the nizatidine Capsules efficacy data, the two randomized, double-blind, comparative, multiple dose efficacy studies were conducted under identical protocols during the same time period, and identical case report forms were used for both studies. Clinical studies were designed to assess the safety and efficacy of nizatidine Capsules 150 mg bid, nizatidine Capsules 300 mg and placebo in adult subjects with clinical symptoms and endoscopic evidence of erosive and ulcerative GERD. Subjects meeting the entry criteria were randomized to receive one of the three treatments and began taking study medication in the evening on Day 0. Study medication was taken for up to 12 weeks, with follow-up visits at weeks 3, 6 and 12.

The results of the combined efficacy analyses indicated that clinically and statistically significant healing of erosive esophagitis with associated symptom relief was produced by the nizatidine Capsules administered either as individual doses (150 mg bid) or as a single evening dose of 300 mg. For the nizatidine Capsule 150 mg bid, statistically significant and clinically meaningful overall healing was also demonstrated. Subjects treated with nizatidine Capsules bid had a significantly greater mean change from baseline in their endoscopy grade and there was a notable trend toward efficacy in the proportions of subjects who had ≧2 points improvement in baseline endoscopy grade compared to those treated with placebo. Subjects treated with nizatidine Capsules 300 mg qd also had a greater mean change from baseline in their endoscopy grade. Based on subject rated evening symptom scores, statistically significant and clinically meaningful evening relief of heartburn, regurgitation and retrosternal pain was demonstrated during the first week of treatment for both nizatidine Capsules 150 mg bid and nizatidine Capsules 300 mg qd. Based on Investigator-rated evening symptom scores, treatment with nizatidine Capsules 150 mg bid was significantly superior to placebo at Week 12 for heartburn and regurgitation, and there was a trend toward efficacy for retrosternal pain. Treatment with nizatidine Capsules 300 mg qd was significantly superior to placebo at Week 12 for heartburn, regurgitation and retrosternal pain. Based on Investigator rated daytime symptom scores, treatment with nizatidine Capsules 150 mg bid was significantly superior to placebo at Week 12 for daytime heartburn and retrosternal pain. Nizatidine Capsules 300 mg qd was significantly superior to placebo at Week 12 for daytime retrosternal pain. Subjects treated with nizatidine Capsules 150 mg bid used significantly less antacid tablets per day than did those treated with placebo (P<0.001).

The study conclusion was as follows:

“Overall, in subjects with endoscopically proven GERD, nizatidine CR administered in doses of either 150 mg bid or 300 mg qd was effective in healing esophageal erosions and in relieving GERD symptoms.”

### Example 5

Cimetidine was slowly added to an aqueous solution of polyvinylpyrrolidone and mixed well. # 25-30 mesh sugar spheres were coated with drug solution in a Glatt fluid bed granulator. The drug containing pellets were dried, and a seal coat of Opadry Clear (2% w/w) was first applied. The inner polymer coating was applied to the active particles by spraying an aqueous dispersion of ethylcellulose (aquacoat® ECD-30 with dibutyl sebacate as the plasticizer to produce intermediate release (IntR) Beads. An outer coating formulation was prepared by mixing two separate aqueous dispersions of Eudragit L30D plasticized with acetyl tri-n-butyl citrate and Aquacoat ECD-30 (an aqueous dispersion of ethylcellulose) plasticized with dibutyl sebacate. The combined coating formulation was sprayed onto the ethylcellulose coated IntR Beads. The coated particles are cured at 60° C. until the polymers were coalesced to produce TSR Beads. The finished SR and TSR Beads were tested for in vitro dissolution properties using USP Dissolution Apparatus 2 at a paddle speed of 50 rpm. The beads were dissoluted using a three-stage dissolution medium, i.e., first 2 hours in 0.1N HCl, next 2 hours at pH 4.0 and then at pH 6.8 for additional 14 hours, the pH of the medium being changed by adding a pH modifier. The results obtained are presented in Table 1. The dissolution results show that there is a lag time of about four hours followed by sustained release occurring over a period of 12-14 hours for the TSR Beads.

TABLE 1
Dissolution Data for SR and TSR Beads of Example 4

| | | TSR Beads |
|---|---|---|
| | SR Beads | SR Coating (1.8% w/w)/ |
| Time, hours | SR Coating (1.8% w/w) | TSR Coating (15% w/w) |
| 1.0 | 0.2 | 0 |
| 2.0 | 0.1 | 0 |
| 3.0 | 0.5 | 0.5 |
| 4.0 | 0.2 | 0.4 |
| 5.0 | 15 | 10 |
| 6.0 | 42 | 24 |
| 8.0 | 71 | 47 |
| 10.0 | 85 | 62 |
| 12.0 | 93 | 72 |
| 14.0 | 98 | 78 |
| 16.0 | 103 | 86 |

### Example 6

The nizatidine pulsatile Capsules prepared in Example 3 were utilized it three open-label pharmacokinetic/bioavailability studies. The pharmacokinetic/bioavailability studies consisted of two single dose studies and one multiple dose study that also evaluated gastric pH. The pharmacokinetic/bioavailability studies included a total of 68 healthy volunteers who received nizatidine CR.

In each study, blood samples were obtained over the 48 hours after the last dose of nizatidine CR in each treatment period. Plasma samples were assayed for nizatidine and N-desmethylnizatidine (the principal metabolite of nizatidine) at MDS Pharma Services (Montréal, Canada) using a high pressure liquid chromatography (HPLC) assay with ultraviolet detection. Each study had a 7 day washout between treatments.

The first study investigated the bioavailability of nizatidine following the administration of single oral doses of nizatidine CR 150 mg and Axid® 150 mg in fasted normal healthy male subjects. After an overnight fast, 24 subjects where randomly assigned to and received a single dose of extended release nizatidine 150 mg (nizatidine ER) or Axid 150® mg, with a 7 day washout period between each dose. Subjects continued to fast for 4 hours after each dose and remained at the clinical research unit for at least 48 hours.

Blood samples for the determination of plasma concentrations of nizatidine and n-desmethylnizatidine (the primary nizatidine metabolite) were obtained immediately before each dose and for set periods during the 48-hour period after each dose. The following pharmacokinetic variables were determined from the nizatidine and n-desmethylnizatidine plasma concentration-time curves for each subject: Kₑₗ, the apparent terminal elimination rate constant; AUC₀₋₁₀, the area under the plasma concentration-time curve from dosing until the last measurable plasma concentration (AUCₗₐₛₜ); AUC_{0-inf}, the area under the plasma concentration-time curve extrapolated to infinity; Cₘₐₓ, the maximum observed plasma concentration; tₘₐₓ, the time to the maximum observed plasma concentration; t_{1/2}, the apparent plasma terminal elimination half life; and t_{lag}, the absorption lag time (delay between drug administration and the beginning of absorption).

FIGS. 7A and 7B summarize the plasma concentration profile achieved in fasted healthy male subjects after the administration of nizatidine and Axid®. FIGS. 10-32 summarize the plasma levels of Nizatidine in representative normal healthy male subjects, following oral administration of Nizatidine Pulsatile Capsule, 150 mg versus Axid® 150 mg. FIGS. 56-78 summarize the plasma levels of n-Desmethylnizatidine in representative normal healthy male subjects, following oral administration of Nizatidine Pulsatile Capsule, 150 mg versus Axid® 150 mg.

Following administration, nizatidine and n-desmethylnizatidine levels rapidly increased, and the increase was more rapid for subjects who received Axid®. The pharmacokinetic profile of nizatidine ER was distinctly different from that of Axid®, displaying characteristics of an extended release formulation. The lower maximum plasma nizatidine concentration observed for nizatidine ER (543 ng/mL after first pulse and 513 ng/mL after the second pulse) were expected given the pulsatile release property of the formulation. While Cₘₐₓ for nizatidine ER was reduced by more 20%, preferably more than 30%, even more preferably more than 42%, the Tₘₐₓ increased about 1.0 to 2.0 times longer, preferably 1.2 to 1.8 times longer, even more preferably 1.6 times longer, with the nizatidine bimodal release formulation (3.15) when compared to Axid® (1.67). Of the 23 evaluable subjects, 17 (74%) had at least one nizatidine plasma concentration that was at least 490 ng/mL and 14 (74%) of 19 subjects with a bimodal nizatidine plasma concentration-time profile had both peak plasma nizatidine concentrations of at least 430 ng/mL.

### Example 7

The second study evaluated whether the co-administration of food (standard high-fat meal) affects the bioavailability and/or release profile of nizatidine CR 150 mg in normal healthy male subjects. After an overnight fast of at least 10 hours, 24 subjects were given a single dose of nizatidine ER 150 mg on two occasions, with at least seven days between each dose, in the fed (after a standard high-fat meal) and fasted states. Blood samples for the determination of plasma concentrations of nizatidine and n-desmethylnizatidine were obtained immediately before each dose and for set periods during the 48-hour period after each dose. Subjects were not allowed to eat for 4 hours after each dose and remained at the clinical research unit for at least 48 hours.

The following pharmacokinetic variables were determined from the nizatidine and n-desmethylnizatidine plasma concentration-time curves for each subject: Kₑₗ, the apparent terminal elimination rate constant; AUC₀₋₁₀, the area under the plasma concentration-time curve from dosing until the last measurable plasma concentration (AUCₗₐₛₜ); AUC_{0-inf}, the area under the plasma concentration-time curve extrapolated to infinity; Cₘₐₓ, the maximum observed plasma concentration; tₘₐₓ, the time to the maximum observed plasma concentration; t_{1/2}, the apparent plasma terminal elimination half life; and t_{lag}, the absorption lag time (delay between drug administration and the beginning of absorption).

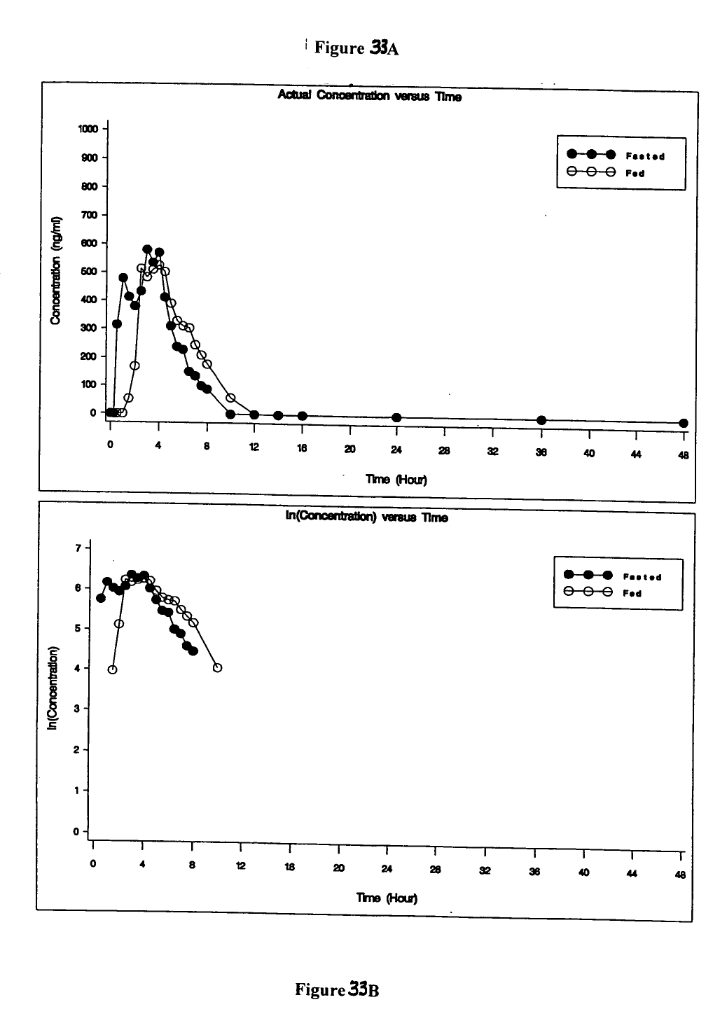

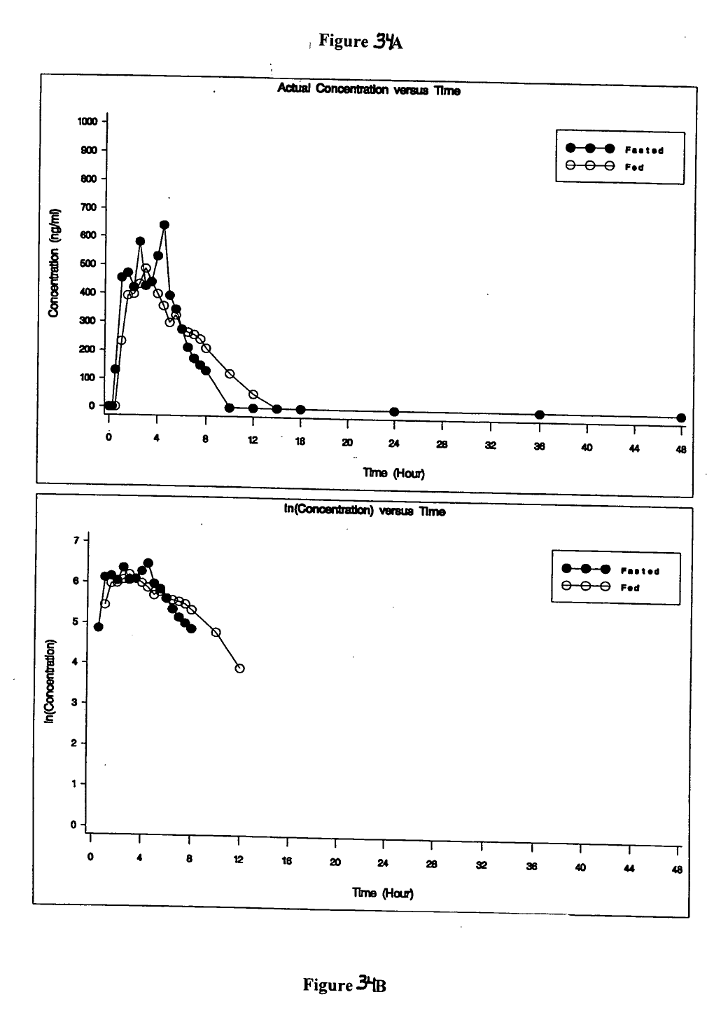

FIGS. 8A and 8B summarize the plasma concentration profile achieved in healthy male subjects after the administration of nizatidine ER 150 mg in fed or fasted conditions. FIGS. 33-55 summarize the plasma levels of Nizatidine in representative normal healthy male subjects, following oral administration of Nizatidine Pulsatile Capsule in fed versus fasted conditions. FIGS. 79-101 summarize the plasma levels of n-Desmethylnizatidine in representative normal healthy male subjects, following oral administration of Nizatidine Pulsatile Capsule in fed versus fasted conditions.

Nizatidine levels rapidly increased after administration. The increase was more rapid for 21 of the 22 fasted subjects who had a nizatidine profile characterized by two peak concentrations which were about 0 to about 5 hours apart, preferably 1.0 to about 4.0 hours, more preferably about 2 to about 2.5 hours apart. In contrast, for 13 of the 22 fed subjects, their profile was characterized by a single peak concentration that was approximately 40-65 ng/mL lower than either peak concentration for fasted subjects and which occurred at approximately the same time as the second peak concentration for fasted subjects. The effect of food was to delay the start of absorption of nizatidine by an average of 0.8 hours and slow the rate of nizatidine absorption, as reflected in a mean tₘₐₓ that was longer by 0.9 hours.

Further, 16 (76%) of the 21 fasted subjects with a bimodal nizatidine profile had both peak plasma nizatidine concentrations of at least 350 ng/mL, more preferably at least 390 ng/mL, even more preferably at least 430 ng/mL. While fasting, 18 (82%) of the 22 evaluable subjects had at least one nizatidine plasma concentration that was at least 350 ng/mL, more preferably at least 390 mg/L, even more preferably at least 490 ng/mL. Among fed subjects, 16 (73%) had at least one nizatidine plasma concentration that was at least 430 ng/mL, and 11 (50%) had at least one nizatidine plasma concentration that was at least 490 ng/mL.

### Example 8

A pharmacokinetic-pharmacodynamic study was conducted that evaluated the bioavailability of nizatidine following multiple dosing with extended release nizatidine (nizatidine CR) 150 mg bid and nizatidine CR 300 mg qd relative to that of immediate release nizatidine (Axid®) 150 mg bid. The study also evaluated the effects of these dosages on infra-gastric acid pH and the relationship between infra-gastric pH and nizatidine plasma concentrations.

The study was a single center, open-label, randomized, 3-period crossover study in normal, healthy male or female subjects. Subjects were randomly assigned to one of six treatment sequences consisting of a different ordering of the three treatments. Subjects received one nizatidine CR 150 mg tablet given twice daily, two nizatidine CR 150 mg tablets given once daily, or one Axid® 150 mg pulvule given twice daily, for five days. More specifically, subject received, 10 doses of nizatidine CR 150 mg and Axid® 150 mg, administered daily at 6:00 pm and 8:00 am starting with the 6:00 pm dose on day 1. Subjects also were to receive 5 daily doses of nizatidine CR 300 mg starting with the 6:00 pm dose on day 1. Each dose of study medication was administered 90 minutes after the start of breakfast or dinner (a standard high fat meal).

At least 24 hours before the first dose of study medication in the first treatment period, each subject had a pH probe inserted nasogastrically for infra-gastric pH monitoring (the instrument used was the GERD√ ambulatory pH recording system). Monitoring was to continue for 24 hours until the first dose of study medication was administered. Intragastric pH level monitoring also was to start on day 5 of each treatment period at the time the 6:00 pm dose of study medication was administered and was to continue for 24 hours.

In each treatment period, blood samples were collected for the determination of plasma nizatidine concentrations. There was a seven day washout between each treatment period. All procedures performed before and during the first treatment period were to be performed before and during the second and third treatment periods, except the 24-hour gastric pH monitoring, which was not performed before the start of the second or third treatment periods.

Using the GERD√ Analysis Software, data from the pH monitor were downloaded for statistical analysis. The pH monitor recorded pH values every 10 seconds over the 24-hour monitoring period. For each subject the average pH over each 15-minute interval was computed. These 15-minute averages were used to estimate the following pharmacodynamic parameters for each subject: the area under the gastric pH-time curve (pH AUC from hour 0 to hour 14, from hour 14 to hour 24, and from hour 0 to hour 24) above a pH of 0 calculated by the linear trapezoidal rule, the maximum observed gastric pH, the average pH, the percentage of time the gastric pH was greater than 3 and greater than 4 (t_{pH>3}, t_{pH>4}) over the 0-24, 0-14, and hour intervals, the time (hours) to the maximum observed pH, and AUC₀₋₁₄, AUC₀₋₁₄, AUC₁₄₋₂₄ and AUC₀₋₂₄.

Twenty-four subjects were screened and 22 of them received at least one dose of study medication. Of the subjects, 21 were included in the pharmacokinetic analyses, and 20 had complete data for use in the analyses of changes in pharmacodynamic parameters.

Following the 6:00 pm dose of study medication, pH peaked around 3 hours later, shortly after the peak nizatidine plasma concentrations which occurred at 1.8 to 2.6 hours after the last morning and evening doses of study medication. Mean pH values for nizatidine CR 300 mg were consistently greater than those of Axid® during hours 3 to 12. After this time, when nizatidine plasma concentrations for nizatidine CR 300 mg were for the most part below the limit of detection, the mean intragastric pH vales remained close to the baseline values. Generally, the mean pH values for subjects who received nizatidine CR 150 mg or Axid® were comparable over the 24-hour dosing period.

In the nizatidine CR 300 mg group during hours 3 to 12, there was a bimodal pattern of improvement in gastric pH which remained above baseline. During hours 12 to 24 in the nizatidine CR 150 mg group, the pulsatile formulation of nizatidine served to maintain mean gastric pH above baseline throughout most of the period, in contrast to the Axid® group where mean pH values fell below baseline after approximately 18 hours.

The significant, differences among treatment groups for pH AUC, pHₜ, t_{pH>3}, t_{PH>4}, whether based on actual values or changes from baseline, reflected higher values for nizatidine CR 300 mg compared to either nizatidine CR 150 mg or Axid® during the 0 to 14 hours after the evening dose of study medication and lower values for nizatidine CR 300 mg compared to either nizatidine CR 150 mg or Axid® during the 14 to 24 hours after the evening dose of study medication. During the 0 to 14 hour time period, the mean values for nizatidine CR 150 mg and Axid® were similar. For the 14 to 24 hour time period, nizatidine CR 150 mg maintained gastric pH over 3.0 for 42% of the time compared to 39% for Axid, and maintained a gastric pH over 4.0 for 27% of the time compared to 23% for Axid®.

For nizatidine CR 150 mg, nizatidine CR 300 mg, and Axid®, there was no significant linear relationship between the nizatidine AUC (AUC₀₋₁₄, AUC₁₄₋₂₄, and AUC₀₋₂₄) and either pH AUC (pH AUC₀₋₁₄, pH AUC₁₄₋₂₄, and pH AUC₀₋₂₄) or t_{pH>3} (t_{pH>3 0-14}) t_{PH>-4-24}, and t_{pH>3 0-24}).

FIGS. 9A and 9B show the plasma concentration profile achieved in healthy subjects after the administration of nizatidine CR 150 mg bid, nizatidine CR 300 mg qd and Axid®. After the last dose of nizatidine CR 300 mg all subjects had peak nizatidine levels that exceeded 350 ng/mL, more preferably exceeding 420 ng/mL, even more preferably exceeding 490 ng/mL. After their last evening dose of nizatidine CR 150 mg all subjects had peak nizatidine levels that exceeded 350 ng/mL, more preferably exceeding 420 ng/mL, even more preferably exceeding 490 ng/mL. After the last morning dose, all subjects had peak nizatidine levels that exceeded 310 ng/mL, more preferably exceeding 370 ng/mL, even more preferably exceeding 430 ng/mL and 19 (90%) had peak nizatidine levels that exceeded 350 ng/mL, more preferably exceeding 420 ng/mL, even more preferably exceeding 490 ng/mL.

The pharmacokinetic profile of nizatidine CR 150 mg revealed that most subjects had a single peak nizatidine concentration. However, the bioavailability of nizatidine was not affected based on total and partial AUC comparisons.

### Example 9

Two prokinetic studies were conducted to evaluate whether nizatidine accelerates gastric emptying in subjects with GERD, as well as the safety of nizatidine CR 150 mg and nizatidine CR 30 mg. The studies were randomized, double-blind crossover studies with a qualifying single-blind placebo phase in 85 human male or female subjects aged 18 years or older with at least a 3-month history of diagnosed GERD.

After an initial screening visit for medical history (including medications), physical examination, vital signs (blood pressure and heart rate, clinical laboratory tests, and symptom assessments, eligible subjects received one single-blind dose of placebo. A standardized meal was given one hour after dosing. After ingestion of the meal, a scintigraphic gastric emptying test was performed with anterior and posterior images taken at designated intervals. Each subject was classified as having “normal” or “abnormal” gastric emptying. For enrollment purposes, abnormal gastric emptying was defined as percent gastric retention at 4 hours post-meal of greater than 10% percent. For analysis purposes, abnormal gastric emptying was later modified to be defined as percent retention at 2 hours post-meal of greater than 40% and or percent retention at 4 hours post-meal of greater than 6.3%.

On the third visit, subjects were given one of two double-blind treatment sequences: AB or BA, where subjects received a single oral dose of Treatment A (nizatidine CR 150 mg) or Treatment B (nizatidine CR 300 mg, as 2×150 mg capsules). At the fourth visit, subjects received the treatment they did not receive on the third visit. The third and fourth visit were separated by a minimum of 48 hours up to a maximum of 5 days. At the third and fourth visits, the standardized meal was given one hour before dosing and the scintigraphic emptying test was performed following completion of the meal.

For percent gastric retention, half-life for gastric emptying, and the lag phase, the three treatments (nizatidine CR 150 mg, nizatidine CR 300 mg and placebo) were compared in a pairwise fashion using Wilcoxon signed-rank tests. Repeated measures analyses were also performed using a model including treatment, sequence and period as fixed effects.

In the first study, 23 normal and 23 abnormal subjects were analyzed. In the second study, 39 abnormal subjects randomized and analyzed. The analysis of percent gastric retention at 4 hours post-meal for the evaluable population, i.e. abnormal subjects who had gastric emptying studies at visits 2, 3, and 4, showed that nizatidine CR 150 mg was significantly superior to placebo at 4 hours post-meal (11.4% vs. 13.7%, p=0.026) and that nizatidine CR 300 mg was significantly superior to placebo at both 3 hours post-meal (22.4% vs. 27.6%, p=0.030) and 4 hours post-meal (8.8% vs. 13.7%, p<0.001).

The percent gastric retention at 4 hours post-meal in the intent-to-treat (ITT) population, i.e. all subjects who had a gastric emptying study at the second visit, and at visit 3 or 4 or both, showed that nizatidine CR 150 mg was significantly superior to placebo in gastric emptying status (68.3% normal vs. 50.0% normal, p=0.011) and in lag time (0.57 hours vs. 0.47 hours, p=0.043). Nizatidine CR 300 mg was significantly superior to placebo in percent gastric retention at 4 hours post-meal (6.6% vs. 8.0%, p=0.017) and in gastric emptying (72% normal vs. 51.2% normal, p=0.04).

The subgroup of percent gastric retention in diabetic subjects was analyzed as well and nizatidine CR 300 mg was found to be significantly superior to placebo at 3 hours (16.0% vs. 26.2%, p=0.033) and at 4 hours (9.5% vs. 14.8%, p=0.037). There were no significant differences observed between nizatidine CT 150 mg and placebo.

1. A histamine H₂ antagonist pharmaceutical dosage form providing a bi-modal pulsatile release profile comprising:
a. immediate release (IR) beads comprising an active-containing core particle; and
b. timed pulsatile release (TPR) beads, wherein said TPR beads comprise:
i. an active-containing core particle; and
ii. a pulse coating surrounding said core,
wherein said IR beads provide a therapeutically effective amount of active to treat gastric acid secretions and the TPR beads provide a delayed dose of active which provides a therapeutically effective amount of active to treat midnight GERD.

2. A pharmaceutical dosage form as defined in claim 1, wherein said histamine H₂ receptor antagonist is selected from the group consisting of nizatidine, cimetidine, ranitidine, and famotidine and derivatives thereof.

3. A pharmaceutical dosage form as defined in claim 1, wherein said timed pulsatile release (TPR) beads when tested in a USP Type II apparatus at 50 rpm using a 2-stage dissolution medium (first 2 hours and 700 ml 0.1 N HCl at 37° C. followed by a dissolution in a pH of 6.8 obtained by the addition of 200 ml of pH modifier) exhibits a dissolution profile substantially corresponding to the following pattern:
after 2 hours, about 0-25% of the total active is released;
after 3 hours, about 15-80% of the total active is released; and after 4 hours, not less than 60% of the total active is released.

4. A pharmaceutical dosage form as defined in claim 3, wherein said dissolution profile substantially corresponds to the following pattern:
after 2 hours, about 0-15% of the total active is released;
after 3 hours, about 20-65% of the total active is released; and
after 4 hours, not less than 70% of the total active is released.

5. A pharmaceutical dosage form as defined in claim 4, wherein said dissolution profile substantially corresponds to the following pattern:
after 2 hours, about 0-5% of the total active is released;
after 3 hours, about 30-50% of the total active is released; and
after 4 hours, not less than 80% of the total active is released.

6. A pharmaceutical dosage form as defined in claim 1, wherein said pulse coating comprises a water insoluble polymer and an enteric polymer.

7. A pharmaceutical dosage form as defined in claim 6, wherein said enteric polymer is selected from the group consisting of esters of cellulose, polyvinyl acetate phthalate, pH-sensitive methacrylic acid-methylmethacrylate copolymers, shellac and derivatives thereof.

8. A pharmaceutical dosage form as defined in claim 7, wherein said enteric polymer is selected from the group consisting of cellulose acetate phthalate, hydroxypropyl methylcellulose phthalate, hydroxypropyl methylcellulose succinate and combinations thereof.

9. A pharmaceutical dosage form as defined in claim 6, wherein at least one of said polymers further comprises a plasticizer.

10. A pharmaceutical dosage form as defined in claim 9, wherein said plasticizer is selected from the group of triacetin, tributyl citrate, tri-ethyl citrate, acetyl tri-n-butyl citrate, diethyl phthalate, dibutyl sebacate, polyethylene glycol, polypropylene glycol, castor oil and acetylated mono- and di-glycerides and mixtures thereof.

11. A dosage form as defined in claim 6, wherein said water insoluble polymer and said enteric polymer are present in said pulse coating at a ratio from about 4:1 to about 1:2.

12. A dosage form as defined in claim 11, wherein said ratio of water insoluble polymer to enteric polymer is from about 2:1 to about 1:2.

13. A dosage form as defined in claim 11, wherein said water insoluble polymer is ethylcellulose and said enteric polymer is hydroxypropyl methylcellulose phthalate.

14. A dosage form as defined in claim 13, wherein said ratio is about 1:1.

15. A dosage form as defined in claim 1, wherein said IR beads release substantially all of the active contained therein within the first hour after administration of the dosage form.

16. A dosage form as defined in claim 1, wherein said IR beads and TPR beads are present in a ratio from about 3:1 to about 1:3.

17. A dosage form as defined in claim 16, wherein said IR beads and TPR beads are present in a ratio from about 2:1 to about 1:2.

18. A dosage form as defined in claim 1, wherein the total weight of the coating on the TPR beads is about 10-60 weight % based on the total weight of the TPR beads.

19. A method for the preparation of the dosage form of claim 1, comprising the steps of:
a. preparing an active-containing core to form IR beads;
b. coating a fraction of the IR beads with a water insoluble polymer and an enteric polymer to form TPR beads; and
c. filling capsules with IR beads and TPR beads at a ratio from about 3:1 to about 1:3.

20. The method of claim 19, wherein said active-containing core is produced by coating a particle selected from the group consisting of non-pareil seeds, acidic buffer crystals and alkaline buffer crystals with a water soluble film-forming composition comprising nizatidine and a polymeric binder.

21. The method of claim 19, wherein said active-containing core is produced by granulating and milling and/or by extruding and spheronizing a polymer composition containing nizatidine.

22. A pulsatile release nizatidine dosage form comprising:
a. immediate release (IR) beads comprising a nizatidine-containing core particle; and
b. timed pulsatile release (TPR) beads, wherein said TPR beads comprise:
i. a nizatidine-containing core particle;
ii. a pulse coating surrounding said core, said pulse coating comprising ethylcellulose and an enteric polymer;
wherein said TPR beads when tested in a USP type II apparatus at 50 rpm using a 2-stage dissolution medium (first 2 hours and 700 ml 0.1 N HCl at 37° C. followed by a dissolution in a pH of 6.8 obtained by the addition of 200 ml of pH modifier) exhibits a dissolution profile substantially corresponding to the following pattern:
after 2 hours, about 0-25% of the total nizatidine is released;
after 3 hours, about 15-80% of the total nizatidine is released; and
after 4 hours, not less than 60% of the total nizatidine is released.

23. A pharmaceutical dosage form as defined in claim 22, wherein said dissolution profile substantially corresponds to the following pattern:
after 2 hours, about 0- 15% of the total nizatidine is released;
after 3 hours, about 20-65% of the total nizatidine is released; and
after 4 hours, not less than 70% of the total nizatidine is released.

24. A pharmaceutical dosage form as defined in claim 22, wherein the dissolution profile substantially corresponds to the following pattern:
after 2 hours, about 0-5% of the total nizatidine is released;
after 3 hours, about 30-50% of the total nizatidine is released; and
after 4 hours, not less than 80% of the total nizatidine is released.

25. A pharmaceutical dosage form as defined in claim 22, wherein the core particle is a non-pareil sugar seed coated with nizatidine and a polymeric binder, or the core particle is prepared by granulating and milling and/or by extruding and spheronizing a polymer composition containing nizatidine, to form a core particle containing nizatidine.

26. A pharmaceutical dosage form as defined in claim 22, wherein said enteric polymer is selected from the group consisting of esters of cellulose, polyvinyl acetate phthalate, pH-sensitive methacrylic acid-methylmethacrylate copolymers, shellac and derivatives thereof.

27. A pharmaceutical dosage form as defined in claim 26, wherein said enteric polymer is selected from the group consisting of cellulose acetate phthalate, hydroxypropyl methylcellulose phthalate, hydroxypropyl methylcellulose succinate and combinations thereof.

28. A pharmaceutical dosage form as defined in claim 22, wherein said pulse coating further comprises a plasticizer.

29. A pharmaceutical dosage form as defined in claim 28 wherein said plasticizer is selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil and acetylated mono- and di-glycerides and mixtures thereof.

30. A pharmaceutical dosage form as defined in claim 22, wherein said ethylcellulose and said enteric polymer are present in said pulse release coating at a ratio from about 4:1 to about 1:2.

31. A pharmaceutical dosage form as defined in claim 30, wherein said ratio of ethylcellulose to enteric polymer is from about 2:1 to about 1:2.

32. A pharmaceutical dosage form as defined in claim 31, wherein said enteric polymer is hydroxypropyl methylcellulose phthalate.

33. A pharmaceutical dosage form as defined in claim 32, wherein said ratio is about 1:1.

34. A dosage form as defined in claim 22, wherein said IR beads release substantially all of the nizatidine contained therein within the first hour after administration of the dosage form.

35. A pharmaceutical dosage form as defined in claim 22, wherein said IR beads and TPR beads are present in a ratio from about 3:1 to about 1:3.

36. A pharmaceutical dosage form as defined in claim 35, wherein said IR beads and TPR beads are present in a ratio from about 2:1 to about 1:2.

37. A pharmaceutical dosage form as defined in claim 22, wherein the total weight of the coating on the TPR beads is about 10-60 weight % based on the total weight of the TPR beads.

38. A pharmaceutical dosage form as defined in claim 22, wherein said IR beads contain a total of about 50-100 mg of nizatidine and said TPR beads contain a total of about 50-100 mg of nizatidine.

39. A method for the preparation of the dosage form of claim 1, comprising the steps of:
a. preparing a nizatidine-containing core to form IR beads;
b. coating a fraction of the IR beads with a mixture of plasticized ethylcellulose and an enteric polymer to form TPR beads; and
c. filling capsules with IR beads and TPR beads at a ratio from about 3:1 to about 1:3.

40. The method of claim 39, wherein said nizatidine-containing core is produced by coating a particle selected from the group consisting of non-pareil seeds, acidic buffer crystals and alkaline buffer crystals with a water soluble film-forming composition comprising nizatidine and a polymeric binder.

41. The method of claim 39, wherein said nizatidine-containing core is produced by granulating and milling and/or by extruding and spheronizing a polymer composition containing nizatidine.

42. A method of providing a subject with a timed, sustained release dosage of nizatidine which comprises orally administering to said subject a dosage form of claim 22.

43. The method according to claim 42, wherein said dosage form is administered two times a day.

44. The method according to claim 43, wherein said dosage form is administered once in the evening and once in the morning.

45. A method of treating a human having a gastro-intestinal disorder, comprising administering to the human a pulsatile release pharmaceutical dosage form comprising a therapeutically effective amount of a histamine H₂ antagonist.

46. The method of claim 45, wherein the histamine H₂ antagonist is selected from the group consisting of nizatidine, cimetidine, ranitidine and famotidine.

47. The method of claim 46, wherein the histamine H₂ antagonist is nizatidine.

48. The method of claim 45, wherein the pulsatile release pharmaceutical dosage form is bi-modal.

49. The method of claim 45, wherein the gastro-intestinal disorder is an acid peptic disease.

50. The method of claim 49, wherein the acid peptic disease is selected from the group consisting of Gastroesophogeal Reflux Disease (GERD), ulcers, heartburn, Nocturnal Acid Breakthrough, nighttime heartburn, regurgitation and retrosternal pain.

51. The method of claim 45, wherein about 100 mg to about 400 mg of a histamine H₂ antagonist is administered.

52. The method of claim 45, wherein the human is diabetic.

53. A method of treating a human having a gastro-intestinal disorder, comprising administering to the human once daily a bi-modal pulsatile release oral pharmaceutical dosage form comprising:
immediate release (IR) beads comprising a core particle containing nizatidine;
timed pulsatile release (TPR) beads, wherein said TPR beads comprise:
a core particle containing nizatidine; and
a pulse coating surrounding said core.

54. The method of claim 53, wherein about 150 mg to about 300 mg of nizatidine is administered.

55. A method of administering nizatidine, comprising administering orally to a human a bi-modal pulsatile release formulation comprising nizatidine that provides two peak blood plasma concentrations of nizatidine occurring from about 2.0 to about 4.0 hours apart, wherein the first peak concentration occurs within 2 hours after administration and wherein a therapeutic level of nizatidine is maintained for about 6 to about 8 hours after administration.

56. The method of claim 55, wherein the formulation is administered in the evening.

57. The method of claim 55, wherein the formulation is administered in the morning.

58. The method of claim 55, wherein about 150 mg to about 300 mg of nizatidine is administered.

59. The method of claim 55, wherein the formulation is administered once a day.

60. The method of claim 55, wherein the formulation is administered twice a day.

61. The method of claim 55, wherein about 150 mg of nizatidine is administered and the first peak concentration is from about 200 to about 800 ng/ml.

62. The method of claim 55, wherein about 150 mg of nizatidine is administered and the second peak concentration is from about 200 to about 800 ng/ml.

63. The method of claim 55, wherein about 300 mg of nizatidine is administered and the first peak concentration is from about 400 to about 1000 ng/ml.

64. The method of claim 55, wherein about 300 mg of nizatidine is administered and the second peak concentration is from about 600 to about 1200 ng/ml.

64. The method of claim 55, wherein the first peak concentration occurs within 1 hour after administration.

65. The method of claim 55, wherein the formulation comprises:
immediate release (IR) beads comprising a core particle containing nizatidine;
timed pulsatile release (TPR) beads, wherein said TPR beads comprise:
a core particle containing nizatidine; and
a pulse coating surrounding said core.

66. A method of administering nizatidine, comprising administering orally to a human a bi-modal pulsatile release formulation producing a first peak blood plasma concentration and a second peak blood level concentration, wherein the ratio of the first peak to the second peak is between about 75:25 and about 25:75.

67. The method of claim 66, wherein the ratio is between about 67:33 and about 33:67.

68. The method of claim 66, wherein the formulation comprises:
immediate release (IR) beads comprising a core particle containing nizatidine;
timed pulsatile release (TPR) beads, wherein said TPR beads comprise:
a core particle containing nizatidine; and
a pulse coating surrounding said core.

69. The method of claim 66, wherein the formulation is administered in the evening.

70. The method of claim 66, wherein the formulation is administered in the morning.

71. The method of claim 66, wherein about 150 mg to about 300 mg of nizatidine is administered.

72. The method of claim 66, wherein the formulation is administered once a day.

73. The method of claim 66, wherein the formulation is administered twice a day.

74. The method of claim 66, wherein about 150 mg of nizatidine is administered and the first peak concentration is from about 200 to about 800 ng/ml.

75. The method of claim 66, wherein about 150 mg of nizatidine is administered and the second peak concentration is from about 200 to about 800 ng/ml.

76. The method of claim 66, wherein about 300 mg of nizatidine is administered and the first peak concentration is from about 400 to about 1000 ng/ml.

77. The method of claim 66, wherein about 300 mg of nizatidine is administered and the second peak concentration is from about 600 to about 1200 ng/ml.

78. An extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 7.

79. An extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in FIG. 8.

80. An extended release (ER) oral dosage form comprising 150 mg of nizatidine, which after oral administration of a single one of said dosage forms twice daily in an adult human produces a blood plasma concentration of nizatidine ranging from 80 to 120% of the blood plasma concentration values per time period over the first twenty four hours shown in FIG. 9.

81. An extended release (ER) oral dosage form comprising 150 mg of nizatidine, which after oral administration of two of said dosage forms once daily in an adult human produces a blood plasma concentration of nizatidine ranging from 80 to 120% of the blood plasma concentration values per time period over the first eight hours shown in FIG. 9.

82. An extended release (ER) oral dosage form comprising 150 mg of nizatidine, which after oral administration of a single one of said dosage forms in an adult human produces a blood plasma concentration of nizatidine ranging from 80 to 120% of the blood plasma concentration values per time period over the first eight hours shown in any one of FIGS. 10-55.

83. An extended release (ER) oral dosage form comprising 150 mg of nizatidine, which after oral administration of a single one of said dosage forms in an adult human produces a blood plasma concentration of n-desmethylnizatidine ranging from 80 to 120% of the blood plasma concentration values per time period over the first eight hours shown in any one of FIGS. 56-101.
